(19) United States
(12) Patent Application Publication
(10) Pub. No.: US 2026/0168988 A1
(43) Pub. Date: Jun. 18, 2026

(54) GENETICALLY ENCODED REPORTER EXPRESSION SYSTEMS AND METHODS OF MAKING AND USING SAME

(71) Applicant: Duke University, Durham, NC (US)

(72) Inventor: Kathryn Meyer, Durham, NC (US)

(73) Assignee: Duke University, Durham, NC (US)

(21) Appl. No.: 18/709,703

(22) PCT Filed: Nov. 11, 2022
(86) PCT No.: PCT/US2022/079709

§ 371 Date: May 13, 2024

Related U.S. Application Data
(60) Provisional application No. 63/278,277, filed on Nov. 11, 2021.

(51) Int. Cl. G01N 33/50; C12N 9/06; C12N 9/78; C12N 15/90; C12Q 1/6897

(52) U.S. Cl. CPC G01N 33/5038; C12N 9/003; C12N 9/78; C12N 15/907; C12Q 1/6897; C12Y 105/01003; C12Y 305/04004; C12Y 305/04005

(57) ABSTRACT

Provided herein are compositions and methods for detection of N⁶-methyladenosine (m⁶A) methylation in live cells. The provided compositions include expression systems for detection of m⁶A methylation and identification of agents that modulate m⁶A methylation.

### PRIOR RELATED APPLICATION

This application claims the benefit of and priority to U.S. Provisional Application No. 63/278,277 filed on Nov. 11, 2021, which is hereby incorporated by reference in its entirety

### STATEMENT REGARDING FEDERALLY FUNDED RESEARCH

This invention was made with government support under Grant Nos. 1DP1DA046584-01 and 1R01MH118366-01 awarded by the National Institutes of Health/National Institute on Drug Abuse and National Institutes of Health/National Institute of Mental Health, respectively. The government has certain rights in the invention.

### FIELD

This disclosure describes compositions and methods for detecting RNA methylation in cells and tissues.

### REFERENCE TO A SEQUENCE LISTING SUBMITTED AS XML VIA EFS-WEB

The instant application contains a Sequence Listing which has been filed electronically in .xml format and is hereby incorporated by reference in its entirety. Said .xml copy, created on Nov. 10, 2022, is named 106707_1353583.xml and is 94 kilobytes in size.

### BACKGROUND

N⁶-methyladenosine (m⁶A) has emerged as an important regulator of cellular function. m⁶A is necessary for several physiological processes, such as stem cell maintenance, development, circadian rhythms, and learning and memory. In addition, abnormal levels of m⁶A in cells contributes to a variety of human diseases, including cardiovascular disease, viral infection, and several cancers. To date, strategies for detecting global changes in cellular m⁶A levels have focused on m⁶A antibodies, thin-layer chromatography (TLC), or UPLC-mass spectrometry (UPLC-MS). However, these methods are costly and suffer from major limitations. Further, these methods involve several sample processing steps, and require high amounts of input RNA. Moreover, antibody-based methods suffer from non-specificity, UPLC-MS requires specialized equipment, and TLC depends on radioactivity. More recently, alternatives to antibody-based global m⁶A mapping have been developed, but these methods also require substantial amounts of input RNA. Importantly, all current strategies involve isolation of RNA from cells and therefore do not enable real-time monitoring of m⁶A methylation in living cells. These limitations have been a major barrier for understanding how m⁶A is dynamically regulated in cells. In addition, no method exists for providing a direct readout of cellular mRNA methylation in a manner compatible with high-throughput screening (HTS). This has substantially limited drug discovery efforts and high-throughput studies designed to identify factors that regulate m⁶A in cells.

### SUMMARY

Provided herein is an expression system comprising: (a) a first DNA construct comprising a nucleic acid sequence encoding a fusion protein, wherein the fusion protein comprises an N⁶-methyladenosine (m⁶A) binding domain of a YT521-B homology (YTH) domain-containing protein fused to a catalytic domain of a cytidine deaminase or a catalytic domain of an adenosine deaminase; and (b) a second DNA construct comprising (i) a nucleic acid sequence encoding a heterologous polypeptide; (ii) a m⁶A sensor sequence; and (iii) a polypeptide encoding dihydrofolate reductase (DHFR).

In some embodiments, the m⁶A sensor sequence comprises SEQ ID NO: 16 (GACTTACGACAG). In some embodiments, the m⁶A binding domain is fused to the catalytic domain via a peptide linker. In some embodiments, the m⁶A binding domain comprises a polypeptide having at least 95% identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11. In some embodiments, the catalytic domain comprises a polypeptide having at least 95% identity to SEQ ID NO 12 or a catalytic fragment thereof, SEQ ID NO: 13 or a catalytic fragment thereof; SEQ ID NO: 14 or a catalytic fragment thereof; or SEQ ID NO: 15.

In some embodiments, a first vector comprises the first DNA construct. In some embodiments, a second vector comprises the second DNA construct. In some embodiments, a single vector comprises the first DNA construct and the second DNA construct. An exemplary construct comprising the first DNA construct and the second DNA construct is provided herein as SEQ ID NO: 45. This construct comprises a a nucleic acid encoding GFP, a m6A reporter sequence and DHFR; and a nucleic acid encoding APOBEC1-YTH (5′-3′).

In some embodiments, the nucleic acid sequence encoding a fusion protein and/or the nucleic acid sequence encoding a heterologous polypeptide and a polypeptide encoding dihydrofolate reductase (DHFR) are operably linked to a promoter. In some embodiments, the promoter is a constitutive or an inducible promoter. In some embodiments, the cytidine deaminase is apolipoprotein B mRNA editing enzyme catalytic subunit 1 (APOBEC-1).

In some embodiments, the heterologous polypeptide is a reporter protein. In some embodiments, the reporter protein is a fluorescent protein. In some embodiments, the fluorescent protein is a green fluorescent protein.

Also provided are host cells and populations of host cells comprising any of the expression systems described herein. Also provided is a non-human transgenic animal comprising any of the host cells described herein.

Further provided is a method for detecting m⁶A methylation-dependent expression of a heterologous polypeptide in one or more cells comprising: (a) introducing any of the expression systems described herein into one or more cells; and (b) detecting expression of the heterologous protein, wherein expression of the heterologous protein is indicative of m⁶A methylation-dependent expression of the heterologous polypeptide in the one or more cells.

Also provided is a method for detecting m⁶A methylation in one or more cells comprising: (a) introducing any of the reporter m⁶A expression systems described herein into one or more cells; and (b) detecting expression of the reporter protein.

Also provided is a method for identifying an agent that modulates m⁶A methylation in a cell comprising: (a) contacting one or more cells comprising any of the reporter protein expression systems described herein with an agent; and (b) detecting expression of the reporter protein, wherein a decrease in expression of the reporter protein as compared to expression of the reporter protein in the absence of the agent indicates the agent decreases m⁶A methylation, and wherein an increase in expression of the reporter protein as compared to expression of the reporter protein in the absence of the agent indicates the agent increases m⁶A methylation.

Also provided is a method for identifying an agent that inhibits METTL3-dependent methylation in a cell comprising: (a) contacting one or more cells comprising a m⁶A reporter expression system described herein with an agent; (b) detecting expression of the reporter protein, wherein a decrease in expression of the reporter protein as compared to expression of the reporter protein in the absence of the agent indicates the agent inhibits METTL3-dependent methylation.

Also provided is a method for identifying an agent that modulates m⁶A methylation in a non-human transgenic animal comprising: (a) contacting a non-human transgenic animal that comprises the expression system of any one of claims 15-17 with an agent; and (b) detecting expression of the reporter protein, wherein a decrease in expression of the reporter protein as compared to expression of the reporter protein in the absence of the agent indicates the agent decreases m⁶A methylation, and wherein an increase in expression of the reporter protein as compared to expression of the reporter protein in the absence of the agent indicates the agent increases m⁶A methylation.

Further provided is a method for identifying an agent that inhibits METTL3-dependent methylation in a non-human transgenic animal comprising: (a) contacting a non-human transgenic animal that comprises the expression system of any one of claims 15-17 with an agent; and (b) detecting expression of the reporter protein, wherein a decrease in expression of the reporter protein as compared to expression of the reporter protein in the absence of the agent indicates the agent inhibits METTL3-dependent methylation.

Kits comprising any of the nucleic acids or expression systems described herein are also provided. In some embodiments, the kit comprises (a) a nucleic acid sequence encoding any of the fusion proteins described herein and (b) a nucleic acid sequence comprising a nucleic acid sequence encoding a heterologous polypeptide, a m⁶A sensor sequence, and, a polypeptide encoding dihydrofolate reductase (DHFR). In some embodiments, the kit further comprises primers for amplification of one or more RNAs in a cell.

### DESCRIPTION OF THE FIGURES

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1A is a schematic of a m⁶A reporter according to certain embodiments of this disclosure. When methylation of the m⁶A sensor sequence (methylated: GACUUAUGACAG (SEQ ID NO: 37); unmethylated: GACUUACGACAG (SEQ ID NO: 38) that follows EGFP occurs, APO1-YTH (also referred to as APOBEC1-YTH) binds to m⁶A and edits nearby cytidine residues in the sensor sequence. This editing (C to U) generates a stop codon which blocks translation of the DHFR destabilization domain, thereby enabling EGFP fluorescence.

FIG. 1B shows HEK293T cells transfected with the m⁶A reporter alone (EGFP-DHFR) or together with APO1-YTH or APO1-YTH^{mut} according to certain embodiments of this disclosure. Strong EGFP fluorescence is detected only with APO1-YTH co-transfection.

FIG. 1C is a Western blot analysis of cell lysates 24 h after co-transfection with the indicated plasmids as in FIG. 1B according to certain embodiments of this disclosure. EGFP is only observed when the m⁶A reporter is co-transfected with APO1-YTH.

FIG. 1D shows C to T conversion with a CGA sequence according to certain embodiments of this disclosure. RNA was collected from cells transfected in FIG. 1B and RT-PCR was performed to amplify the m⁶Asensor region of the m⁶A reporter mRNA. Sanger sequencing traces show C to T conversion (arrow) within the CGA sequence (generating a UGA stop codon in the mRNA only in cells co-transfected with the m⁶A reporter and APO1-YTH). In FIG. 1D, the C to T conversion occurs in a m⁶A sensor sequence (GGACTTACGACAGTT) (SEQ ID NO: 39).

FIG. 2A provides data showing the characterization of the residues that contribute to m⁶A reporter activity according to certain embodiments of this disclosure. HEK293T cells were co-transfected with APO1-YTH and the indicated reporters, or with the C to U mutant reporter alone. 24 h later, images were acquired to assess EGFP fluorescence. The sensor sequence of each m⁶A reporter used (GACUUACGACAG (SEQ ID NO: 40); GACUUAUGACAG (SEQ ID NO: 41); GGCUUACGACAG (SEQ ID NO: 42); and GACUUACGAGAG (SEQ ID NO: 43) is shown above each image set. The two m⁶A sites in the reporter are shown and the stop codon that results from C to U editing of the methylated reporter is underlined. Asterisks indicate mutated bases in the indicated reporter variant.

FIG. 2B shows Western blotting of cell lysates collected from HEK293T cells transfected as in FIG. 2A confirming EGFP protein production and no EGFP-DHFR production in the C to U reporter, according to certain embodiments of this disclosure. FIG. 2B also shows reduced EGFP protein production in the two m⁶A mutant reporters.

FIG. 2C shows RT-PCR performed on cells transfected as in FIG. 2A according to certain embodiments of this disclosure. Sanger sequencing was performed on the reporter RNA. C to U editing (C to T in cDNA) was diminished in the two m⁶A mutant reporters compared to the original (WT) m⁶A reporter. The m⁶A sensor sequence (SEQ ID NO: 39) is shown.

FIG. 3A shows the inducible expression of the m⁶A reporter in stable cell lines according to certain embodiments of this disclosure. HEK293T stable cells were generated which express both the m⁶A reporter and APO1-YTH. The m⁶A reporter was constitutively expressed from the CMV reporter, while APO1-YTH was inducibly expressed after treatment of cells with doxycycline (dox). Shown are m⁶A reporter stable cells after dox induction of APO1-YTH.

FIG. 3B is a Western blot confirming the induction of APO1-YTH as well as EGFP expression in dox-treated cells according to certain embodiments of this disclosure. Cyclophilin A (CycloA) is shown as a loading control.

FIG. 4A shows that a m⁶A sensor responds to reduced methyltransferase 3, N6-adenosine-methyltransferase complex catalytic subunit (METTL3) levels according to certain embodiments of this disclosure. METTL3 degron cells expressing the m⁶A sensor system were treated with auxin to degrade METTL3, which reduced m⁶A sensor fluorescence.

FIG. 4B is a Western blot of cells in FIG. 4A showing reduction of GFP expression even with modest METTL3 loss according to certain embodiments of this disclosure.

FIG. 4C shows RT-PCR/Sanger sequencing confirming reduced C-to-U editing of the m⁶A sensor sequence after METTL3 depletion (+Auxin) according to certain embodiments of this disclosure.

FIG. 5A shows that the m⁶A sensor responds to METTL3 overexpression (OE) according to certain embodiments of this disclosure. HEK293T cells transfected with the m⁶A sensor system exhibit increased GFP fluorescence when METTL3 is overexpressed (METTL3 OE).

FIG. 5B is a Western blot of cells in FIG. 5A showing increased GFP expression, even with modest METTL3 OE according to certain embodiments of this disclosure.

FIG. 5C shows RT-PCR/Sanger sequencing of the m⁶A sensor sequence confirming increased % C2U after METTL3 OE according to certain embodiments of this disclosure.

FIG. 6A shows that METTL3 inhibition reduces the m⁶A sensor signal, according to certain embodiments of this disclosure. Treatment of HEK293T cells expressing the m⁶A sensor system with STM2457 reduces GFP fluorescence.

FIG. 6B shows that RNA from cells in FIG. 6A was subjected to RT-PCR/Sanger sequencing. STM2457 treatment reduced C-to-U editing of the m⁶A sensor sequence according to certain embodiments of this disclosure.

FIG. 6C is a Western blot of cells in FIG. 6A showing reduction of GFP expression after STM2457 treatment according to certain embodiments of this disclosure.

FIG. 7A shows that a m⁶A sensor is not a target of NMD or m⁶A-mediated degradation according to certain embodiments of this disclosure. METTL3 degron cells were treated with auxin for 48 hr and then transfected with the m⁶A sensor system. RT-qPCR of the m⁶A reporter mRNA was performed 24 hr later.

FIG. 7B shows that a m⁶A sensor is not a target of NMD or m⁶A-mediated degradation according to certain embodiments of this disclosure. HEK293T cells expressing the m6A sensor system were treated with CHX for 24 h to inhibit NMD, followed by RT-qPCR of the m⁶A reporter mRNA.

FIG. 8A shows that a m⁶A sensor detects cellular m⁶A according to certain embodiments of this disclosure. Cells expressing the m⁶A reporter mRNA show GFP fluorescence only in the presence of APO1-YTH.

FIG. 8B is a Western blot confirming that GFP is expressed only in cells co-expressing the m⁶A reporter mRNA and APO1-YTH according to certain embodiments of this disclosure. HA tag blot indicates expression of APO1-YTH and APO1-YTHmut. Cyclophilin A=loading control.

FIG. 8C shows RT-PCR/Sanger sequencing of the m⁶A sensor sequence according to certain embodiments of this disclosure. C-to-U editing of the convertible cytidines (arrows) is observed only in cells expressing APO1-YTH.

FIG. 8D shows SELECT verification of the presence of m⁶A in a consensus sequence adenosine (GAC) of the sensor sequence according to certain embodiments of this disclosure. Nearby non-consensus adenosines were unmethylated. SELECT targeting the 3′UTR of endogenous ACTB shows that the m⁶A sensor sequence is methylated at similar levels as endogenous ACTB. Dotted line represents the minimum cutoff value of SELECT that indicates the presence of m⁶A.

FIG. 9A shows that GFP-DHFR does not contribute to background fluorescence according to certain embodiments of this disclosure. HEK293T cells expressing the m⁶A sensor mRNA were treated with trimethoprim (TMP) to stabilize GFP-DHFR. TMP treatment leads to GFP fluorescence, but untreated cells are dark.

FIG. 9B is a Western blot showing increased GFP-DHFR protein after TMP treatment according to certain embodiments of this disclosure. As expected, no GFP product is produced in the absence of APO1-YTH.

FIG. 9C shows that no C-to-U editing of the sensor sequence is observed in the absence of APO1-YTH according to certain embodiments of this disclosure.

FIG. 10A shows expression of GFP and GFP-PEST. GFP-PEST or GFP versions of the m⁶A sensor system were transfected into HEK293T cells, followed by treatment with CHX to inhibit protein synthesis according to certain embodiments of this disclosure. GFP-PEST has a shorter half-life than GFP.

FIG. 10B shows RT-PCR/Sanger sequencing confirming equal editing of the m⁶A sensor sequence when using GFP or GFP-PEST versions of the m⁶A reporter mRNA according to certain embodiments of this disclosure.

FIG. 11 shows expression of a m⁶A sensor system with dsRed as an internal control in HEK293 cells, according to certain embodiments of this disclosure.

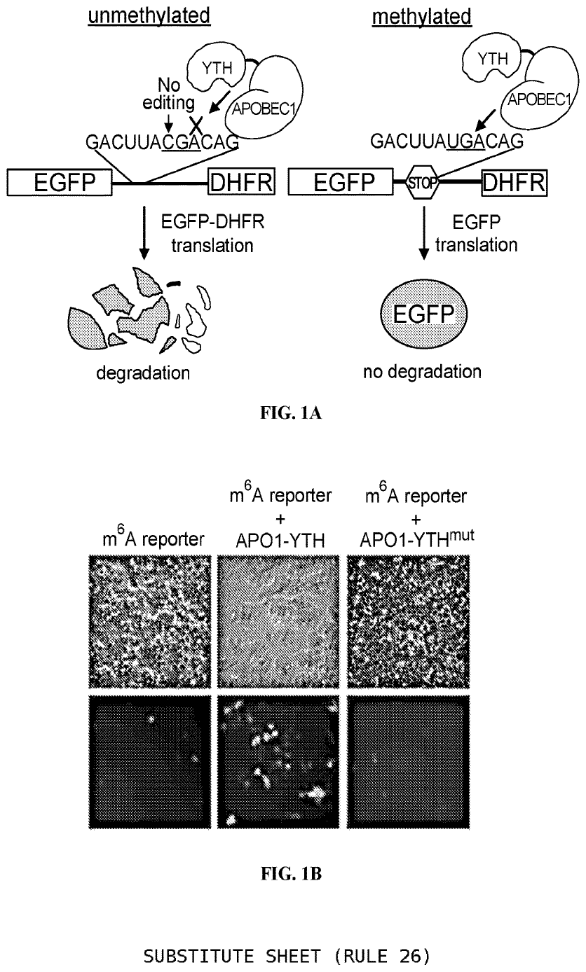

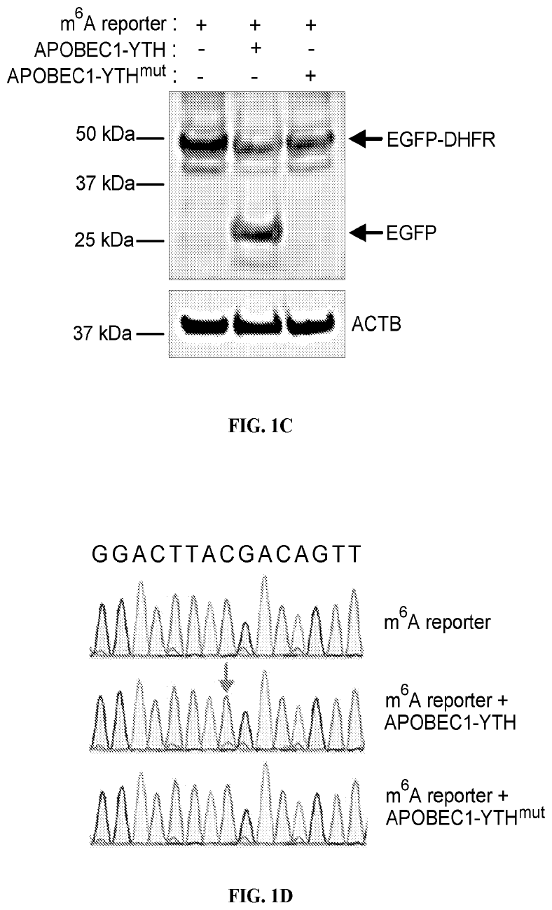

FIG. 1B shows that a modified global KO screen resulted in increased METTL3 indels in dsRed+/GFP− cells, according to certain embodiments of this disclosure.

FIG. 11C shows that the m⁶A sensor sequence from dsRed+/GFP− cells shows no editing compared to dsRed+/GFP+ cells, as confirmed by Sanger sequencing.

FIG. 12 shows stable expression of APO1-YTH in cells. A Western blot shows dox-inducible APO1-YTH expression across a variety of stable cell lines according to certain embodiments of this disclosure.

### DETAILED DESCRIPTION

The following description recites various aspects and embodiments of the present compositions and methods. No particular embodiment is intended to define the scope of the compositions and methods. Rather, the embodiments merely provide non-limiting examples of various compositions and methods that are at least included within the scope of the disclosed compositions and methods. The description is to be read from the perspective of one of ordinary skill in the art; therefore, information well known to the skilled artisan is not necessarily included.

Unless defined otherwise, all technical and scientific terms used herein have the same meaning as commonly understood by one of ordinary skill in the art to which this disclosure belongs. All patents, patent applications and publications referred to throughout the disclosure herein are incorporated by reference in their entirety.

Articles “a” and “an” are used herein to refer to one or to more than one (i.e. at least one) of the grammatical object of the article. By way of example, “an element” means at least one element and can include more than one element.

The use of any and all examples or exemplary language (e.g., “such as”) provided herein, is intended merely to better illustrate the invention and does not pose a limitation on the scope of the invention unless otherwise claimed.

The terms “may,” “may be,” “can,” and “can be,” and related terms are intended to convey that the subject matter involved is optional (that is, the subject matter is present in some examples and is not present in other examples), not a reference to a capability of the subject matter or to a probability, unless the context clearly indicates otherwise.

“About” is used to provide flexibility to a numerical range endpoint by providing that a given value may be “slightly above” or “slightly below” the endpoint without affecting the desired result.

The terms “optional” and “optionally” mean that the subsequently described event, circumstance, or material may or may not occur or be present, and that the description includes instances where the event, circumstance, or material occurs or is present as well as instances where it does not occur or is not present.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

### I. Expression Systems

Provided herein is an expression system comprising: (a) a first DNA construct comprising a nucleic acid sequence encoding a fusion protein, wherein the fusion protein comprises an N⁶-methyladenosine (m⁶A) binding domain of a YT521-B homology (YTH) domain-containing protein fused to a catalytic domain of a cytidine deaminase or a catalytic domain of an adenosine deaminase (e.g., APOBEC1); and (b) a second DNA construct comprising (i) a nucleic acid sequence encoding a heterologous polypeptide; (ii) a m⁶A sensor sequence; and (iii) a polypeptide encoding dihydrofolate reductase (DHFR). The nucleic acid sequence encoding a heterologous polypeptide; (ii) a m⁶A sensor sequence; and (iii) a polypeptide encoding dihydrofolate reductase (DHFR) is also referred to as the mRNA reporter sequence. Also provided is a nucleic acid sequence comprising a nucleic acid sequence encoding a heterologous polypeptide, a m⁶A sensor sequence, and, a polypeptide encoding dihydrofolate reductase (DHFR).

Any of the nucleic acid sequences provided herein can be included in expression cassettes for expression in a host cell or an organism of interest. The cassette will include 5′ and 3′ regulatory sequences operably linked to a recombinant nucleic acid provided herein that allows for expression of the modified polypeptide. A nucleic acid is “operably linked” when it is placed into a functional relationship with another nucleic acid sequence. Numerous promoters can be used in the constructs described herein. A promoter is a region or a sequence located upstream and/or downstream from the start of transcription that is involved in recognition and binding of RNA polymerase and other proteins to initiate transcription. The promoter can be a eukaryotic or a prokaryotic promoter. In some embodiments the promoter is an inducible promoter. In some embodiments, the promoter is a constitutive promoter.

In some embodiments, the nucleic acid sequence encoding a fusion protein comprising an N⁶-methyladenosine (m⁶A) binding domain of a YT521-B homology (YTH) domain-containing protein fused to a catalytic domain of a cytidine deaminase or a catalytic domain of an adenosine deaminase is operably linked to an inducible promoter, e.g., a tetracycline inducible promoter; and the nucleic acid construct encoding the mRNA reporter sequence is operably linked to a constitutive promoter (e.g., a CMV promoter).

A “constitutive” promoter is a promoter that is active under most environmental and developmental conditions. Examples of constitutive promoters include, but are not limited to, a CMV promoter, a U6 promoter, a PGK promoter, a EF-1α promoter and a SV40 promoter.

An “inducible” promoter is a promoter that is active under environmental or developmental regulation, for example, regulated by the presence or absence of a drug. Examples of inducible promoters include, but are not limited to, the pL promoter (induced by an increase in temperature), the pBAD promoter, (induced by the addition of arabinose to the growth medium). the tetracycline-controlled transcriptional activation system (Tet-On/Tet-Off, Bujard and Gossen, PNAS, 89(12):5547-5551 (1992)), the Lac switch inducible system (Wyborski et al., Environ Mol Mutagen, 28(4):447-58 (1996)), the ecdysone-inducible gene expression system (No et al., PNAS, 93(8):3346-3351 (1996)), the cumate gene-switch system (Mullick et al., BMC Biotechnology, 6:43 (2006)), and the tamoxifen-inducible gene expression (Zhang et al., Nucleic Acids Research, 24:543-548 (1996)). Furthermore, a Cre-loxP inducible system can also be used, as well as a Flp recombinase inducible promoter system, both of which are known in the art.

In some embodiments, the promoter is a cell-specific or tissue-specific promoter. When using a cell- or tissue-specific promoter, expression occurs primarily, but not exclusively, in a particular cell or tissue. For example, expression can occur in at least 90%, 95%, or 99% of the targeted cell or tissue. It will be understood, however, that tissue-specific promoters may have a detectable amount of background or base activity in those tissues where they are mostly silent.

Examples of tissue-specific promoters include, but are not limited to, liver-specific promoters (e.g., APOA2, SERPINA1, CYP3A4, MIR122), pancreatic-specific promoters (e.g., insulin, insulin receptor substrate 2, pancreatic and duodenal homeobox 1, Aristaless-like homeobox 3, and pancreatic polypeptide), cardiac-specific promoters (e.g., myosin, heavy chain 6, myosin, light chain 2, troponin I type 3, natriuretic peptide precursor A, solute carrier family 8), central nervous system promoters (e.g., glial fibrillary acidic protein, intemexin neuronal intermediate filament protein, Nestin, myelin-associated oligodendrocyte basic protein, myelin basic protein, tyrosin hydroxylase, and Forkhead box A2), skin-specific promoters (e.g., Filaggrin, Keratin 14 and transglutaminase 3), pluripotent and embryonic germ layer promoters (e.g., POU class 5 homeobox 1, Nanog homeobox, Nestin, and MicroRNA 122).

The cassette may additionally contain at least one additional gene or genetic element to be cotransformed into the organism. Where additional genes or elements are included, the components are operably linked. Alternatively, the additional gene(s) or element(s) can be provided on multiple expression cassettes. Such an expression cassette is provided with a plurality of restriction sites and/or recombination sites for insertion of the polynucleotides to be under the transcriptional regulation of the regulatory regions. The expression cassette may additionally contain a selectable marker gene. The expression cassette will include in the 5′ to 3′ direction of transcription: a transcriptional and translational initiation region (i.e., a promoter), a polynucleotide of the invention, and a transcriptional and translational termination region (i.e., termination region) functional in the cell or organism of interest. The promoters of the invention are capable of directing or driving expression of a coding sequence in a host cell. The regulatory regions (i.e., promoters, transcriptional regulatory regions, and translational termination regions) may be endogenous or heterologous to the host cell or to each other. As used herein the term “heterologous” refers to a nucleotide sequence or polypeptide not normally found in a given cell in nature. As such, a heterologous nucleotide sequence or heterologous polypeptide may be: (a) foreign to its host cell (i.e., is exogenous to the cell); (b) naturally found in the host cell (i.e., endogenous) but present at an unnatural quantity in the cell (i.e., greater or lesser quantity than naturally found in the host cell); or (c) be naturally found in the host cell but positioned outside of its natural locus.

Additional regulatory signals include, but are not limited to, transcriptional initiation start sites, operators, activators, enhancers, other regulatory elements, ribosomal binding sites, an initiation codon, termination signals, and the like. See Sambrook et al. (1992) Molecular Cloning: A Laboratory Manual, ed. Maniatis et al. (Cold Spring Harbor Laboratory Press, Cold Spring Harbor, N.Y.) (hereinafter “Sambrook 11”); Davis et al., eds. (1980) Advanced Bacterial Genetics (Cold Spring Harbor Laboratory Press), Cold Spring Harbor, N.Y., and the references cited therein.

In preparing the expression cassette, the various DNA fragments may be manipulated, to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Further provided is a vector comprising a nucleic acid or expression cassette set forth herein. The vector is contemplated to have the necessary functional elements that direct and regulate transcription of the inserted nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers which can serve to select for cells containing the vector or the vector containing the insert, RNA splice junctions, a transcription termination region, or any other region which may serve to facilitate the expression of the inserted gene or hybrid gene (See generally, Sambrook et al. Molecular Cloning: A Laboratory Manual, 4^{th} ed., Cold Spring Harbor Laboratory Press, Cold Spring Harbor, 2012). The vector, for example, can be a plasmid.

In some embodiments, a vector comprises the first DNA construct. In some embodiments, a vector comprises the second DNA construct. In some embodiments, a vector comprises the first and second DNA construct. In some embodiments, the vector is a plasmid. In some embodiments, a vector comprises the first DNA construct, the second DNA construct and a nucleic acid encoding a selectable marker. In some embodiments, the first DNA construct and the second DNA construct are operably linked to a first promoter, and the nucleic acid sequence encoding a selectable marker is operably linked to a second promoter (i.e., a promoter that is different from the first promoter). In some embodiments, the selectable marker is a fluorescent protein, that is different from the fluorescent protein encoded by second DNA construct, for example, dsRed. An exemplary dual-promoter construct comprising: (1) a nucleic acid sequence encoding GFP, a m⁶A reporter sequence and DHFR; (2) a nucleic acid sequence encoding a fusion protein (APOBEC1-YTH); and (3) a nucleic acid sequence encoding dsRed is provided herein as SEQ ID NO: 46).

There are numerous E. coli expression vectors known to one of ordinary skill in the art, which are useful for the expression of any of the nucleic acid sequences described herein (e.g., any of the fusion proteins described herein). Other microbial hosts suitable for use include bacilli, such as Bacillus subtilis, and other enterobacteriaceae, such as Salmonella, Senatia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. Additionally, yeast expression can be used.

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

As used throughout, a “fusion protein” is a protein comprising two different polypeptide sequences, i.e. a binding domain and a catalytic domain, that are joined or linked to form a single polypeptide. The two amino acid sequences are encoded by separate nucleic acid sequences that have been joined so that they are transcribed and translated to produce a single polypeptide. In some embodiments, the fusion protein comprises, in the following order, a m⁶A binding domain, and a catalytic domain of a cytidine deaminase or an adenosine deaminase.

As used throughout, “m⁶A” refers to posttranscriptional methylation of an adenosine residue in the RNA of prokaryotes and eukaryotes (e.g., mammals, insects, plants and yeast).

As used throughout an “m⁶A sensor sequence” is a sequence comprising one or more m⁶A methylation consensus motifs (GAC). The m⁶A sensor sequence can also comprise at least one sequence that can be converted to a stop codon when the m⁶A sensor sequence is methylated in the cell. In the constructs described herein, the m⁶A sensor sequence is in-frame with the nucleic acid encoding the heterologous protein, e.g. a reporter protein. The m⁶A sensor sequence is flanked by the nucleic acid sequence encoding the heterologous protein (e.g., reporter protein) and the nucleic acid sequence encoding a destabilization domain, e.g., DHFR. When the construct is methylated in the cell, a C to U modification generates a stop codon in the m⁶A sensor sequence. The stop codon prevents expression of the destabilization domain, thus preventing degradation of the heterologous protein. Exemplary m6A sensor sequences include, but are not limited to, a nucleic acid sequence comprising, consisting of, or consisting essentially of, SEQ ID NO: 16, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 and SEQ ID NO: 58. Nucleic acid sequences having at least 90% identity with a nucleic acid sequence comprising, consisting essentially of, or consisting of SEQ ID NO: 16, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 and SEQ ID NO: 58 are also provided. One of skill in the art would understand that these sequences are merely exemplary because any m⁶A sensor sequence comprising at least one m⁶A methylation consensus motif (GAC) (e.g., one, two, three, four etc.) can be used as a sensor sequence.

As used throughout, a m⁶A binding domain of a YT521-B homology (YTH) domain-containing protein is a polypeptide fragment of a YTH domain-containing protein that binds to m⁶A-containing sequence (e.g., a RNA, such as a mRNA or a m⁶A sensor sequence). The m⁶A binding domain derived from a YT521-B homology (YTH) domain-containing protein can be of any size as long as it retains binding activity and is not the full-length YTH domain-containing protein. In some embodiments, the binding domain retains at least about 75%, 80%, 90%, 95%, or 99% of the binding activity of the wildtype YTH domain-containing protein from which the binding domain is derived.

In some embodiments, the DNA construct encodes a m⁶A binding domain comprising a polypeptide having at least 95% identity, for example, at least about 95%, 96%, 97%, 98% or 99% identity, to SEQ ID NO: 1 (amino acid sequence of YTHDF2-YTH, a m⁶A binding domain of YTHDF2), SEQ ID NO: 2 (amino acid sequence of YTHDF2-YTH_W432A_W486A, a mutated m⁶A binding domain of YTHDF2), SEQ ID NO: 3 (amino acid sequence of YTHDF2-YTHmut, an amino acid sequence that includes the YTH domain of YTHDF2, and does not include the m6A-binding domain), SEQ ID NO: 4 (amino acid sequence of YTHDF2-YTHmut, an amino acid sequence comprising SEQ ID NO: 3, with a W432A mutation and a W486a mutation), SEQ ID NO: 5 (amino acid sequence of YTHDF2-YTH D422N, a mutated m⁶A binding domain of YTHDF2), SEQ ID NO: 6 (amino acid sequence of a m⁶A binding domain of YTHDF1), SEQ ID NO: 7 (amino acid sequence of YTHDF1mut, an amino acid sequence that includes the YTH domain of YTHDF2, and does not include the m⁶A-binding domain), SEQ ID NO: 8 (amino acid sequence of YTHDF1 D401N, a mutated m⁶A binding domain of YTHDF1), SEQ ID NO: 9 (amino acid sequence of a m⁶A binding domain of YTHDF3); SEQ ID NO: 10 (amino acid sequence of a m⁶A binding domain of YTHDC1) or SEQ ID NO: 11 (amino acid sequence of a m⁶A binding domain of YTHDC2).

As used throughout, a catalytic domain of a cytidine deaminase is a polypeptide comprising a cytidine deaminase, for example, Apolipoprotein B MRNA Editing Enzyme Catalytic Subunit (APOBEC1 or APO1), activation induced cytidine deaminase (AICDA) or Apolipoprotein B mRNA editing enzyme, catalytic polypeptide-like 3A (APOBEC3A), or a catalytic fragment thereof, that catalyzes deamination of cytidine (“C”) to uridine (“U”) in RNA molecules. As used throughout, a catalytic domain of an adenosine deaminase, is a polypeptide comprising an adenosine deaminase, for example, double-stranded RNA-specific adenosine deaminase (ADAR1), or a catalytic fragment thereof, that catalyzes deamination of adenosine (“A”) to inosine (“I”) in RNA molecules. In some embodiments, the catalytic domain retains at least about 75%, 80%, 90%, 95%, or 99% of the enzymatic activity of the wildtype deaminase from which the domain is derived.

In some embodiments, the catalytic domain comprises a polypeptide having at least 95% identity, for example, at least about 95%, 96%, 97%, 98% or 99% identity, to SEQ ID NO: 12 (amino acid sequence of rAPOBEC1) or its catalytic domain (SEQ ID NO: 60), SEQ ID NO: 13 (amino acid sequence of hAICDA) or its catalytic domain (SEQ ID NO: 61); SEQ ID NO: 14 (amino acid sequence of hAPOBEC3A) or its catalytic domain (SEQ ID NO: 62); or SEQ ID NO: 15 (amino acid sequence of ADAR1) or its catalytic domain (SEQ ID NO: 63).

The catalytic domain can also comprise a polypeptide having at least 95% identity to SEQ ID NO: 17 (amino acid sequence of catalytic domain of ADAR2), as set forth in U.S. Patent Application Publication No. 20190010478.

In some embodiments, the DNA construct encodes a m⁶A binding domain fused to the catalytic domain via a peptide linker. The peptide linker can be about 2 to about 150 amino acids in length. For example, the linker can be a linker of from about 5 to about 20 amino acids in length, from about 5 to about 25 amino acids in length, from about 10 to about 30 amino acids in length, 5 to about 35 amino acids in length, from about 5 to about 40 amino acids in length, from about 5 to about 45 amino acids in length, from about 5 to about 50 amino acids in length, from about 5 to about 55 amino acids in length, from about 5 to about 60 amino acids in length, from about 5 to about 65 amino acids in length, from about 5 to about 70 amino acids in length, from about 5 to about 75 amino acids in length, from about 5 to about 80 amino acids in length, from about 5 to about 85 amino acids in length, from about 5 to about 90 amino acids in length, from about 5 to about 95 amino acids in length, from about 5 to about 100 amino acids in length, from about 5 to about 105 amino acids in length, from about 5 to about 110 amino acids in length, from about 5 to about 115 amino acids in length, from about 5 to about 120 amino acids in length, from about 5 to about 125 amino acids in length, from about 5 to about 130 amino acids in length, from about 5 to about 135 amino acids in length, from about 5 to about 140 amino acids in length, from about 5 to about 145 amino acids in length, or from about 5 to about 150 amino acids in length.

Exemplary peptide linkers include, but are not limited to, peptide linkers comprising SEQ ID NO: 18 (SGSETPGTSESATPE), SEQ ID NO: 19 (SGSETPGTSESATPES), SEQ ID NO: 20 ((GGGGS)₃), SEQ ID NO: 21 ((GGGGS)₁₀), SEQ ID NO: 59 ((GGGGS)₂₀), SEQ ID NO: 22 (A(EAAAK)₃A), SEQ ID NO: 23 (A(EAAAK)₁₀A), or SEQ ID NO: 24 (A(EAAAK)₂₀A).

In some embodiments, the fusion protein further comprises a localization element. In some embodiments, the localization element is fused to the N-terminus or the C-terminus of the fusion protein. As used herein, a localization element targets or localizes the fusion protein to one or more subcellular compartments. Subcellular compartments include but are not limited to, the nucleus, the endoplasmic reticulum, the mitochondria, chromatin, the cellular membrane, and RNA granules (for example, P-bodies, stress granules and transport granules). In some embodiments, the fusion protein can be targeted to the nuclear lamina, nuclear speckles nuclear paraspeckles in the nucleus of a cell. In some embodiments, the protein can be targeted to the outer mitochondrial membrane or the inner mitochondrial membrane.

Exemplary localization elements include, but are not limited to, a peptide comprising a nuclear localization signal, for example, SEQ ID NO: 27 (PKKKRKV), a peptide comprising a nuclear export signal, for example, SEQ ID NO: 28 (LPPLERLTL), a peptide comprising an endoplasmic reticulum targeting sequence, for example, SEQ ID NO: 29 (MDPVVVLGLCLSCLLLLSLWKQSYGGG), or SEQ ID NO: 30 (METDTLLLWVLLLWVPGSTGD), a peptide comprising a Myc tag, for example, SEQ ID NO: 31 (EQKLISEEDL), a peptide comprising a V5 tag, for example, SEQ ID NO:32 (GKPIPNPLLGLDST) or SEQ ID NO: 33 (IPNPLLGLD), a peptide comprising a FLAG tag, for example, SEQ ID NO: 34 (DYKDDDDK), a peptide comprising a 3×FLAG tag, for example, SEQ ID NO: 35 (DYKDHDGDYKDHDIDYKDDDDK) and a peptide comprising a DHFR destabilization domain, for example, SEQ ID NO: 36 (ISLIAALAVDHVIGMETVMPWNLPADLAWFKRNTLNKPVI MGRHTWESIGRPLPGRKNIILSSQPSTDDRVTWVKSVDEAIAACGDVPEIMVIGGGR VYEQFLPKAQKLYLTHIDAEVEGDTHFPDYEPDDWESVFSEFHDADAQNSHSYCFEI LERR).

Modifications to any of the polypeptides or proteins provided herein are made by known methods. By way of example, modifications are made by site specific mutagenesis of nucleotides in a nucleic acid encoding the polypeptide, thereby producing a DNA encoding the modification, and thereafter expressing the DNA in recombinant cell culture to produce the encoded polypeptide. Techniques for making substitution mutations at predetermined sites in DNA having a known sequence are well known. For example, M13 primer mutagenesis and PCR-based mutagenesis methods can be used to make one or more substitution mutations. Any of the nucleic acid sequences provided herein can be codon-optimized to alter, for example, maximize expression, in a host cell or organism. SEQ ID NOs: 25 and 26 are exemplary codon-optimized nucleic acids for expression and purification of APOBEC1-YTH and APOBEC1-YTHmut, respectively.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23(4): 581-587 (2013); Xie et la. “Adding amino acids to the genetic repertoire,” 9(6): 548-54 (2005)); and all references cited therein. B and y amino acids are known in the art and are also contemplated herein as unnatural amino acids.

As used herein, a chemically modified amino acid refers to an amino acid whose side chain has been chemically modified. For example, a side chain can be modified to comprise a signaling moiety, such as a fluorophore or a radiolabel. A side chain can also be modified to comprise a new functional group, such as a thiol, carboxylic acid, or amino group. Post-translationally modified amino acids are also included in the definition of chemically modified amino acids.

Also contemplated are conservative amino acid substitutions. By way of example, conservative amino acid substitutions can be made in one or more of the amino acid residues, for example, in one or more lysine residues of any of the polypeptides provided herein. One of skill in the art would know that a conservative substitution is the replacement of one amino acid residue with another that is biologically and/or chemically similar. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

By way of example, when an arginine to serine is mentioned, also contemplated is a conservative substitution for the serine (e.g., threonine). Nonconservative substitutions, for example, substituting a lysine with an asparagine, are also contemplated.

Any of the polypeptides described herein can further comprise a detectable moiety, for example, a fluorescent protein or fragment thereof. Examples of fluorescent proteins include, but are not limited to, yellow fluorescent protein (YFP, for example, Venus), green fluorescent protein (GFP), and red fluorescent protein (RFP) as well as derivatives, for example, mutant derivatives, of these proteins. See, for example, Chudakov et al. “Fluorescent Proteins and Their Applications in Imaging Living Cells and Tissues,” Physiological Reviews 90(3): 1103-1163 (2010); and Specht et al., “A Critical and Comparative Review of Fluorescent Tools for Live-Cell Imaging,” Annual Review of Physiology 79: 93-117 (2017))

Any of the polypeptides described herein can further comprise an affinity tag, for example a polyhistidine tag ((His)₆) (SEQ ID NO: 44), albumin-binding protein, alkaline phosphatase, an AU1 epitope, an AU5 epitope, a biotin-carboxy carrier protein (BCCP) or a FLAG epitope, to name a few. See, Kimple et al. “Overview of Affinity Tags for Protein Purification, Curr. Protoc. Protein Sci. 73: Unit-9.9 (2013).

Recombinant nucleic acids encoding any of the polypeptides described herein are also provided. For example, a recombinant nucleic acid encoding a polypeptide that has at least 95%, for example, at least about 95%, 96%, 97%, 98% or 99%, identity to any one of SEQ ID NOs 1-15, 17, 25 and 32 are also provided.

As used throughout, the term “nucleic acid” or “nucleotide” refers to deoxyribonucleic acids (DNA) or ribonucleic acids (RNA) and polymers thereof in either single- or double-stranded form. It is understood that when an RNA is described, its corresponding cDNA is also described, wherein uridine is represented as thymidine. Unless specifically limited, the term encompasses nucleic acids containing known analogues of natural nucleotides that have similar properties as the reference nucleic acid and are metabolized in a manner similar to naturally occurring nucleotides. A nucleic acid sequence can comprise combinations of deoxyribonucleic acids and ribonucleic acids. Such deoxyribonucleic acids and ribonucleic acids include both naturally occurring molecules and synthetic analogues. The polynucleotides of the invention also encompass all forms of sequences including, but not limited to, single-stranded forms, double-stranded forms, hairpins, stem-and-loop structures, and the like.

As used throughout, RNA can be messenger RNA (mRNA), transfer RNA (tRNA), small nuclear RNA (snRNA), a regulatory RNA, a transfer-messenger RNA (tmRNA), ribosomal RNA (rRNA), microRNA (miRNA), long noncoding RNA (lncRNA) or circular RNA (circRNA).

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977)Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see. e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

### II. Reporter Expression Systems

Provided herein is an expression system comprising: (a) a first DNA construct comprising a nucleic acid sequence encoding a fusion protein, wherein the fusion protein comprises an N⁶-methyladenosine (m⁶A) binding domain of a YT521-B homology (YTH) domain-containing protein fused to a catalytic domain of a cytidine deaminase or a catalytic domain of an adenosine deaminase; and (b) a second DNA construct comprising (i) a nucleic acid sequence encoding a reporter protein; (ii) a m⁶A sensor sequence; and (iii) a polypeptide encoding dihydrofolate reductase (DHFR). In some embodiments, the DNA second construct of any reporter expression system described herein encodes, in the following order, a reporter mRNA comprising a reporter protein, a m⁶A sensor sequence and a polypeptide encoding DHFR.

The expression systems described herein can be used to detect or monitor m⁶A methylation in in vitro, ex vivo or in vivo cells.

As used herein a reporter protein or polypeptide refers to a protein that can be used as an indicator of the occurrence or level of a particular biological process, activity, event, or state in a cell or organism. Reporter proteins typically have one or more properties or enzymatic activities that allow them to be readily measured or that allow selection of a cell that expresses the reporter protein. In general, a cell can be assayed for the presence of a reporter protein by measuring the reporter protein itself or an enzymatic activity of the reporter protein. Detectable characteristics or activities that a reporter protein may have include but are not limited to, fluorescence, bioluminescence, ability to catalyze a reaction that produces a fluorescent or colored substance in the presence of a suitable substrate, or other readouts based on emission and/or absorption of photons (light). Typically, a reporter protein is a protein that is not endogenously expressed by a cell or organism in which the reporter protein is used. In some embodiments, a nucleic acid encoding a reporter protein is codon-optimized for expression in mammalian cells.

In some embodiments, the reporter protein is a fluorescent protein. Examples of suitable fluorescent proteins include, but are not limited to, green fluorescent protein (GFP), enhanced green fluorescent protein (EGFP), Emerald, Superfolder GFP, Azami Green, mWasabi, TagGFP, TurboGFP, msGFP2, AcGFP, ZsGreen, T-Saphhire, BFP, EBFP, EBFP2, Azurite, mTagBFP, ECFP, ECFP, Cerulean, mTurquiose, CyPet, AmCyan1, Midori-Ishi Cyan, TagCFP, mTFP1 (Teal), EYFP, Topaz, Venus, mCitrine, YPet, TagYFP, PhiYFP, ZsYellow1, mBanana, Kusabira Orange, Kusabira Orange2, mOrange, mOrange2, tdTomato, dTomato-Tandem, TagRFP, TagRFP-T, DsRed, DsRed2, DsRed-Express (T1), DeRed-Monomer, mTangerine, mRuby, mApple, mStrawberry, Dendra 2, AsRed2, mRFP2, mRFP1, JRed, mCherry, mGreenLantem, HcRedl, mRaspberry, dKeima-Tandem, HcRed-Tandem, mPlum, AQ143, and the like.

In some embodiments, the fluorescent protein is a destabilized fluorescent protein. For example, any of the fluorescent proteins described herein, for example, GFP, can be linked to or tagged with a proline-glutamate-serine-threonine-rich (PEST) sequence from the mouse omithine decarboxylase gene to generate a destabilized fluorescent protein that has a reduced half-life as compared to a fluorescent protein that is not tagged with the PEST sequence. See, for example, Li et al. (Generation of destabilized green fluorescent protein as a transcription reporter. J Biol Chem. 1998; 273(52):34970-5). An exemplary nucleic acid sequence encoding GFP-PEST is provided herein as SEQ ID NO: 48. SEQ ID NO: 48 encodes a GFP-PEST polypeptide (SEQ ID NO: 49)

In some embodiments, the expression system is a m⁶A sensor system that expresses 1) a fusion protein in which an N⁶-methyladenosine (m⁶A) binding domain of a YT521-B homology (YTH) domain-containing protein is fused to a catalytic domain of a cytidine deaminase or a catalytic domain of an adenosine deaminase, and 2) a reporter mRNA which encodes GFP as a readout for the presence of m⁶A (FIG. 1). In this example, the reporter mRNA includes, in the following order, the coding sequence for enhanced GFP (EGFP), followed by a short exemplary m⁶A “sensor sequence” (5′GACUUACGACAG3′) (SEQ ID NO: 16), which contains two m⁶A consensus motifs (GAC) and two tandem “convertible” stop codon sequences that are in-frame with EGFP (FIG. 1). When unedited, the convertible stop codons encode arginine and glutamine (CGA and CAG, respectively). However, C-to-U editing produces two stop codons (UGA and UAG) (FIG. 1). Downstream of the m⁶A sensor sequence and in-frame with EGFP is the coding sequence for a destabilization domain modified from the Escherichia coli dihydrofolate reductase gene (DHFR). In some embodiments, the nucleic acid sequence encoding DHFR comprises SEQ ID NO: 36. This DHFR destabilization domain induces rapid, proteasome-mediated degradation of proteins to which it is tethered. Thus, when the GFP-DHFR m⁶A reporter mRNA is introduced into cells together with the fusion protein (e.g., APO1-YTH), if the reporter mRNA is not methylated, there will be no editing of the m⁶A sensor sequence by APO1-YTH and the full-length GFP-DHFR protein will be translated. The result is rapid degradation of GFP-DHFR and no fluorescence (FIG. 1, left panel). However, if either of the GAC sequences within the m⁶A sensor sequence is methylated, APO1-YTH will bind to the m⁶A and deaminate one or both cytidine residues within the two convertible stop codons of the sensor sequence. The result is translation of GFP followed by translation termination before the ribosome encounters the DHFR sequence. The GFP protein will not be degraded since it will not be fused to DHFR, resulting in GFP fluorescence (FIG. 1, right panel). Thus, this system provides a simple fluorescent readout for the presence of m⁶A (i.e., no m⁶A=no GFP fluorescence; m⁶A=GFP fluorescence).

### III. Cells and Transgenic Animals

Aspects of this disclosure include host cells and transgenic animals comprising the nucleic acid sequences or constructs described herein as well as methods of making such cells and transgenic animals.

A host cell comprising a nucleic acid or a vector described herein is provided. The host cell can be an in vitro, ex vivo, or in vivo host cell. Populations of any of the host cells described herein are also provided. A cell culture comprising one or more host cells described herein is also provided. Methods for the culture and production of many cells, including cells of bacterial (for example E. coli and other bacterial strains), animal (especially mammalian), and archebacterial origin are available in the art. See e.g., Sambrook, Ausubel, and Berger (all supra), as well as Freshney (1994) Culture of Animal Cells, a Manual of Basic Technique, 3rd Ed., Wiley-Liss, New York and the references cited therein; Doyle and Griffiths (1997) Mammalian Cell Culture: Essential Techniques John Wiley and Sons, NY; Humason (1979) Animal Tissue Techniques, 4th Ed. W.H. Freeman and Company; and Ricciardelli, et al., (1989) In vitro Cell Dev. Biol. 25:1016-1024.

The host cell can be a prokaryotic cell, including, for example, a bacterial cell. Alternatively, the cell can be a eukaryotic cell, for example, a mammalian cell. In some embodiments, the cell can be an HEK293T cell, a Chinese hamster ovary (CHO) cell, a COS-7 cell, a HELA cell, an avian cell, a myeloma cell, a Pichia cell, an insect cell or a plant cell. A number of other suitable host cell lines have been developed and include myeloma cell lines, fibroblast cell lines, and a variety of tumor cell lines such as melanoma cell lines. The vectors containing the nucleic acid segments of interest can be transferred or introduced into the host cell by well-known methods, which vary depending on the type of cellular host.

As used herein, the phrase“introducing” in the context of introducing a nucleic acid into a cell refers to the translocation of the nucleic acid sequence from outside a cell to inside the cell. In some cases, introducing refers to translocation of the nucleic acid from outside the cell to inside the nucleus of the cell. Various methods of such translocation are contemplated, including but not limited to, electroporation, nanoparticle delivery, viral delivery, contact with nanowires or nanotubes, receptor mediated internalization, translocation via cell penetrating peptides, liposome mediated translocation, DEAE dextran, lipofectamine, calcium phosphate or any method now known or identified in the future for introduction of nucleic acids into prokaryotic or eukaryotic cellular hosts. A targeted nuclease system (e.g., an RNA-guided nuclease, a transcription activator-like effector nuclease (TALEN), a zinc finger nuclease (ZFN), or a megaTAL (MT) (Li et al. Signal Transduction and Targeted Therapy 5, Article No. 1 (2020)) can also be used to introduce a nucleic acid, for example, a nucleic acid encoding a fusion protein and/or mRNA transcript (e.g, mRNA reporter mRNA) described herein, into a host cell

The CRISPR/Cas9 system, an RNA-guided nuclease system that employs a Cas9 endonuclease, can be used to edit the genome of a host cell or organism. The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, for example, Cas9, in complex with guide and activating RNA to recognize and cleave foreign nucleic acid. Guide RNAs having the activity of both a guide RNA and an activating RNA are also known in the art. In some cases, such dual activity guide RNAs are referred to as a single guide RNA (sgRNA).

As used herein, the term “Cas9” refers to an RNA-mediated nuclease (e.g., of bacterial or archeal orgin, or derived therefrom). Exemplary RNA-mediated nucleases include the foregoing Cas9 proteins and homologs thereof. Other RNA-mediated nucleases include Cpf1 (See, e.g., Zetsche et al., Cell, Volume 163, Issue 3, p759-771, 22 Oct. 2015) and homologs thereof.

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep. 24; 110(39):15644-9; Sampson et al., Nature. 2013 May 9;497(7448):254-7; and Jinek, et al., Science. 2012 Aug. 17; 337(6096):816-21. Variants of any of the Cas9 nucleases provided herein can be optimized for efficient activity or enhanced stability in the host cell. Thus, engineered Cas9 nucleases are also contemplated. See, for example, “Slaymaker et al., “Rationally engineered Cas9 nucleases with improved specificity,” Science 351 (6268): 84-88 (2016)).

Any of the components encoded by the nucleic acid constructs described herein, for example, fusion proteins or a m⁶A reporter mRNA, can be purified or isolated from a host cell or population of host cells. For example, a recombinant nucleic acid encoding any of the fusion proteins described herein can be introduced into a host cell under conditions that allow expression of the fusion protein. In some embodiments, the recombinant nucleic acid is codon-optimized for expression. After expression in the host cell, the fusion protein can be isolated or purified. Similarly, any of the nucleic acids encoding a m⁶A reporter mRNA described herein can be introduced into a host cell under conditions that allow transcription of the m⁶A reporter mRNA. After expression in the host cell, the m⁶A reporter mRNA can be isolated or purified.

Also provided is a non-human transgenic animal comprising a mammalian host cell that comprises any of the nucleic acid sequences or constructs described herein. Methods for making transgenic animals, include, but are not limited to, oocyte pronuclear DNA microinjection, intracytoplasmic sperm injection, embryonic stem cell manipulation, somatic nuclear transfer, recombinase systems (for example, Cre-LoxP systems, Flp-FRT systems and others), zinc finger nucleases (ZNFs), transcriptional activator-like effector nucleases (TALENs) and clustered regularly interspaced short palindromic repeat/CRISPR-associated protein 9 (CRISPR/Cas9). See, for example, Volobueva et al. Braz. J. Med. Biol. Res. 52(5): e8108 (2019)).

The term “transgenic animal” as used herein means an animal into which a genetic modification has been introduced by a genetic engineering procedure and in particular an animal into which has been introduced an exogenous nucleic acid. That is the animal comprises a nucleic acid sequence which is not normally present in the animal. Included are both progenitor and progeny animals. Progeny animals include animals which are descended from the progenitor as a result of sexual reproduction or cloning and which have inherited genetic material from the progenitor. Thus, the progeny animals comprise the genetic modification introduced into the parent. A transgenic animal may be developed, for example, from embryonic cells into which the genetic modification (e.g. exogenous nucleic acid sequence) has been directly introduced or from the progeny of such cells. The exogenous nucleic acid is introduced artificially into the animal (e.g. into a founder animal). Animals that are produced by transfer of an exogenous nucleic acid through breeding of the animal comprising the nucleic acid (into whom the nucleic acid was artificially introduced), which are progeny animals, are also included. Representative examples of non-human mammals include, but are not limited to non-human primates, mice, rats, rabbits, pigs, goats, sheep, horses, zebrafish and cows. A cell or a population of cells from any of the non-human transgenic animals provided herein is also provided.

The exogenous nucleic acid may be integrated into the genome of the animal or it may be present in an non-integrated form, e.g. as an autonomously-replicating unit, for example, an artificial chromosome which does not integrate into the genome, but which is maintained and inherited substantially stably in the animal. In some embodiments, the exogenous nucleic acid is under the control of a cell-specific or tissue-specific promoter. For example, transgenic animals that express a fusion protein and a mRNA reporter sequence in specific cells or tissues can be produced by introducing one or more nucleic acids into fertilized eggs, embryonic stem cells or the germline of the animal, wherein the one or more nucleic acids are under the control of a specific promoter which allows expression of the nucleic acid fusion protein and mRNA reporter sequence in specific types of cells or tissues. As used herein, a protein or mRNA is expressed predominantly in a given tissue, cell type, cell lineage or cell, when 90% or greater of the observed expression occurs in the given tissue cell type, cell lineage or cell.

In some embodiments, the exogenous nucleic acid in the animal is under the control of a constitutive or an inducible promoter, as described above. Inducible systems can also be used to allow expression of the fusion and/or mRNA reporter sequence at designated times during development, expanding the temporal specificity of fusion protein and/or mRNA reporter expression in the transgenic animal.

### IV. Methods

This disclosure also provides methods for detecting detecting m⁶A methylation in live cells. The methods according to the present disclosure substantially improve the time and cost associated with m⁶A detection while avoiding isolation of RNA from cells.

Provided herein is a method for detecting m⁶A methylation-dependent expression of a heterologous polypeptide in one or more cells comprising: a) introducing any of the expression systems described herein into one or more cells; and detecting expression of the heterologous protein, wherein expression of the heterologous protein is indicative of m⁶A methylation-dependent expression of the heterologous polypeptide in the one or more cells. As set forth above, when any of the expression systems described herein is introduced into a cell, if m⁶A methylation occurs in the cell, the sensor mRNA expressed by the expression system, i.e., a mRNA comprising a heterologous protein, a m⁶A sensor sequence and a destabilization domain (e.g., DHFR), will be methylated (at the m⁶A sensor sequence). Upon methylation, C to U editing results in a stop codon in the m⁶A sensor sequence that inhibits expression of DHFR, thus allowing the heterologous protein to be expressed without degradation.

Expression of the heterologous protein can be detected using any means known in the art for example, antibody detection, PCR amplification, sequencing, etc. In some embodiments, the heterologous polypeptide comprises a tag that can be detected, for example, by an antibody, or used for purification of the heterologous polypeptide from the one or more cells. In some embodiments, the heterologous polypeptide comprises a selectable marker that can be used to detecting the presence of the heterologous polypeptide in the cell. Any of the methods provided herein can further include quantitating the amount of the heterologous polypeptide expressed in the cell. Such methods are well known in the art and include but are not limited to Western blots, immunohistochemistry, ELISA, immunoprecipitation, immunofluorescence, flow cytometry, immunocytochemistry, mass spectrometric analyses, e.g., MALDI-TOF and SELDI-TOF.

Also provided is a method for detecting m⁶A methylation in one or more cells comprising (a) introducing any of the reporter expression systems described herein into one or more cells; and (b) detecting expression of the reporter protein.

Also provided is a method for detecting in vitro m⁶A methylation in one or more cells comprising: (a) contacting one or more cells with (i) a fusion protein comprising an N⁶-methyladenosine (m⁶A) binding domain of a YT521-B homology (YTH) domain-containing protein fused to a catalytic domain of a cytidine deaminase or a catalytic domain of an adenosine deaminase; and (ii) a DNA construct comprising a nucleic acid sequence encoding a reporter protein; a m⁶A sensor sequence; and a polypeptide encoding dihydrofolate reductase (DHFR); and (b) detecting expression of the reporter protein in the one or more cells.

Also provided is a method for detecting in vitro m⁶A methylation in one or more cells comprising: (a) introducing into one or more cells with (i) a fusion protein comprising an N⁶-methyladenosine (m⁶A) binding domain of a YT521-B homology (YTH) domain-containing protein fused to a catalytic domain of a cytidine deaminase or a catalytic domain of an adenosine deaminase; and (ii) a mRNA sequence encoding a reporter protein, a m⁶A sensor sequence, and a polypeptide encoding dihydrofolate reductase (DHFR); and (b) detecting expression of the reporter protein in the one or more cells.

Further provided is a method for identifying an agent that modulates m⁶A methylation in a cell comprising: (a) contacting one or more cells comprising a reporter expression system described herein with an agent; and (b) detecting expression of the reporter protein in the one or more cells, wherein a decrease in expression of the reporter protein as compared to expression of the reporter protein in the absence of the agent indicates the agent decreases m⁶A methylation in the one or more cells, and wherein an increase in expression of the reporter protein as compared to expression of the reporter protein in the absence of the agent indicates the agent increases m⁶A methylation in the one or more cells.

Also provided is a method for identifying an agent that inhibits METTL3-dependent methylation in a cell comprising: (a) contacting one or more cells comprising a m⁶A reporter expression system described herein with an agent; (b) detecting expression of the reporter protein in the one or more cells, wherein a decrease in expression of the reporter protein as compared to expression of the reporter protein in the absence of the agent indicates the agent inhibits METTL3-dependent methylation in the one or more cells.

In some embodiments, the agent is a small molecule (e.g., a drug), a polypeptide (for example, a protein or a peptide), a nucleic acid (e.g., a cDNA, or an inhibitory RNA (e.g., siRNA, shRNA, miRNA), a ribozyme, an sgRNA, etc.). In some embodiments, the method is a high throughput method wherein a plurality (e.g., two or more agents) are screened for their ability to modulate m⁶A methylation in a cell. As used herein the phrase “modulate m⁶A methylation” means a difference in m⁶A methylation as compared to a control cell(s). Modulation can be an increase or a decrease in m⁶A residues in one or more target RNAs in a cell (for example, in the sensor m⁶A sequence or in one or more cellular RNAs). A decrease or reduction in m⁶A methylation can be a 10%, 20%, 30%, 50%, 60%, 70%, 80%, 90%, or 100% decrease in m⁶A methylation. An increase in m⁶A methylation can be a 10%, 20%, 30%, 40%, 50%, 60%, 70%, 80%, 90%, 100%, 200%, 300%, 400% increase or greater. There can also be a difference in the pattern of m⁶A residues, for example, a change in the presence or absence of m⁶A residues at different locations in the RNA. For example, methylation may occur at one or more adenosine residues in the one or more target sequences at different locations as compared to a reference pattern. In another example, methylation may not occur at one or more adenosine residues in one or more target sequences as compared to a reference pattern.

Any of the m⁶A reporter systems described herein can be used in a CRISPR screen to identify one or more genes that are involved in m⁶A methylation in a cell. In a typical CRISPR screen a CRISPR guide RNA (gRNA) library is introduced in bulk into cells (for example cells comprising a m⁶A reporter system described herein), such that individual cells receive different gRNAs and are perturbed according to the gRNA received by the cell. These gRNAs can be delivered by lentiviral transduction and are integrated into the DNA of the target cells, making it possible to efficiently determine the induced perturbations based on the gRNA sequence. The CRISPR-Cas protein is either stably expressed in the cells or ectopically introduced as a plasmid, virus, mRNA or protein. Cells that exhibit decreased GFP expression (i.e. decreased methylation) as compared to GFP expression comprise a gRNA that disrupts a gene involved in m⁶A methylation in a cell. The gRNAs in these cells can be sequenced to determine the location of the gRNA insertion and thus identify the genes involved in m⁶A methylation. Cells that exhibit increased GFP expression (i.e. increased methylation) as compared to GFP expression in a control cell (for example, a cell that does not comprise a gRNA) comprise a gRNA that disrupts a gene comprising a demethylase or a gene that negatively regulates m⁶A methylation in a cell. The gRNAs in these cells can be sequenced to determine the location of the gRNA insertion and thus identify the genes involved in demethylation or negative regulation of m⁶A methylation.

In any of the methods described herein, the first DNA construct and/or second DNA construct can be stably or transiently expressed in an in vitro, ex vivo or in vivo cell. In some embodiments, the one or more cells are in vitro cells. In some embodiments, the one or more cells are one or more cells from a subject. In some embodiments, the one or more cells are in a subject.

Also provided is a method of detecting m⁶A methylation in a non-human transgenic animal comprising: generating a transgenic animal expressing the two components (i.e., the first DNA construct and the second DNA construct) of any m⁶A reporter system described herein; and detecting m⁶A methylation in the non-human transgenic animal.

Also provided is a method of identifying an agent that modulates m⁶A methylation in a non-human transgenic animal comprising contacting the non-human transgenic animal that expresses the two components (i.e., the first DNA construct and the second DNA construct) of any m⁶A reporter system described in this disclosure with an agent; and (b) detecting expression of the reporter protein in one or more cells of the non-human transgenic animal (e.g., cell samples, tissue samples, whole organism imaging), wherein a decrease in expression of the reporter protein as compared to expression of the reporter protein in the absence of the agent indicates the agent decreases m⁶A methylation in the non-human transgenic animal, and wherein an increase in expression of the reporter protein as compared to expression of the reporter protein in the absence of the agent indicates the agent increases m⁶A methylation in the non-human transgenic animal.

In some methods, viral-mediated delivery is used to introduce the m⁶A reporter system into the animal. In some methods, the m⁶A reporter system is introduced into the animal under the control of a cell-specific or tissue-specific promoter so that the first DNA construct and the second DNA construct of any m⁶A reporter system described is expressed in a desired tissue of interest to monitor the in vivo effects of m⁶A methylation in specific cells and/or tissues. Some embodiments further comprise administering an agent (e.g., a m⁶A methylation inhibitor) to determine its effects on methylation and/or for understanding tissue-specific differences in methylation. In some embodiments, the agent is a small molecule (e.g., a drug), a polypeptide (for example, a protein or a peptide), a nucleic acid (e.g., a cDNA, an inhibitory RNA (e.g., siRNA, shRNA, miRNA), a ribozyme an sgRNA, etc.). In some embodiments, cellular stress is applied to the animal, for example, heat stress, oxidative stress, nutrient stress, or genotoxic stress, to name a few, to determine how the stress affects methylation in one or more cells or tissues of the animal.

Any of the methods described herein can further comprise a) isolating RNA from the one or more in vitro, ex vivo or in vivo cells; b) amplifying one or more target sequences in the isolated RNA; and c) sequencing the mRNA comprising the m⁶A sensor sequence and/or one or more target RNA sequences to identify cytidine to uridine deamination at sites adjacent to one or more m⁶A residues, thus detecting the m⁶A residues in the RNA of the one of more cells. In some embodiments, the one or more RNA target sequences are amplified by reverse transcriptase polymerase chain reaction (RT-PCR). In some embodiments, the RNA comprises one or more RNAs selected from the group consisting of messenger RNA (mRNA), transfer RNA (tRNA), small nuclear RNA (snRNA), a regulatory RNA, a transfer-messenger RNA (tmRNA), ribosomal RNA (rRNA), microRNA (miRNA), long noncoding RNA (lncRNA) or circular RNA (circRNA).

In some embodiments, the RNA is isolated from a population of cells. In some embodiments, a population of cells is separated into individual compartments, for example, tissue culture wells, prior to isolation of RNA from single cells. In some embodiments the amount of isolated RNA used in the method is less than about 200 ng, 175 ng, 150 ng, 125 ng, 100 ng, 75 ng, 50 ng, 25 ng, 15 ng, 10 ng, 5 ng, 0.5 ng, 0.1 ng or 0.01 ng.

In any of the methods provided herein, the one or more cells can be prokaryotic or eukaryotic cells. In some embodiments, the eukaryotic cell is a mammalian cell, a plant cell or a yeast cell. In some embodiments, the cell is a primary cell. As used herein, the term “primary” in the context of a primary cell, for example, a primary stem cell, refers to a cell that has not been transformed or immortalized. Such primary cells can be cultured, sub-cultured, or passaged a limited number of times (e.g., cultured 0, 1, 2, 3, 4, 5, 6, 7, 8, 9, 10, 11, 12, 13, 14, 15, 16, 17, 18, 19, or 20 times). In some cases, the primary cells are adapted to in vitro culture conditions. In some cases, the primary cells are isolated from an organism, system, organ, or tissue, optionally sorted, and utilized directly without culturing or sub-culturing. In some cases, the primary cells are stimulated, activated, or differentiated. In some embodiments, the primary cells are neurons, brain cells or hematopoietic cells. In any of the methods described herein, the cell can be an in vitro, an ex vivo, or an in vivo cell.

In any of the methods described herein, the one or more target sequences can be amplified, for example, using reverse-transcriptase PCT (RT-PCR or RT-qPCR), to generate a cDNA that can be sequenced. In some embodiments, RNA-Seq is used for amplification and sequencing. In some embodiments, RNA-Seq is used for single cell sequencing or in situ sequencing of fixed tissue. See, Chu et al. “RNA sequencing: platform selection, experimental design, and data interpretation”. Nucleic Acid Therapeutics. 22 (4): 271-4 (2012); and Lee et al. “Highly multiplexed subcellular RNA sequencing in situ”. Science. 343 (6177): 1360-3 (2014). In some embodiments, targeted RNA-Seq is used for selecting and sequencing specific RNAs of interest. See, for example, Martin et al. “Targeted RNA Sequencing Assay to Characterize Gene Expression and Genomic Alterations,” J. Vis. Exp. 114: 54090 (2016).

Other sequencing methods that can be used to identify cytidine to uridine (thymidine in cDNA), or adenosine to inosine conversions include, but are not limited to, shotgun sequencing, bridge PCR, Sanger sequencing (including microfluidic Sanger sequencing), pyrosequencing, massively parallel signature sequencing, nanopore DNA sequencing, single molecule real-time sequencing (SMRT) (Pacific Biosciences, Menlo Park, CA), ion semiconductor sequencing, ligation sequencing, sequencing by synthesis (Illumina, San Diego, Ca), Polony sequencing, 454 sequencing, solid phase sequencing, DNA nanoball sequencing, heliscope single molecule sequencing, mass spectroscopy sequencing, pyrosequencing, Supported Oligo Ligation Detection (SOLiD) sequencing, DNA microarray sequencing, RNAP sequencing, tunneling currents DNA sequencing, and any other DNA sequencing method identified in the future. One or more of the sequencing methods described herein can be used in high throughput sequencing methods. As used herein, the term “high throughput sequencing” refers to all methods related to sequencing nucleic acids where more than one nucleic acid sequence is sequenced at a given time.

Any of the methods described herein can further comprise fixing the one or more cells and detecting cytidine to uridine deamination in the m⁶A sensor RNA sequence, and/or one or more target RNA sequences, wherein cytidine to uridine deamination is detected via mutation-sensitive in situ hybridization.

### Embodiments

1. An expression system comprising: (a) a first DNA construct comprising a nucleic acid sequence encoding a fusion protein, wherein the fusion protein comprises an N⁶-methyladenosine (m⁶A) binding domain of a YT521-B homology (YTH) domain-containing protein fused to a catalytic domain of a cytidine deaminase or a catalytic domain of an adenosine deaminase; and (b) a second DNA construct comprising: (i) a nucleic acid sequence encoding a heterologous polypeptide; (ii) a m⁶A sensor sequence; and (iii) a polypeptide encoding dihydrofolate reductase (DHFR).
2. The expression system of embodiment 1, wherein the m6A sensor sequence comprises SEQ ID NO: 16 (GACTTACGACAG).
3. The expression system of embodiment 1, wherein the m⁶A binding domain is fused to the catalytic domain via a peptide linker.
4. The expression system of embodiment 1 or embodiment 2, wherein the m⁶A binding domain comprises a polypeptide having at least 95% identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.
5. The expression system of any one of embodiments 1-4, wherein the catalytic domain comprises a polypeptide having at least 95% identity to SEQ ID NO 12 or a catalytic fragment thereof, SEQ ID NO: 13 or a catalytic fragment thereof; SEQ ID NO: 14 or a catalytic fragment thereof; or SEQ ID NO: 15.
6. The expression system of any one of embodiments 1-5, wherein a vector comprises the first DNA construct.
7. The expression system of any one of embodiments 1-5, wherein a vector comprises the second DNA construct.
8. The expression system of any one of embodiments 1-5, wherein a vector comprises the first DNA construct and the second DNA construct.
9. The expression system of any one of embodiments 1-8, wherein the nucleic acid sequence encoding a fusion protein and/or the nucleic acid sequence encoding a heterologous polypeptide and a polypeptide encoding dihydrofolate reductase (DHFR) are operably linked to a first promoter.
10. The expression system of embodiment 8 or 9, wherein the vector further comprises a nucleic acid sequence encoding a selectable marker operably linked to a second promoter.
11. The expression system of embodiment 10, wherein the selectable marker is a fluorescent protein.
12. The expression system of embodiment 11, wherein the fluorescent protein is dsRed.
13. The expression system of any of embodiments 9-12, wherein the promoter is a constitutive or an inducible promoter.
14. The expression system of any one of embodiments 1-13, wherein the cytidine deaminase is APOBEC-1.
15. The expression system of any one of embodiments 1-14, wherein the heterologous polypeptide is a reporter protein.
16. The expression system of embodiment 15, wherein the reporter protein is a fluorescent protein.
17. The expression system of embodiment 16, wherein the fluorescent protein is a green fluorescent protein.
18. A nucleic acid sequence comprising a nucleic acid sequence encoding a heterologous polypeptide, a m⁶A sensor sequence, and, a polypeptide encoding dihydrofolate reductase (DHFR).
19. A vector comprising the expression system of any one of embodiments 1-18.
20. A host cell comprising the expression system of any one of embodiments 1-17 or the vector of embodiment 19.
21. The host cell of embodiment 20, wherein the cell expresses a reporter protein that is not encoded by the first DNA construct or the second DNA construct.
22. A non-human transgenic animal comprising the host cell of embodiment 20 or 21.
23. A method for detecting m⁶A methylation-dependent expression of a heterologous polypeptide in one or more cells comprising: (a) introducing the expression system of any one of embodiments 1-17 into one or more cells; (b) detecting expression of the heterologous protein, wherein expression of the heterologous protein is indicative of m⁶A methylation-dependent expression of the heterologous polypeptide in the one or more cells.
24. A method for detecting m⁶A methylation in one or more cells comprising (a) introducing the expression system of any one of embodiments 15-17 into one or more cells; and (b) detecting expression of the reporter protein.
25. A method for detecting in vitro m6A methylation in one or more cells comprising (a) contacting one or more cells with (i) a fusion protein comprising an N⁶-methyladenosine (m⁶A) binding domain of a YT521-B homology (YTH) domain-containing protein fused to a catalytic domain of a cytidine deaminase or a catalytic domain of an adenosine deaminase; and (ii) a DNA construct comprising a nucleic acid sequence encoding a heterologous polypeptide, a m⁶A sensor sequence, and a polypeptide encoding dihydrofolate reductase (DHFR); and (b) detecting expression of the reporter protein.
26. A method for identifying an agent that modulates m6A methylation in a cell comprising: (a) contacting one or more cells comprising the reporter protein expression system of one of embodiments 15-17 with an agent; and (b) detecting expression of the reporter protein, wherein a decrease in expression of the reporter protein as compared to expression of the reporter protein in the absence of the agent indicates the agent decreases m⁶A methylation in the one or more cells, and wherein an increase in expression of the reporter protein as compared to expression of the reporter protein in the absence of the agent indicates the agent increases m⁶A methylation in the cell.
27. A method for identifying an agent that inhibits METTL3-dependent methylation in a cell comprising: (a) contacting one or more cells comprising the expression system of any one of embodiments 15-17 with an agent; (b) detecting expression of the reporter protein, wherein a decrease in expression of the reporter protein as compared to expression of the reporter protein in the absence of the agent indicates the agent inhibits METTL3-dependent methylation in the one or more cells.
28. The method of any one of embodiments 26-27, wherein the agent is a small molecule, a polypeptide or a nucleic acid.
29. The method of any one of embodiments 23-28, wherein the one or more cells are in vitro cells.
30. The method of any one of embodiments 23-28, wherein the cell is a cell from a subject.
31. The method of any one of embodiments 23-28 wherein the cell is in a subject.
32. A method for identifying an agent that modulates m⁶A methylation in a non-human transgenic animal comprising: (a) contacting a non-human transgenic animal that comprises the expression system of any one of embodiments 15-17 with an agent; (b) detecting expression of the reporter protein, wherein a decrease in expression of the reporter protein as compared to expression of the reporter protein in the absence of the agent indicates the agent decreases m⁶A methylation, and wherein an increase in expression of the reporter protein as compared to expression of the reporter protein in the absence of the agent indicates the agent increases m⁶A methylation.
33. A method for identifying an agent that inhibits METTL3-dependent methylation in a non-human transgenic animal comprising: (a) contacting a non-human transgenic animal that comprises the expression system of any one of embodiments 15-17 with an agent; and (b) detecting expression of the reporter protein, wherein a decrease in expression of the reporter protein as compared to expression of the reporter protein in the absence of the agent indicates the agent inhibits METTL3-dependent methylation.
34. The method of embodiment 32 or embodiment 33, wherein the expression system is expressed in a cell-specific or tissue-specific manner in the non-human transgenic animal.
35. The method of any one of embodiments 23-27, further comprising isolating RNA from the one or more cells, amplifying one or more target sequences in the RNA, and identifying cytidine to uridine deamination at sites adjacent to one or more m⁶A residues in the one or more target sequences.
36. The method of embodiment 34, wherein the one or more target sequences are amplified by reverse transcriptase polymerase chain reaction (RT-PCR).
37. The method of embodiment 34 or 36, wherein cytidine to uridine deamination is identified by sequencing the one or more target sequences.
38. The method of any one of embodiments 32-34, further comprising isolating RNA from one or more cells of the non-human transgenic animal, amplifying one or more target sequences in the RNA, and identifying cytidine to uridine deamination at sites adjacent to one or more m⁶A residues in the one or more target sequences.
39. The method of embodiment 38, wherein the one or more target sequences are amplified by reverse transcriptase polymerase chain reaction (RT-PCR).
40. The method of embodiment 38 or 39, wherein cytidine to uridine deamination is identified by sequencing the one or more target sequences.
41. A kit comprising the expression system of any one of embodiments 1-17.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### Examples

### Example 1. m⁶A Sensor Reporting System

As shown in FIG. 1, m⁶A reporter systems described herein can detect m⁶A in cells. First, APO1-YTH was expressed together with the m⁶A reporter RNA in HEK293T cells by co-transfecting separate plasmids encoding these two components of the reporter system. Cells expressing APO1-YTH together with the m⁶A reporter exhibited robust EGFP fluorescence, whereas cells expressing the m⁶A reporter alone showed only background levels of fluorescence (FIG. 1B). These results were accompanied by increased EGFP:EGFP-DHFR ratio as assayed by Western blot, as well as increased editing of the m⁶A reporter mRNA in cells transfected with the reporter and APO1-YTH compared to cells transfected with the reporter alone (FIGS. 1C and 1D). As an additional control, to demonstrate that this fluorescence is due to APO1-YTH recognition/editing near m⁶A in the reporter mRNA, cells were also co-transfected with the m⁶A reporter and APO1-YTH^{mut}, a version of the APO1-YTH fusion protein which lacks the m⁶A binding region of the YTH domain. This resulted in reduced fluorescence of the m⁶A reporter, decreased EGFP:EGFP-DHFR ratio by Western blot, and decreased m⁶A reporter editing (FIGS. 1B-1D). Together, these data indicate that m⁶A reporter fluorescence and editing are due to m⁶A recognition by APO1-YTH.

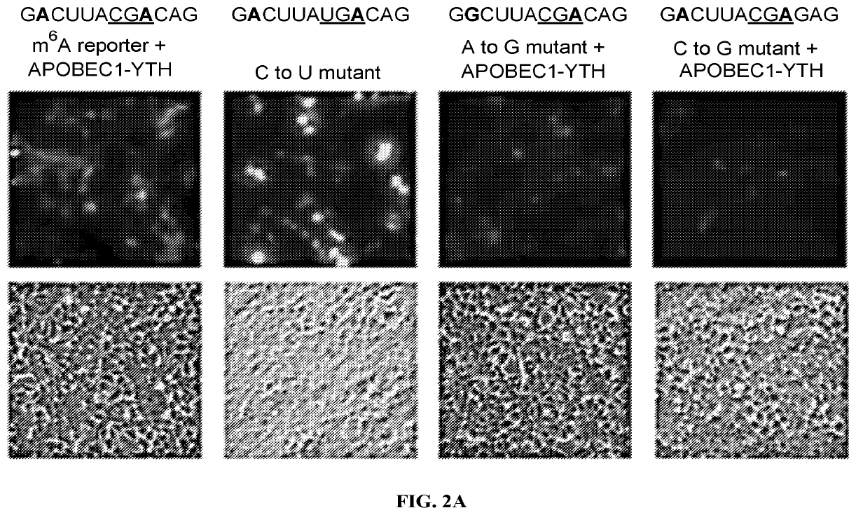

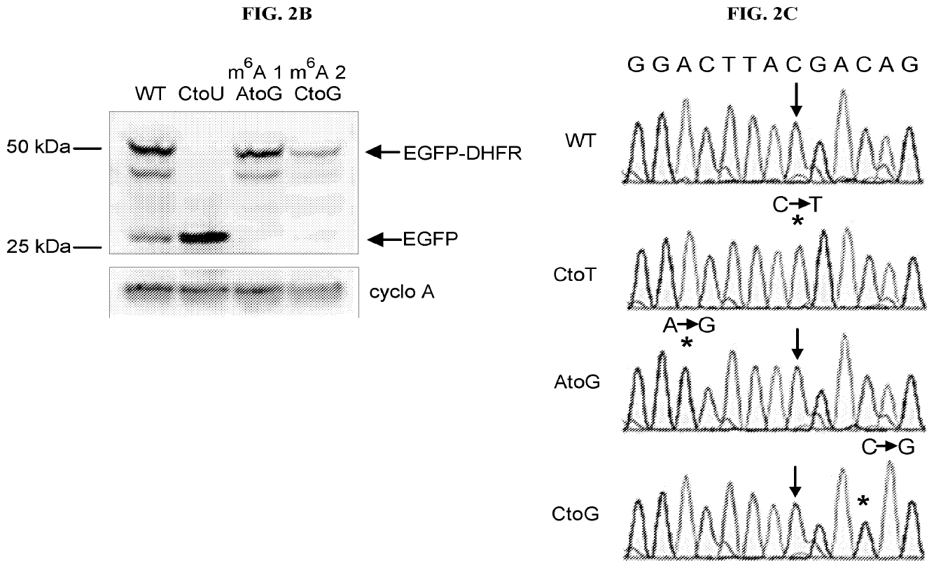

The m⁶A sites in the m⁶A reporter mRNA that are responsible for C-to-U editing were characterized. To do this, mutants of the m⁶A reporter were generated which blocked methylation of either the upstream or downstream GAC site in the sensor sequence. For the upstream site, the A was mutated to a G to preclude methylation. For the downstream site, because A to G mutation would result in the formation of a natural stop codon, instead the adjacent C was mutated to a G. This produces a GAG sequence instead of a GAC sequence, which would also preclude methylation since it destroys the m⁶A consensus. Finally, a positive control mutant reporter mRNA was generated in which the C was mutated within the CGACAG sequence of the sensor to a UGACAG sequence (C to T mutation in the plasmid DNA). This mimics the C to U editing of the original m⁶A reporter and produces a stop codon which is expected to result in 100% EGFP product and no EGFP-DHFR product (FIG. 2B).

Each of these m⁶A reporter constructs were co-transfected together with APO1-YTH into HEK293T cells. 24 h later, protein and RNA were collected and the cells were imaged. As expected, compared to the original m⁶A reporter, the C to T mutant plasmid showed a more robust EGFP signal and 100% EGFP protein product by Western blot (FIGS. 2A-2C).However, both the upstream m⁶A mutant reporter and the downstream m⁶A mutant reporter showed decreased EGFP fluorescence, decreased EGFP:EGFP-DHFR ratio, and decreased C to U editing compared to the original m⁶A reporter (FIGS. 2A-2C). These data indicate that both m⁶A sites, present in an upstream and a downstream GAC consensus sequence, contribute to the C to U editing at the CGA sequence between the two m⁶A sites. Thus, the m⁶A reporter utilizes two distinct m⁶A sites for generating the EGFP signal.

### Example 2. Stable Cell Lines Expressing APO1-YTH and the m6A Reporter

Another embodiment of the present disclosure provides stable cell lines that express the m⁶A reporter system as provided herein. To do this, stable cell lines expressing both the m⁶A reporter and APO1-YTH were generated by transfecting HEK293T cells with a dual-promoter plasmid encoding both components of the reporter system. This plasmid drives m⁶A reporter expression from the CMV promoter and APO1-YTH expression using an inducible EF1α promoter. However, any dual-promoter system could also be used and different promoters (inducible and constitutive) could also be used with this m⁶A reporter system. The m⁶A reporter system provided herein may also be applied in other mammalian and non-mammalian cell types as well.

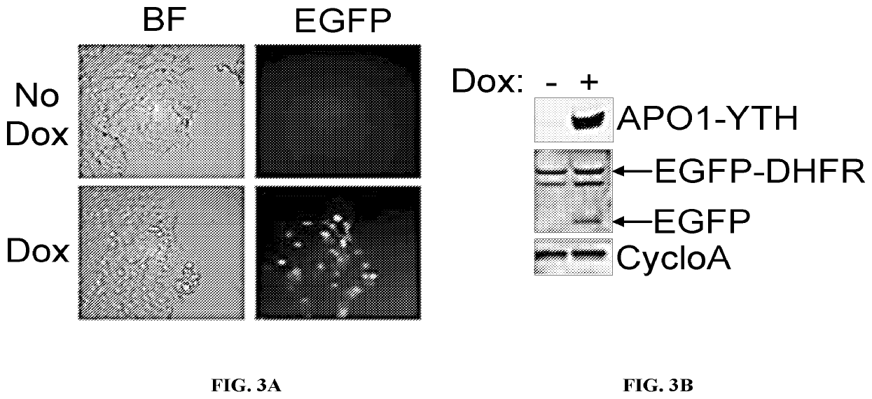

Following selection of stable cells with puromycin resistance which was conferred by genomic integration of the plasmid, it was next sought to confirm that the reporter system works in the context of stable integration of the m⁶A reporter and APO1-YTH components. HEK293T stable cell lines were treated with 1 ug/mL doxycycline (dox) and it was found that this led to induction of APO1-YTH protein as well as EGFP fluorescence (FIGS. 3A, 3B). This was not observed in cells treated with DMSO (vehicle control), indicating that there are low levels of background APO1-YTH and EGFP expression in these cells (FIGS. 3A, 3B). Various time courses were tested of dox induction and it was found that 24 h of dox treatment was sufficient for APO1-YTH induction and EGFP fluorescence. Altogether, these data show that stable cell lines can be used to express the m⁶A reporter together with APO1-YTH to detect m⁶A in cells. In some embodiments, the stable cell lines, such as those generated here, can be used in high-throughput screens for m⁶A inhibitors or in screens designed to identify proteins or other molecules that influence RNA methylation in cells (such as screens involving sgRNA libraries targeting endogenous cellular genes).

### Example 3. METLL3-dependent methylation

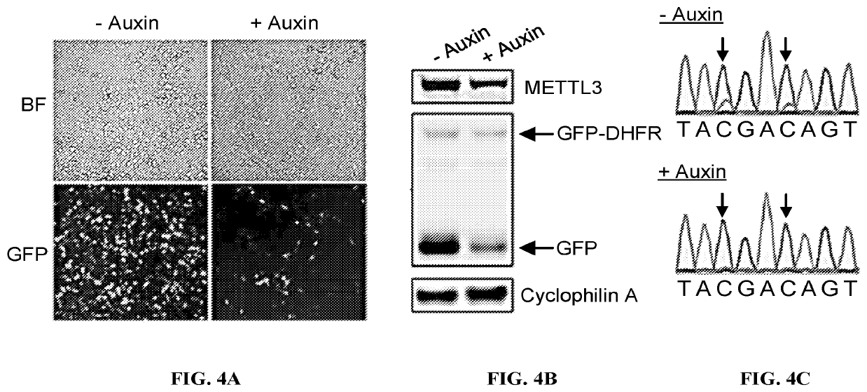

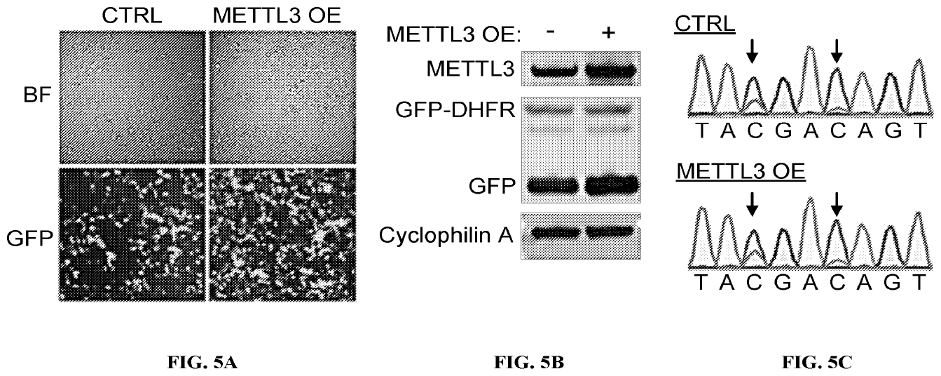

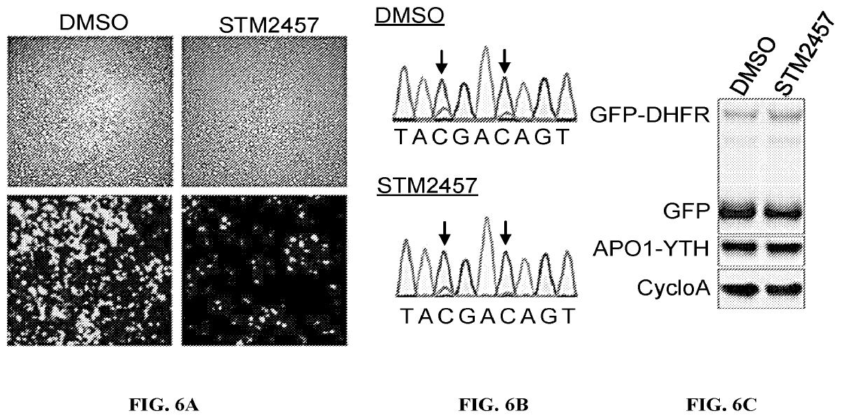

To confirm that the m⁶A sensor is METTL3-dependent, the m⁶A sensor system was expressed in HEK293T cells which contain an auxin-inducible degradation tag at the endogenous METTL3 locus (METTL3 degron cells). Cells were treated with auxin for 48 hours and then transfected with the m⁶A sensor system. 24 hours later, m⁶A sensor activity was assessed and it was found that GFP fluorescence is greatly diminished in auxin-treated cells compared to DMSO-treated cells (FIG. 4A). This is accompanied by a reduced GFP:GFP-DHFR ratio as assessed by western blot and decreased C-to-U editing of the m⁶A sensor sequence (FIG. 4B, 4C). Conversely, overexpression of METTL3 causes increased GFP fluorescence, a higher GFP:GFP-DHFR ratio, and increased C-to-U editing of the sensor sequence (FIGS. 5A-5C). To demonstrate that METTL3 methyltransferase activity is required for activation of the m⁶A sensor system, cells were treated with STM2457, a small molecule inhibitor of METTL313. Cells expressing the m⁶A sensor system exhibited reduced GFP fluorescence, reduced GFP protein, and lower levels of C-to-U editing of the sensor sequence after treatment with STM2457 (FIGS. 6A-6C). Altogether, these data show that activity of the m⁶A sensor system is METTL3-dependent and that the sensor responds both to genetic manipulation of METTL3 as well as small molecule inhibition of METTL3 activity.

### Example 4. Nonsense-Mediated Decay (NMD)

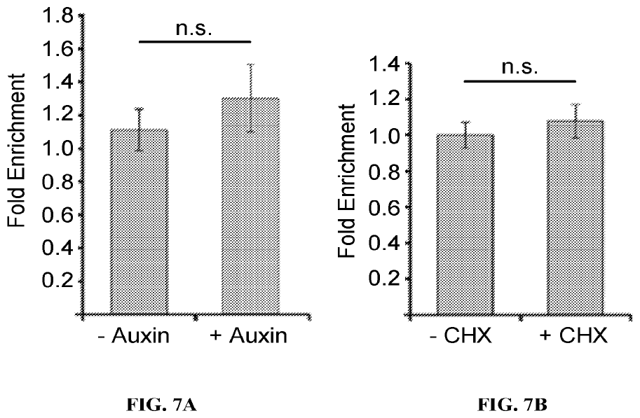

The possibility that the m⁶A reporter mRNA could be susceptible to nonsense-mediated decay (NMD), since editing of the m6A reporter mRNA produces premature stop codons, was investigated. However, this is unlikely since most NMD requires the presence of the exon junction complex (EJC), and the m⁶A reporter transcript lacks introns. Indeed, treatment of cells expressing the m⁶A sensor system with cycloheximide to block NMD did not cause changes in the levels of the m⁶A reporter mRNA, indicating that editing of the m⁶A reporter mRNA does not make it a target for NMD (FIG. 7B).

### Example 5. Background Fluorescence

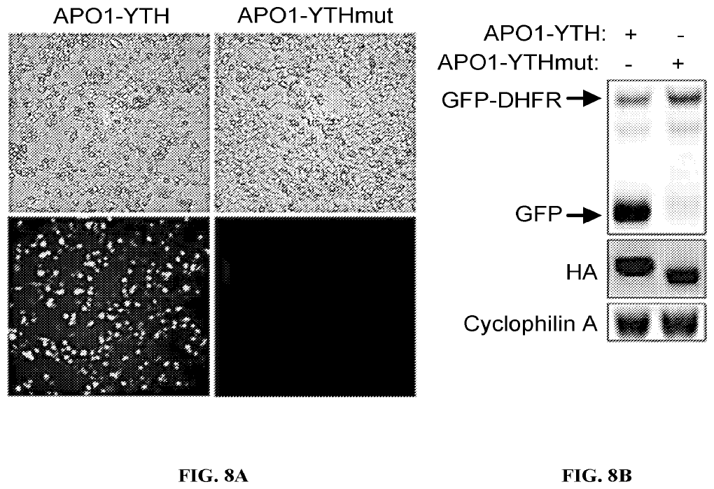

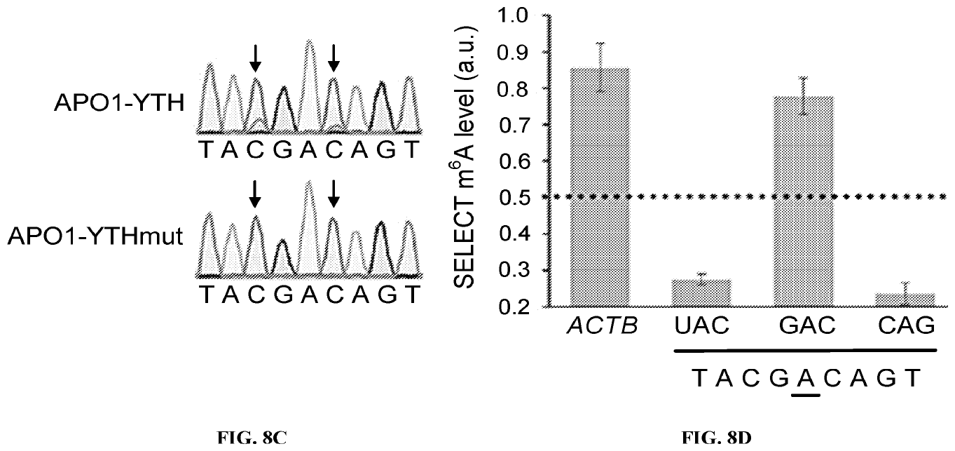

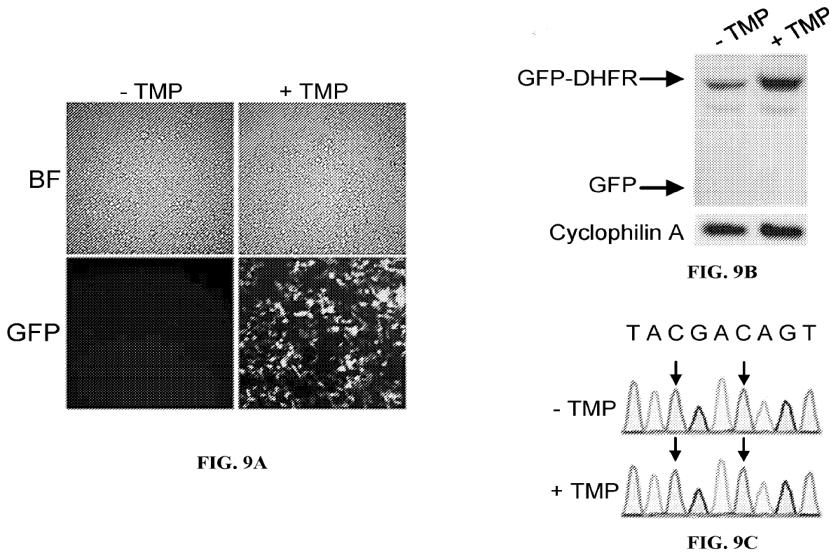

Next, the contribution of the GFP-DHFR protein to cellular fluorescence, which is a potential source of background signal in the system, was investigated Cells expressing the m⁶A reporter mRNA alone or together with APO1-YTHmut are dark, despite expressing the GFP-DHFR protein (FIGS. 8A-8B). This suggests that GFP-DHFR observed by Western blot reflects nascent protein that has not yet been degraded but which does not produce a fluorescent signal, presumably because it is destroyed before it can properly fold. To confirm that GFP-DHFR is not capable of producing fluorescence that could contribute to background signal in the sensor system, cells were transfected with the m⁶A reporter mRNA and treated with the small molecule trimethoprim (TMP), which binds to DHFR and blocks its ability to promote degradation. As expected, TMP treatment led to robust GFP-DHFR fluorescence and increased GFP-DHFR protein (FIG. 9A). Importantly, untreated cells remained dark (FIG. 9A). Together, these data indicate that GFP-DHFR does not contribute background fluorescence to the m⁶A sensor system and suggest that it is rapidly degraded before it can produce a fluorescent signal. This is consistent with previous studies using the DHFR degradation domain, which show rapid degradation of DHFR fusion proteins.

### Example 6. GFP-PEST

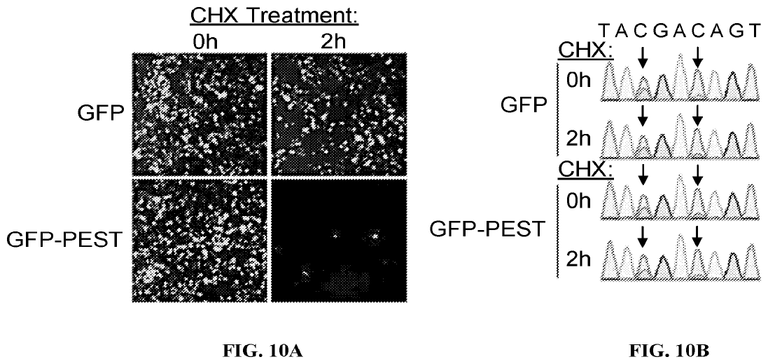

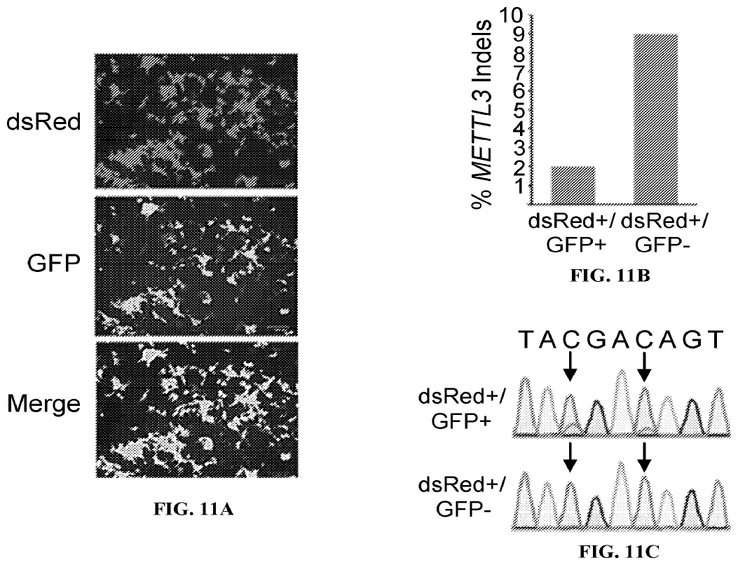

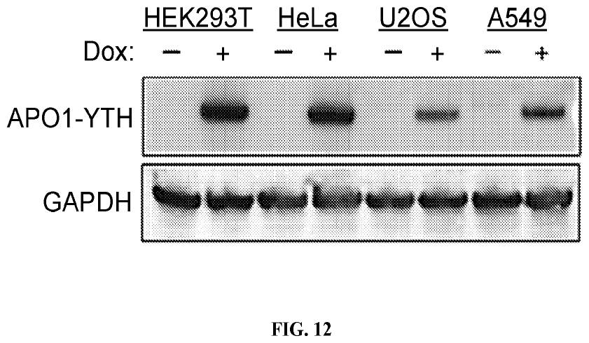

Another version of the m⁶A reporter mRNA, which encodes GFP fused to an optimized proline-glutamate-serine-threonine-rich (PEST) sequence from the mouse omithine decarboxylase gene (GFP-PEST), was made. This sequence is a commonly used tag for generating destabilized fusion proteins. See, for example, Li et al. Comparison of the GFP and GFP-PEST versions of the m⁶A sensor system revealed that GFP-PEST has a substantially reduced half-life while maintaining the same levels of C-to-U editing as the original (GFP) version (FIG. 10).

The m⁶A sensor has potential utility for a variety of applications, including as a readout for the effects of drugs/small molecules, genetic perturbations, or cellular conditions on m⁶A methylation. However, factors may influence pathways other than m⁶A that impact GFP fluorescence, such as transcription or translation, and therefore lead to a false readout. To help control for this, dsRed-Express229 (hereafter dsRed) was added to the m⁶A sensor system as an internal reporter under the control of a separate promoter (FIG. 11A). The ability of this system to detect changes in cellular m⁶A by conducting a modified knockout (KO) screen was examined. First, we HEK293T cells were infected with Cas9 and the Brunello CRISPR KO sgRNA library, which targets 19,114 genes in the human genome. A METTL3-targeting sgRNA was spiked in at a value of 10% of the total library. Cells underwent puromycin selection, followed by transfection with the improved m⁶A sensor system. 48 hours later, FACS was used to isolate cells based on red/green fluorescence. The sequence of the METTL3 locus was used to determine whether CRISPR-induced indels were enriched in dsRed+/GFP− cells, which would be expected if selective reduction of GFP fluorescence reflects METTL3 disruption. Indeed, it was found that METTL3 indels were nearly 5-fold higher in dsRed+/GFP− cells compared to dsRed+/GFP+ cells (FIG. 11B). This was accompanied by a decrease in C-to-U editing of the m⁶A sensor sequence, with nearly undetectable editing in the dsRed+/GFP− pool of cells (FIG. 11C). Together, these studies indicated that the red/green sensor system responds to METTL3 depletion. These studies demonstrate how the addition of an internal dsRed control can enable isolation of specific cell populations to better detect factors that reduce cellular m⁶A.

Collectively, these data demonstrate that the m⁶A sensor system provides a robust fluorescent readout for mRNA methylation in cells with minimal background. The activity of the m⁶Asensor is dependent on m⁶A binding by the APO1-YTH component of the system and that the sensor is responsive both to changes in METTL3 protein levels and inhibition of METTL3 activity. Variations of the system which include the use of GFP-PEST to improve the detection of m⁶A dynamics as well as an internal dsRed reporter to control for non-specific effects on fluorescent protein production, can also be used.

### Example 7. Demethylation

[0198]m⁶A can be demethylated by two known demethylase enzymes (“erasers”): ALKBH5 and FTO. However, these proteins target only a subset of methylated mRNAs in cells, and it is not clear in what contexts specific m6A residues are demethylated by each of these proteins. Thus, it is possible that the m6A reporter mRNA is targeted by one or both of these proteins. We will transfect FTO or ALKBH5 into HEK293T cells expressing the m⁶A sensor system and determine whether activity of the sensor is reduced over the course of 72 hours using fluorescence microscopy, Western blot, and Sanger sequencing at various timepoints as above. In addition, CRISPR/Cas9 will be used to knock out FTO and ALKBH5 and determine whether this enhances m6A sensor activity. Together, these studies will determine whether the m6A residues within the m6A sensor sequence are targeted by FTO or ALKBH5.

### Example 8. High Throughput Studies

Lentivirus expressing any of the m⁶A sensor systems described herein can be used to make stable HEK293T cell lines expressing the system. To avoid unwanted effects of prolonged APO1-YTH expression (which edits not only the m⁶A reporter mRNA but other methylated mRNAs in the cell as well), APO1-YTH will be expressed under an inducible promoter. Stable cell lines expressing APO1-YTH have been made and optimal doxycycline (dox) concentrations for maximal APO1-YTH protein production (FIG. 12) were determined. Methylation, editing, and GFP expression of these cells, in response to METTL3 depletion/overexpression, will be examined to ensure that the stable cell lines expressing the sensor system are sensitive to changes in m⁶A.

Once established, these m⁶A sensor cells will be used to develop assay conditions for using the m6A sensor system in HTS applications. The strategy will take advantage of the dsRed internal control by using FACS to sort distinct pools of cells based on GFP fluorescence relative to dsRed fluorescence. To develop the system, stable cells will be infected with Cas9 and METTL3 sgRNA-expressing lentivirus spiked into the Brunello library at 10% as described above (non-targeting sgRNA lentivirus will be spiked in as a control). Cells will then be selected with puromycin for 3 days, followed by dox treatment to induce APO1-YTH. 24-48 hours later, cells will be subjected to FACS to isolate target populations.

Four pools of cells will be isolated: dsRed+/GFP^{high} (GFP signal>1.5-fold higher than dsRed), dsRed+/GFP^{low} (GFP signal>1.5-fold lower than dsRed), dsRed+/GFP−, and unsorted cells. Sorting of the dsRed+/GFPhigh and dsRed+/GFPlow pools will be gated using a defined window of dsRed fluorescence to ensure that high and low GFP is specific to GFP and not due to general high and low FP production. For each pool, DNA will be isolated and the proportion of indels at the METTL3 locus will be measured. Each cell population will be compared in METTL3 sgRNA and control sgRNA spike-in experiments. The red/green gating strategy will be reviewed to find the optimal sorting conditions for capturing m⁶A- and METTL3-depleted cells. One goal is to achieve a 5-fold or greater increase in METTL3 indels in the dsRed+/GFP− and dsRed+/GFP^{low} populations compared to the unsorted and dsRed+/GFPhigh populations. Studies described above showed a 5-fold increase in dsRed+/GFP− cells compared to dsRed/GFP+ cells, but the use of stable cells compared to transfection, increased sensitivity of the optimized sensor system, and more stringent gating parameters could lead to higher indel enrichment in the dsRed+/GFP− and dsRed+/GFP^{low} populations compared to dsRed+/GFP^{high} cells and unsorted cells. It is possible that the greatest enrichment will be seen in the dsRed+/GFP− population of cells, but the dsRed+/GFP^{low} population will also be informative.

After using METTL3 indel analysis to optimize the sorting parameters, whether each population of cells reflects high/low sensor methylation and editing by isolating RNA and performing SELECT (see, for example, Zhang et al. “The detection and functions of RNA modification m6A based on m6A writers and erasers,” J. Biol. Chem. (2021)297(2): 100973) and RT-PCR/Sanger sequencing on the sensor sequence will be validated. Protein will be isolated and Western blot will be used to measure GFP/GFP-DHFR ratios and levels of METTL3, dsRed, and APO1-YTH.

To demonstrate the utility of the m⁶A sensor system for HTS applications, a negative selection-based global KO screen, designed to identify proteins that influence m⁶A methylation, will be conducted. The approach will be similar to the approach described above for determining HTS conditions, but cells will be infected with Cas9 and the Brunello sgRNA library only (no METTL3 sgRNA spike-in). Each gene is represented in this library by four independent sgRNAs to help control for off-target effects and minimize false-positive hits. After 3 days of puromycin selection, cells will be treated with dox and subjected to FACS.

Genomic DNA will be harvested from each pool of sorted cells, followed by amplification of sgRNA regions and next-generation sequencing. The number of reads for each sgRNA will be assessed in dsRed+/GFP− and dsRed+/GFPlow pools of cells relative to both the dsRed+/GFPhigh pool and the unsorted pool to identify genes whose disruption reduces m6A sensor activity. Statistically significant hits will be determined by the Duke Functional Genomics Core using MAGeCK38 and will involve ranking of individual sgRNAs based on their enrichment in each pool and prioritizing genes for which multiple sgRNAs are enriched. Genes involved in pathways that would be expected to impact GFP production from the reporter mRNA independent of m6A, such as those that influence general translation or APOBEC1 editing, will be filtered out from the final list of candidates. The results of these experiments will be the identification of a set of target genes whose reduction decreases activity of the m⁶A sensor.

In addition, sgRNA sequences enriched in the dsRed+/GFP^{high} pool of cells will be identified. These hits could reflect either m⁶A demethylase proteins or proteins that negatively regulate m⁶A methyltransferase activity. If FTO or ALKBH5 do not act on the m⁶A reporter mRNA, it is possible that other as-yet undiscovered m⁶A eraser proteins target these m⁶A residues, and those could be identified here.

Although several components of the m⁶A methyltransferase complex have been identified, the full complement of proteins that control m6A deposition has not been determined. Thus, although it is expected that HTS will identify core methyltransferase complex components such as METTL3, METTL14, and WTAP, it is also anticipated that other factors will be identified. However, it will be important to determine whether these other hits are m6A regulators or whether they are false-positives that impact the sensor system independent from influencing m6A.

The 5 hits from the screen that have not previously been identified as m⁶A methyltransferase complex proteins will be selected. To do this, CRISPR/Cas9 will be used to knock out each target gene in the m⁶A sensor stable cells. Target gene KO will be achieved using lentiviral infection of sgRNAs used in the global KO screen as well as at least one additional sgRNA to further eliminate non-specific effects. Validation of GFP reduction will then be done using fluorescence microscopy, western blot, and RT-PCR/Sanger sequencing of the m⁶A sensor sequence to confirm reduced GFP production and reduced C-to-U editing of the reporter mRNA. For targets that pass these tests, SELECT will be performed to confirm reduced methylation of the m⁶A sensor sequence. UPLC-MS/MS will be used to confirm loss of m⁶A in cellular mRNAs.

These studies could not only validate that the sensor system can identify known m⁶A regulatory proteins on a high-throughput platform, but also uncover genes/molecular pathways that lead to false-positive results. This knowledge will be important for further optimizing the system. For instance, if the dsRed+/GFP− pool is enriched for genes that control APO1-YTH induction, then constitutive APO1-YTH expression can be used instead.

Sequences:

SEQ ID NO: 1 (amino acid sequence of YTHDF2-YTH)
PHPVLEKLRSINNYNPKDFDWNLKHGRVFIIKSYSEDDIHRSIKYNIWCSTEHGNKRL
DAAYRSMNGKGPVYLLFSVNGSGHFCGVAEMKSAVDYNTCAGVWSQDKWKGRF
DVRWIFVKDVPNSQLRHIRLENNENKPVTNSRDTQEVPLEKAKQVLKIIASYKHTTSI
FDDFSHYEKRQEEEESVKKERQGRGK
SEQ ID NO: 2 (amino acid sequence of YTHDF2-YTH_W432A_W486A)
PHPVLEKLRSINNYNPKDFDWNLKHGRVFIIKSYSEDDIHRSIKYNIACSTEHGNKRL
DAAYRSMNGKGPVYLLFSVNGSGHFCGVAEMKSAVDYNTCAGVASQDKWKGRFD
VRWIFVKDVPNSQLRHIRLENNENKPVTNSRDTQEVPLEKAKQVLKIIASYKHTTSIF
DDFSHYEKRQEEEESVKKERQGRGK
SEQ ID NO: 3 (amino acid sequence of YTHDF2-YTHmut)
GRVFIIKSYSEDDIHRSIKYNIWCSTEHGNKRLDAAYRSMNGKGPVYLLFSVNGSGHF
CGVAEMKSAVDYNTCAGVWSQDKWKGRFDVRWIFVKDVPNSQLRHIRLENNENK
PVTNSRDTQEVPLEKAKQVLKIIASYKHTTSIFDDFSHYEKRQEEEESVKKERQGRGK
SEQ ID NO: 4 (amino acid sequence of YTHDF2-YTHmut2)
GRVFIIKSYSEDDIHRSIKYNIACSTEHGNKRLDAAYRSMNGKGPVYLLFSVNGSGHF
CGVAEMKSAVDYNTCAGVASQDKWKGRFDVRWIFVKDVPNSQLRHIRLENNENKP
VTNSRDTQEVPLEKAKQVLKIIASYKHTTSIFDDFSHYEKRQEEEESVKKERQGRGK
SEQ ID NO: 5 (amino acid sequence of YTHDF2-YTH D422N)
PHPVLEKLRSINNYNPKDFDWNLKHGRVFIIKSYSENDIHRSIKYNIWCSTEHGNKRL
DAAYRSMNGKGPVYLLFSVNGSGHFCGVAEMKSAVDYNTCAGVWSQDKWKGRF
DVRWIFVKDVPNSQLRHIRLENNENKPVTNSRDTQEVPLEKAKQVLKIIASYKHTTSI
FDDFSHYEKRQEEEESVKKERQGRGK
SEQ ID NO: 6 (amino acid sequence of YTHDF1)
HPVLEKLKAAHSYNPKEFEWNLKSGRVFIIKSYSEDDIHRSIKYSIWCSTEHGNKRLD
SAFRCMSSKGPVYLLFSVNGSGHFCGVAEMKSPVDYGTSAGVWSQDKWKGKFDVQ
WIFVKDVPNNQLRHIRLENNDNKPVTNSRDTQEVPLEKAKQVLKIISSYKHTTSIFDD
FAHYEKRQEEEEVVRKERQSRNKQ
SEQ ID NO: 7 (amino acid sequence of YTHDF1mut)
GRVFIIKSYSEDDIHRSIKYSIWCSTEHGNKRLDSAFRCMSSKGPVYLLFSVNGSGHFC
GVAEMKSPVDYGTSAGVWSQDKWKGKFDVQWIFVKDVPNNQLRHIRLENNDNKP
VTNSRDTQEVPLEKAKQVLKIISSYKHTTSIFDDFAHYEKRQEEEEVVRKERQSRNKQ
SEQ ID NO: 8 (amino acid sequence of YTHDF1 D401N)
HPVLEKLKAAHSYNPKEFEWNLKSGRVFIIKSYSEDNIHRSIKYSIWCSTEHGNKRLD
SAFRCMSSKGPVYLLFSVNGSGHFCGVAEMKSPVDYGTSAGVWSQDKWKGKFDVQ
WIFVKDVPNNQLRHIRLENNDNKPVTNSRDTQEVPLEKAKQVLKIISSYKHTTSIFDD
FAHYEKRQEEEEVVRKERQSRNKQ
SEQ ID NO: 9 (amino acid sequence of YTHDF3)
VHPVLEKLKAINNYNPKDFDWNLKNGRVFIIKSYSEDDIHRSIKYSIWCSTEHGNKRL
DAAYRSLNGKGPLYLLFSVNGSGHFCGVAEMKSVVDYNAYAGVWSQDKWKGKFE
VKWIFVKDVPNNQLRHIRLENNDNKPVTNSRDTQEVPLEKAKQVLKIIATFKHTTSIF
DDFAHYEKRQEEEEAMRRERNRNKQ
SEQ ID NO: 10 (amino acid sequence of YTHDC1)
SKLKYVLQDARFFLIKSNNHENVSLAKAKGVWSTLPVNEKKLNLAFRSARSVILIFSV
RESGKFQGFARLSSESHHGGSPIHWVLPAGMSAKMLGGVFKIDWICRRELPFTKSAH
LTNPWNEHKPVKIGRDGQEIELECGTQLCLLFPPDESIDLYQVIHKMRHK
SEQ ID NO: 11 (amino acid sequence of YTHDC2)
PVRYFIMKSSNLRNLEISQQKGIWSTTPSNERKLNRAFWESSIVYLVFSVQGSGHFQG
FSRMSSEIGREKSQDWGSAGLGGVFKVEWIRKESLPFQFAHHLLNPWNDNKKVQISR
DGQELEPLVGEQLLQLWERLPLGEKNTTD
SEQ ID NO: 12 (amino acid sequence of rAPOBEC1)
MSSETGPVAVDPTLRRRIEPHEFEVFFDPRELRKETCLLYEINWGGRHSIWRHTSQNT
NKHVEVNFIEKFTTERYFCPNTRCSITWFLSWSPCGECSRAITEFLSRYPHVTLFIYIAR
LY
HHADPRNRQGLRDLISSGVTIQIMTEQESGYCWRNFVNYSPSNEAHWPRYPHLWVR
LYVLELYCIILGLPPCLNILRRKQPQLTFFTIALQSCHYQRLPPHILWATGLK
SEQ ID NO: 13 (amino acid sequence of hAICDA)
MDSLLMNRRKFLYQFKNVRWAKGRRETYLCYVVKRRDSATSFSLDFGYLRNKNGC
HVELLFLRYISDWDLDPGRCYRVTWFTSWSPCYDCARHVADFLRGNPNLSLRIFTAR
LYFCEDRKAEPEGLRRLHRAGVQIAIMTFKDYFYCWNTFVENHERTFKAWEGLHEN
SVRLSRQLRRILLPLYEVDDLRDAFRTLGL
SEQ ID NO: 14 (amino acid sequence of hAPOBEC3A)
MEASPASGPRHLMDPHIFTSNFNNGIGRHKTYLCYEVERLDNGTSVKMDQHRGFLH
NQAKNLLCGFYGRHAELRFLDLVPSLQLDPAQIYRVTWFISWSPCFSWGCAGEVRAF
LQENTHVRLRIFAARIYDYDPLYKEALQMLRDAGAQVSIMTYDEFKHCWDTFVDHQ
GCPFQPWDGLDEHSQALSGRLRAILQNQGN
SEQ ID NO: 15 (amino acid sequence of catalytic domain of ADAR2)
QLHLPQVLADAVSRLVLGKFGDLTDNFSSPHARRKVLAGVVMTTGTDVKDAKVISV
STGTKCINGEYMSDRGLALNDCHAEIISRRSLLRFLYTQLELYLNNKDDQKRSIFQKS
ERGGFRLKENVQFHLYISTSPCGDARIFSPHEPILEEPADRHPNRKARGQLRTKIESGQ
GTIPVRSNASIQTWDGVLQGERLLTMSCSDKIARWNVVGIQGSLLSIFVEPIYFSSIILG
SLYHGDHLSRAMYQRISNIEDLPPLYTLNKPLLSGISNAEARQPGKAPNFSVNWTVG
DSAIEVINATTGKDELGRASRLCKHALYCRWMRVHGKVPSHLLRSKITKPNVYHES
KLAAKEYQAAKARLFTAFIKAGLGAWVEKPTEQDQFSLT
SEQ ID NO: 16 (m6A sensor sequence)
GACTTACGACAG
SEQ ID NO: 17-catalytic domain of ADAR2
MDSLLMNRREFLYQFKNVRWAKGRRETYLCYVVKRRDSATSFSLDFGYLRNKNGC
HVELLFLRYISDWDLDPGRCYRVTWFISWSPCYDCARHVADFLRGNPNLSLRIFTAR
LYFCEAGRREPEGLRRLHRAGVQIAIMTFKDYFYCWNTFVENHGRTFKAWEGLHEN
SVRLSRQLRRILL
SEQ ID NO: 18 (SGSETPGTSESATPE)
SEQ ID NO: 19 (SGSETPGTSESATPES)
SEQ ID NO: 20 ((GGGGS)₃)
SEQ ID NO: 21 ((GGGGS)₁₀),
SEQ ID NO: 22 (A(EAAAK)₃A)
SEQ ID NO: 23 (A(EAAAK)₁₀A)
SEQ ID NO: 24 (A(EAAAK)₂₀A)
SEQ ID NO: 25 E. coli codon optimized APOBEC1-YTH for protein
purification:
ATGAGCAGCGAAACCGGTCCGGTGGCGGTTGACCCGACCCTGCGTCGTCGTATT
GAGCCGCACGAGTTCGAAGTGTTCTTTGATCCGCGTGAGCTGCGTAAGGAAACCT
GCCTGCTGTACGAAATTAACTGGGGTGGCCGTCACAGCATCTGGCGTCACACCA
GCCAGAACACCAACAAGCACGTTGAGGTGAACTTCATCGAAAAATTTACCACCG
AGCGTTACTTCTGCCCGAACACCCGTTGCAGCATTACCTGGTTTCTGAGCTGGAG
CCCGTGCGGTGAATGCAGCCGTGCGATCACCGAGTTCCTGAGCCGTTATCCGCAC
GTTACCCTGTTTATCTACATTGCGCGTCTGTATCACCACGCGGACCCGCGTAACC
GTCAAGGTCTGCGTGATCTGATCAGCAGCGGCGTGACCATCCAGATTATGACCG
AGCAAGAAAGCGGTTACTGCTGGCGTAACTTCGTTAACTATAGCCCGAGCAACG
AAGCGCATTGGCCGCGTTACCCGCACCTGTGGGTGCGTCTGTACGTTCTGGAGCT
GTATTGCATCATTCTGGGCCTGCCGCCGTGCCTGAACATTCTGCGTCGTAAGCAG
CCGCAACTGACCTTCTTTACCATCGCGCTGCAGAGCTGCCACTACCAACGTCTGC
CGCCGCACATTCTGTGGGCGACCGGTCTGAAGAGCGGCAGCGAAACCCCGGGTA
CCAGCGAAAGCGCGACCCCGGAGCCGCACCCGGTGCTGGAGAAACTGCGTAGCA
TCAACAACTATAACCCGAAGGACTTCGATTGGAACCTGAAACACGGTCGTGTTTT
TATCATTAAGAGCTACAGCGAAGACGATATCCACCGTAGCATTAAATATAACAT
CTGGTGCAGCACCGAGCACGGCAACAAGCGTCTGGACGCGGCGTACCGTAGCAT
GAACGGTAAAGGCCCGGTGTATCTGCTGTTCAGCGTTAACGGTAGCGGCCACTTT
TGCGGTGTGGCGGAAATGAAAAGCGCGGTTGATTACAACACCTGCGCGGGTGTG
TGGAGCCAGGACAAGTGGAAAGGCCGTTTCGATGTTCGTTGGATTTTTGTGAAGG
ACGTTCCGAACAGCCAACTGCGTCACATCCGTCTGGAGAACAACGAAAACAAAC
CGGTGACCAACAGCCGTGATACCCAGGAAGTGCCGCTGGAAAAGGCGAAACAA
GTTCTGAAGATCATTGCGAGCTACAAACACACCACCAGCATCTTCGACGATTTTA
GCCACTATGAGAAGCGTCAGGAAGAGGAAGAGAGCGTGAAGAAGGAGCGTCAA
GGTCGTGGCAAACTGGAGTACCCGTATGACGTTCCGGATTATGCGTAAATTGGA
AGTGGATAA
SEQ ID NO: 26 E. coli codon optimized APOBEC1-YTHmut for protein
purification:
ATGAGCAGCGAAACCGGTCCGGTGGCGGTTGACCCGACCCTGCGTCGTCGTATT
GAGCCGCACGAGTTCGAAGTGTTCTTTGATCCGCGTGAGCTGCGTAAGGAAACCT
GCCTGCTGTACGAAATTAACTGGGGTGGCCGTCACAGCATCTGGCGTCACACCA
GCCAGAACACCAACAAGCACGTTGAGGTGAACTTCATCGAAAAATTTACCACCG
AGCGTTACTTCTGCCCGAACACCCGTTGCAGCATTACCTGGTTTCTGAGCTGGAG
CCCGTGCGGTGAATGCAGCCGTGCGATCACCGAGTTCCTGAGCCGTTATCCGCAC
GTTACCCTGTTTATCTACATTGCGCGTCTGTATCACCACGCGGACCCGCGTAACC
GTCAAGGTCTGCGTGATCTGATCAGCAGCGGCGTGACCATCCAGATTATGACCG
AGCAAGAAAGCGGTTACTGCTGGCGTAACTTCGTTAACTATAGCCCGAGCAACG
AAGCGCATTGGCCGCGTTACCCGCACCTGTGGGTGCGTCTGTACGTTCTGGAGCT
GTATTGCATCATTCTGGGCCTGCCGCCGTGCCTGAACATTCTGCGTCGTAAGCAG
CCGCAACTGACCTTCTTTACCATCGCGCTGCAGAGCTGCCACTACCAACGTCTGC
CGCCGCACATTCTGTGGGCGACCGGTCTGAAGAGCGGCAGCGAAACCCCGGGTA
CCAGCGAAAGCGCGACCCCGGAGGGTCGTGTTTTTATCATTAAGAGCTACAGCG
AAGACGATATCCACCGTAGCATTAAATATAACATCTGGTGCAGCACCGAGCACG
GCAACAAGCGTCTGGACGCGGCGTACCGTAGCATGAACGGTAAAGGCCCGGTGT
ATCTGCTGTTCAGCGTTAACGGTAGCGGCCACTTTTGCGGTGTGGCGGAAATGAA
AAGCGCGGTTGATTACAACACCTGCGCGGGTGTGTGGAGCCAGGACAAGTGGAA
AGGCCGTTTCGATGTTCGTTGGATTTTTGTGAAGGACGTTCCGAACAGCCAACTG
CGTCACATCCGTCTGGAGAACAACGAAAACAAACCGGTGACCAACAGCCGTGAT
ACCCAGGAAGTGCCGCTGGAAAAGGCGAAACAAGTTCTGAAGATCATTGCGAGC
TACAAACACACCACCAGCATCTTCGACGATTTTAGCCACTATGAGAAGCGTCAG
GAAGAGGAAGAGAGCGTGAAGAAGGAGCGTCAAGGTCGTGGCAAACTGGAGTA
CCCGTATGACGTTCCGGATTATGCGTAAATTGGAAGTGGATAA
SEQ ID NO: 27 (PKKKRKV)
SEQ ID NO: 28 (LPPLERLTL)
SEQ ID NO: 29 (MDPVVVLGLCLSCLLLLSLWKQSYGGG)
SEQ ID NO: 30 (METDTLLLWVLLLWVPGSTGD)
SEQ ID NO: 31 (EQKLISEEDL)
SEQ ID NO: 32 (GKPIPNPLLGLDST)
SEQ ID NO: 33 (IPNPLLGLD)
SEQ ID NO: 34 (DYKDDDDK)
SEQ ID NO: 35 (DYKDHDGDYKDHDIDYKDDDDK)
SEQ ID NO: 36 (DHFR domain)
ISLIAALAVDHVIGMETVMPWNLPADLAWFKRNTLNKPVIMGRHTWESIGRPLPGR
KNIILSSQPSTDDRVTWVKSVDEAIAACGDVPEIMVIGGGRVYEQFLPKAQKLYLTHI
DAEVEGDTHFPDYEPDDWESVFSEFHDADAQNSHSYCFEILERR
SEQ ID NO: 37
GACUUAUGACAG
SEQ ID NO: 38
GACUUACGACAG
SEQ ID NO: 39
GGACTTACGACAGTT
SEQ ID NO: 40
GACUUACGACAG
SEQ ID NO: 41
GACUUAUGACAG
SEQ ID NO: 42
GGCUUACGACAG
SEQ ID NO: 43
GACUUACGAGAG
SEQ ID NO: 44
HHHHHH
SEQ ID NO: 45 (Construct comprising a nucleic acid encoding GFP, a m⁶A reporter
sequence and DHFR; and a nucleic acid encoding APOBEC1-YTH (5′-3′)) 45
gtcgacggatcgggagatctcccgatcccctatggtgcactctcagtacaatctgctctgatgccgcatagttaagccagtatctgctcc
ctgcttgtgtgttggaggtcgctgagtagtgcgcgagcaaaatttaagctacaacaaggcaaggcttgaccgacaattgcatgaagaat
ctgcttagggttaggcgttttgcgctgcttcgcgatgtacgggccagatatacgcgttgacattgattattgactagttattaatagtaat
caattacggggtcattagttcatagcccatatatggagttccgcgttacataacttacggtaaatggcccgcctggctgaccgcccaacga
cccccgcccattgacgtcaataatgacgtatgttcccatagtaacgccaatagggactttccattgacgtcaatgggtggagtatttacgg
taaactgcccacttggcagtacatcaagtgtatcatatgccaagtacgccccctattgacgtcaatgacggtaaatggcccgcctggcatt
atgcccagtacatgaccttatgggactttcctacttggcagtacatctacgtattagtcatcgctattaccatggtgatgcggttttggca
gtacatcaatgggcgtggatagcggtttgactcacggggatttccaagtctccaccccattgacgtcaatgggagtttgttttggcaccaa
aatcaacgggactttccaaaatgtcgtaacaactccgccccattgacgcaaatgggcggtaggcgtgtacggtgggaggtctatataag
cagagctggtttagtgaaccgtcagatccgctagagatccgcggccgcgctagcgtttaaacgggccctctagagccgccatggtga
gcaagggcgaggagctgttcaccggggtggtgcccatcctggtcgagctggatggcgatgtaaatggccacaagttcagcgtgtcc
ggcgagggcgagggcgatgccacctacggcaagctcaccctgaagttcatctgcaccaccggcaagctgcccgtgccctggccca
ccctcgtcaccaccctcacctacggcgtgcagtgcttcagccgctaccccgatcacatgaagcagcacgatttcttcaagtccgccatg
cccgaaggctacgtccaggagcgcaccatcttcttcaaggatgatggcaattaccgtacccgcgccgaggtgaagttcgagggcgat
accctggtgaatcgcatcgagctgaagggcatcgatttcaaggaggatggcaatatcctggggcacaagctggagtacaattacaata
gccacaatgtctatatcatggccgataagcagaagaatggcatcaaggtgaatttcaagatccgccacaatatcgaggatggcagcgt
gcagctcgccgatcactaccagcagaatacccccatcggcgatggccccgtgctgctgcccgataatcactacctgagcacccagtc
cgccctgagcaaagatcccaatgagaagcgcgatcacatggtcctgctggagttcgtcaccgccgccgggatcactctcggcatgga
tgagctgtacaaggcggacttacgacagttgcgttacaccctttctcgacaaaacctaacttgcgcagaaaacatgccaatctcatcttg
gcttatcagtctgattgcggcgttagcggtagatcacgttatcggcatggaaaccgtcatgccgtggaacctgcctgccgatctcgcctg
gtttaaacgcaacaccttaaataaacccgtgattatgggccgccatacctgggaatcaatcggtcgtccgttgccaggacgcaaaaata
ttatcctcagcagtcaaccgagtacggacgatcgcgtaacgtgggtgaagtcggtggatgaagccatcgcggcgtgtggtgacgtac
cagaaatcatggttattggcggcggtcgcgtttatgaacagttcttgccaaaagcgcaaaaactgtatctgacgcatatcgacgcagaa
gtggaaggcgacacccatttcccggattacgagccggatgactgggaatcggtattcagcgaattccacgatgctgatgcgcagaact
ctcacagctattgctttgagattctggagcggcgataagcctcattgtgcattctctcgagtacccctacgacgtgcccgactacgcctga
gggacccgacaggcccgaaggaatagaagaagaaggtggagagagagacagagacagatccattcgattagtgaacggatcggc
actgcgtgcgccaattctgcagacaaatggcagtattcatccacaattttaaaagaaaaggggggattggggggtacagtgcagggg
aaagaatagtagacataatagcaacagacatacaaactaaagaattacaaaaacaaattacaaaaattcaaaattttcgggtttattacag
ggacagcagagatccagtttggttaccagtgtgatggatatctgcagaattcgcccttggatccgaattcctgcagccccgactttcactt
ttctctatcactgatagggagtggtaaactcgactttcacttttctctatcactgatagggagtggtaaactcgactttcacttttctcta
tcactgatagggagtggtaaactcgactttcacttttctctatcactgatagggagtggtaaactcgactttcacttttctctatcactga
tagggagtggtaaactcgactttcacttttctctatcactgatagggagtggtaaactcgactttcacttttctctatcactgatagggag
tggtaaactcgagggggatccactagcatgaagggcgaattccagcacactggtaacccgtgtcggctccagatctggcctccgcgccggg
ttttggcgcctcccgcgggcgcccccctcctcacggcgagccgcgttgacattgattattgactaggcttttgcaaaaagctttgcaaaga
tggataaagttttaaacagagaggaatctttgcagctaatggaccttctaggtcttgaaaggagtgggaattggctccggtgcccgtcagt
gggcagagcgcacatcgcccacagtccccgagaagttggggggaggggtcggcaattgaaccggtgcctagagaaggtggcgcgggg
taaactgggaaagtgatgtcgtgtactggctccgcctttttcccgaggggtggggagaaccgtatataagtgcagtagtcgccgtgaac
gttctttttcgcaacgggtttgccgccagaacacaggtaagtgccgtgtgtggttcccgcgggcctggcctctttacgggttatggccctt
gcgtgccttgaattacttccacctggctgcagtacgtgattcttgatcccgagcttcgggttggaagtgggtgggagagttcgaggcctt
gcgcttaaggagccccttcgcctcgtgcttgagttgaggcctggcctgggcgctggggccgccgcgtgcgaatctggtggcaccttc
gcgcctgtctcgctgctttcgataagtctctagccatttaaaatttttgatgacctgctgcgacgctttttttctggcaagatagtcttgt
aaatgcgggccaagatctgcacactggtatttcggtttttggggccgcgggcggcgacggggcccgtgcgtcccagcgcacatgttcggc
gaggcggggcctgcgagcgcggccaccgagaatcggacgggggtagtctcaagctggccggcctgctctggtgcctggcctcgc
gccgccgtgtatcgccccgccctgggcggcaaggctggcccggtcggcaccagttgcgtgagcggaaagatggccgcttcccggc
cctgctgcagggagctcaaaatggaggacgcggcgctcgggagagcggggggtgagtcacccacacaaaggaaaagggccttt
ccgtcctcagccgtcgcttcatgtgactccacggagtaccgggcgccgtccaggcacctcgattagttctcgagcttttggagtacgtc
gtctttaggttggggggaggggttttatgcgatggagtttccccacactgagtgggtggagactgaagttaggccagcttggcacttgat
gtaattctccttggaatttgccctttttgagtttggatcttggttcattctcaagcctcagacagtggttcaaagtttttttcttccattt
caggtgtcgtgaggaattagcttggtactaatacgactcactatagggagacccaagctggctaggtaagcttggtaccgagctcggatcc
actagtccagtgtggtggaattctgcagatatccagcacagtggggtttagtgaaccgtcagatccgctagagatccgcggccgctaatac
gactcactatagggagagccgccaccatgagctcagagactggcccagtggctgtggaccccacattgagacggcggatcgagcccc
atgagtttgaggtattcttcgatccgagagagctccgcaaggagacctgcctgctttacgaaattaattgggggggccggcactccattt
ggcgacatacatcacagaacactaacaagcacgtcgaagtcaacttcatcgagaagttcacgacagaaagatatttctgtccgaacac
aaggtgcagcattacctggtttctcagctggagcccatgcggcgaatgtagtagggccatcactgaattcctgtcaaggtatccccacg
tcactctgtttatttacatcgcaaggctgtaccaccacgctgacccccgcaatcgacaaggcctgcgggatttgatctcttcaggtgtgac
tatccaaattatgactgagcaggagtcaggatactgctggagaaactttgtgaattatagcccgagtaatgaagcccactggcctaggta
tccccatctgtgggtacgactgtacgttcttgaactgtactgcatcatactgggcctgcctccttgtctcaacattctgagaaggaagcag
ccacagctgacattctttaccatcgctcttcagtcttgtcattaccagcgactgcccccacacattctctgggccaccgggttgaaaagcg
gcagcgagactcccgggacctcagagtccgccacaccagaaccccacccagtgttggagaagcttcggtccattaataactataacc
ccaaagattttgactggaatctgaaacatggccgggttttcatcattaagagctactctgaggacgatattcaccgttccattaagtataa
tatttggtgcagcacagagcatggtaacaagagactggatgctgcttatcgttccatgaacgggaaaggccccgtttacttacttttcagt
gtcaacggcagtggacacttctgtggcgtggcagaaatgaaatctgctgtggactacaacacatgtgcaggtgtgtggtcccaggacaa
atggaagggtcgttttgatgtcaggtggatttttgtgaaggacgttcccaatagccaactgcgacacattcgcctagagaacaacgaga
ataaaccagtgaccaactctagggacactcaggaagtgcctctggaaaaggctaagcaggtgttgaaaattatagccagctacaagca
caccacttccatttttgatgacttctcacactatgagaaacgccaagaggaagaagaaagtgttaaaaaggaacgtcaaggtcgtggga
aactcgagtacccctacgacgtgcccgactacgcctgagtttaaaatcgatggtacactcgaggttaacgaattctaccgggtagggg
aggcgcttttcccaaggcagtctggagcatgcgctttagcagccccgctgggcacttggcgctacacaagtggcctctggcctcgcac
acattccacatccaccggtaggcgccaaccggctccgttctttggtggccccttcgcgccaccttctactcctcccctagtcaggaagtt
cccccccgccccgcagctcgcgtcgtgcaggacgtgacaaatggaagtagcacgtctcactagtctcgtgcagatggacagcaccg
ctgagcaatggaagcgggtaggcctttggggcagcggccaatagcagctttgctccttcgctttctgggctcagaggctgggaaggg
gtgggtccggggggggctcaggggcgggctcaggggggtggggggcccgaaggtcctccggaggcccggcattctgcac
gcttcaaaagcgcacgtctgccgcgctgttctcctcttcctcatctccgggcctttcgacctgcatcccgccaccatgaccgagtacaag
cccacggtgcgcctcgccacccgcgacgacgtccccagggccgtacgcaccctcgccgccgcgttcgccgactaccccgccacg
cgccacaccgtcgatccggaccgccacatcgagcgggtcaccgagctgcaagaactcttcctcacgcgcgtcgggctcgacatcgg
caaggtgtgggtcgcggacgacggcgccgcggtggcggtctggaccacgccggagagcgtcgaagcgggggcggtgttcgccg
agatcggcccgcgcatggccgagttgagcggttcccggctggccgcgcagcaacagatggaaggcctcctggcgccgcaccggc
ccaaggagcccgcgtggttcctggccaccgtcggagtctcgcccgaccaccagggcaagggtctgggcagcgccgtcgtgctccc
cggagtggaggcggccgagcgcgccggggtgcccgccttcctggagacctccgcgccccgcaacctccccttctacgagcggctc
ggcttcaccgtcaccgccgacgtcgaggtgcccgaaggaccgcgcacctggtgcatgacccgcaagcccggtgccggttccggcg
caacaaacttctctctgctgaaacaagccggagatgtcgaagagaatcctggaccgatggctagattagataaaagtaaagtgattaac
agcgcattagagctgcttaatgaggtcggaatcgaaggtttaacaacccgtaaactcgcccagaagctaggtgtagagcagcctacat
tgtattggcatgtaaaaaataagcgggctttgctcgacgccttagccattgagatgttagataggcaccatactcacttttgccctttaga
aggggaaagctggcaagattttttacgtaataacgctaaaagttttagatgtgctttactaagtcatcgcgatggagcaaaagtacattta
ggtacacggcctacagaaaaacagtatgaaactctcgaaaatcaattagcctttttatgccaacaaggtttttcactagagaatgcattat
atgcactcagcgctgtggggcattttactttaggttgcgtattggaagatcaagagcatcaagtcgctaaagaagaaagggaaacacctac
tactgatagtatgccgccattattacgacaagctatcgaattatttgatcaccaaggtgcagagccagccttcttattcggccttgaattg
atcatatgcggattagaaaaacaacttaaatgtgaaagtgggtcgccaaaaaagaagagaaaggtcgacggcggtggtgctttgtctcct
cagcactctgctgtcactcaaggaagtatcatcaagaacaaggagggcatggatgctaagtcactaactgcctggtcccggacactgg
tgaccttcaaggatgtatttgtggacttcaccagggaggagtggaagctgctggacactgctcagcagatcgtgtacagaaatgtgatg
ctggagaactataagaacctggtttccttgggttatcagcttactaagccagatgtgatcctccggttggagaagggagaagagccctg
gctggtgtaaagtagatgccgaccgaacaagagctgatttcgagaacgcctcagccagcaactcgcgcgagcctagcaaggcaaat
gcgagagaacggccttacgcttggtggcacagttctcgtccacagttcgctaagctcgctcggctgggtcgcgggagggccggtcgc
agtgattcaggcccttctggattgtgttggtccccagggcacgattgtcatgcccacgcactcgggtgatctgactgatcccgcagattg
gagatcgccgcccgtgcctgccgattgggtgcagatccgtcgagttaacaaaagaaaaggggggactggaagggctaattcactcc
caacgaagacaagatatcataacttcgtatagcatacattatacgaagttatcggctagctggtccggactgtactgggtctctctggtta
gaccagatctgagcctgggagctctctggctaactagggaacccactgcttaagcctcaataaagcttgccttgagtgcttcaagtagtg
tgtgcccgtctgttgtgtgactctggtaactagagatccctcagacccttttagtcagtgtggaaaatctctagcagggcccgtttaaacc
cgctgatcagcctcgactgtgccttctagttgccagccatctgttgtttgcccctcccccgtgccttccttgaccctggaaggtgccactc
ccactgtcctttcctaataaaatgaggaaattgcatcgcattgtctgagtaggtgtcattctattctggggggtggggtggggcaggacag
caagggggaggattgggaagacaatagcaggcatgctggggatgcggtgggctctatggcttctgaggcggaaagaaccagctgg
ggctctagggggtatccccacgcgccctgtagcggcgcattaagcgcggcgggtgtggtggttacgcgcagcgtgaccgctacactt
gccagcgccctagcgcccgctcctttcgctttcttcccttcctttctcgccacgttcgccggctttccccgtcaagctctaaatcgggggc
tccctttagggttccgatttagtgctttacggcacctcgaccccaaaaaacttgattagggtgatggttcacgtagtgggccatcgccctg
atagacggtttttcgccctttgacgttggagtccacgttctttaatagtggactcttgttccaaactggaacaacactcaaccctatctcg
gtctattcttttgatttataagggattttgccgatttcggcctattggttaaaaaatgagctgatttaacaaaaatttaacgcgaattaat
tctgtggaatgtgtgtcagttagggtgtggaaagtccccaggctccccagcaggcagaagtatgcaaagcatgcatctcaattagtcagca
accaggtgtggaaagtccccaggctccccagcaggcagaagtatgcaaagcatgcatctcaattagtcagcaaccatagtcccgcccctaa
ctccgcccatcccgcccctaactccgcccagttccgcccattctccgccccatggctgactaattttttttatttatgcagaggccgaggc
cgcctctgcctctgagctattccagaagtagtgaggaggcttttttggaggcctaggcttttgcaaaaagctcccgggagcttgtatatcc
attttcggatctgatcagcacgtgttgacaattaatcatcggcatagtatatcggcatagtataatacgacaaggtgaggaactaaaccat
ggccaagttgaccagtgccgttccggtgctcaccgcgcgcgacgtcgccggagcggtcgagttctggaccgaccggctcgggttctccc
gggacttcgtggaggacgacttcgccggtgtggtccgggacgacgtgaccctgttcatcagcgcggtccaggaccaggtggtgccg
gacaacaccctggcctgggtgtgggtgcgcggcctggacgagctgtacgccgagtggtcggaggtcgtgtccacgaacttccggga
cgcctccgggccggccatgaccgagatcggcgagcagccgtgggggcgggagttcgccctgcgcgacccggccggcaactgcg
tgcacttcgtggccgaggagcaggactgacacgtgctacgagatttcgattccaccgccgccttctatgaaaggttgggcttcggaatc
gttttccgggacgccggctggatgatcctccagcgcggggatctcatgctggagttcttcgcccaccccaacttgtttattgcagcttata
atggttacaaataaagcaatagcatcacaaatttcacaaataaagcatttttttcactgcattctagttgtggtttgtccaaactcatcaa
tgtatcttatcatgtctgtataccgtcgacctctagctagagcttggcgtaatcatggtcatagctgtttcctgtgtgaaattgttatccg
ctcacaattccacacaacatacgagccggaagcataaagtgtaaagcctggggtgcctaatgagtgagctaactcacattaattgcgttgc
gctcactgcccgctttccagtcgggaaacctgtcgtgccagctgcattaatgaatcggccaacgcgcggggagaggcggtttgcgtattgg
gcgctcttccgcttcctcgctcactgactcgctgcgctcggtcgttcggctgcggcgagcggtatcagctcactcaaaggcggtaatacgg
ttatccacagaatcaggggataacgcaggaaagaacatgtgagcaaaaggccagcaaaaggccaggaaccgtaaaaaggccgcgt
tgctggcgtttttccataggctccgcccccctgacgagcatcacaaaaatcgacgctcaagtcagaggtggcgaaacccgacaggac
tataaagataccaggcgtttccccctggaagctccctcgtgcgctctcctgttccgaccctgccgcttaccggatacctgtccgcctttct
cccttcgggaagcgtggcgctttctcatagctcacgctgtaggtatctcagttcggtgtaggtcgttcgctccaagctgggctgtgtgca
cgaaccccccgttcagcccgaccgctgcgccttatccggtaactatcgtcttgagtccaacccggtaagacacgacttatcgccactgg
cagcagccactggtaacaggattagcagagcgaggtatgtaggcggtgctacagagttcttgaagtggtggcctaactacggctaca
ctagaagaacagtatttggtatctgcgctctgctgaagccagttaccttcggaaaaagagttggtagctcttgatccggcaaacaaacca
ccgctggtagcggtggtttttttgtttgcaagcagcagattacgcgcagaaaaaaaggatctcaagaagatcctttgatcttttctacggg
gtctgacgctcagtggaacgaaaactcacgttaagggattttggtcatgagattatcaaaaaggatcttcacctagatccttttaaattaa
aaatgaagttttaaatcaatctaaagtatatatgagtaaacttggtctgacagttaccaatgcttaatcagtgaggcacctatctcagcga
tctgtctatttcgttcatccatagttgcctgactccccgtcgtgtagataactacgatacgggagggcttaccatctggccccagtgctgc
aatgataccgcgagacccacgctcaccggctccagatttatcagcaataaaccagccagccggaagggccgagcgcagaagtggtcct
gcaactttatccgcctccatccagtctattaattgttgccgggaagctagagtaagtagttcgccagttaatagtttgcgcaacgttgttg
ccattgctacaggcatcgtggtgtcacgctcgtcgtttggtatggcttcattcagctccggttcccaacgatcaaggcgagttacatgatcc
cccatgttgtgcaaaaaagcggttagctccttcggtcctccgatcgttgtcagaagtaagttggccgcagtgttatcactcatggttatgg
cagcactgcataattctcttactgtcatgccatccgtaagatgcttttctgtgactggtgagtactcaaccaagtcattctgagaatagtg
tatgcggcgaccgagttgctcttgcccggcgtcaatacgggataataccgcgccacatagcagaactttaaaagtgctcatcattggaaaa
cgttcttcggggcgaaaactctcaaggatcttaccgctgttgagatccagttcgatgtaacccactcgtgcacccaactgatcttcagcat
cttttactttcaccagcgtttctgggtgagcaaaaacaggaaggcaaaatgccgcaaaaaagggaataagggcgacacggaaatgttg
aatactcatactcttcctttttcaatattattgaagcatttatcagggttattgtctcatgagcggatacatatttgaatgtatttagaaa
aataaacaaataggggttccgcgcacatttccccgaaaagtgccacctgac
SEQ ID NO: 46 (Construct comprising a nucleic acid sequence encoding GFP, a m⁶A
reporter sequence, and DHFR; a nucleic acid sequenc encoding APOBEC1-YTH (5′-3′); and
a nucleic acid sequence encoding dsRed)
agggagtggtaaactcgactttcacttttctctatcactgatagggagtggtaaactcgactttcacttttctctatcactgatagggagt
ggtaaactcgactttcacttttctctatcactgatagggagtggtaaactcgactttcacttttctctatcactgatagggagtggtaaac
tcgagggggatccactagcatgaagggcgaattccagcacactggtaacccgtgtcggctccagatctggcctccgcgccgggttttggcg
cctcccgcgggcgcccccctcctcacggcgagccgcgttgacattgattattgactaggcttttgcaaaaagctttgcaaagatggataa
agttttaaacagagaggaatctttgcagctaatggaccttctaggtcttgaaaggagtgggaattggctccggtgcccgtcagtgggca
gagcgcacatcgcccacagtccccgagaagttggggggaggggtcggcaattgaaccggtgcctagagaaggtggcgcggggta
aactgggaaagtgatgtcgtgtactggctccgcctttttcccgagggtgggggagaaccgtatataagtgcagtagtcgccgtgaacgt
tctttttcgcaacgggtttgccgccagaacacaggtaagtgccgtgtgtggttcccgcgggcctggcctctttacgggttatggcccttg
cgtgccttgaattacttccacctggctgcagtacgtgattcttgatcccgagcttcgggttggaagtggggggagagttcgaggccttg
cgcttaaggagccccttcgcctcgtgcttgagttgaggcctggcctgggcgctggggccgccgcgtgcgaatctggtggcaccttcg
cgcctgtctcgctgctttcgataagtctctagccatttaaaatttttgatgacctgctgcgacgctttttttctggcaagatagtcttgta
aatgcgggccaagatctgcacactggtatttcggtttttggggccgcgggcggcgacggggcccgtgcgtcccagcgcacatgttcggcg
aggcggggcctgcgagcgcggccaccgagaatcggacgggggtagtctcaagctggccggcctgctctggtgcctggcctcgcg
ccgccgtgtatcgccccgccctgggcggcaaggctggcccggtcggcaccagttgcgtgagcggaaagatggccgcttcccggcc
ctgctgcagggagctcaaaatggaggacgcggcgctcgggagagcggggggtgagtcacccacacaaaggaaaagggcctttc
cgtcctcagccgtcgcttcatgtgactccacggagtaccgggcgccgtccaggcacctcgattagttctcgagcttttggagtacgtcgt
ctttaggttggggggaggggttttatgcgatggagtttccccacactgagtgggtggagactgaagttaggccagcttggcacttgatgt
aattctccttggaatttgccctttttgagtttggatcttggttcattctcaagcctcagacagtggttcaaagtttttttcttccatttca
ggtgtcgtgaggaattagcttggtactaatacgactcactatagggagacccaagctggctaggtaagcttggtaccgagctcggatccac
tagtccagtgtggtggaattctgcagatatccagcacagtggggtttagtgaaccgtcagatccgctagagatccgcggccgctaatacga
ctcactatagggagagccgccaccatgagctcagagactggcccagtggctgtggaccccacattgagacggcggatcgagccccat
gagtttgaggtattcttcgatccgagagagctccgcaaggagacctgcctgctttacgaaattaattgggggggccggcactccatttg
gcgacatacatcacagaacactaacaagcacgtcgaagtcaacttcatcgagaagttcacgacagaaagatatttctgtccgaacaca
aggtgcagcattacctggtttctcagctggagcccatgcggcgaatgtagtagggccatcactgaattcctgtcaaggtatccccacgt
cactctgtttatttacatcgcaaggctgtaccaccacgctgacccccgcaatcgacaaggcctgcgggatttgatctcttcaggtgtgact
atccaaattatgactgagcaggagtcaggatactgctggagaaactttgtgaattatagcccgagtaatgaagcccactggcctaggtat
ccccatctgtgggtacgactgtacgttcttgaactgtactgcatcatactgggcctgcctccttgtctcaacattctgagaaggaagcagc
cacagctgacattctttaccatcgctcttcagtcttgtcattaccagcgactgcccccacacattctctgggccaccgggttgaaaagcgg
cagcgagactcccgggacctcagagtccgccacaccagaaccccacccagtgttggagaagcttcggtccattaataactataaccc
caaagattttgactggaatctgaaacatggccgggttttcatcattaagagctactctgaggacgatattcaccgttccattaagtataat
atttggtgcagcacagagcatggtaacaagagactggatgctgcttatcgttccatgaacgggaaaggccccgtttacttacttttcagtg
tcaacggcagtggacacttctgtggcgtggcagaaatgaaatctgctgtggactacaacacatgtgcaggtgtgtggtcccaggacaaat
ggaagggtcgttttgatgtcaggtggatttttgtgaaggacgttcccaatagccaactgcgacacattcgcctagagaacaacgagaat
aaaccagtgaccaactctagggacactcaggaagtgcctctggaaaaggctaagcaggtgttgaaaattatagccagctacaagcac
accacttccatttttgatgacttctcacactatgagaaacgccaagaggaagaagaaagtgttaaaaaggaacgtcaaggtcgtgggaa
actcgagtacccctacgacgtgcccgactacgcctgagtttaaaatcgatggtacactcgaggttaacgaattctaccttacccagagtg
caggtgtgtggagatccctcctgccttgacattgagcagccttagagggtgggggaggctcaggggtcaggtctctgttcctgcttattg
gggagttcctggcctggcccttctatgtctccccaggtaccccagtttttctgggttcacccagagtgcagatgcttgaggaggtgggaa
gggactatttgggggtgtctggctcaggtgccatgcctcactggggctggttggcacctgcatttcctgggagtggggctgtctcaggg
tagctgggcacggtgttcccttgagtgggggtgtagtgagtgttcctagctgccacgcctttgccttcacctatgggatcgtggctgtca
gttaattaaccttccgcgggagctcacggggagagccccccgccaaagcccccagggatgtaattgcatccctcttccgctaggggg
cagcagcgagccgcccggggctccgctccggtccggcgctccccccgcatccccgagccggagccggcagcgtgcggggacag
cccggcacggggaaggtggcacgcgatcgctttcctctgaacgcttctcgctgctctttgagcctgcagacacctggggggatacgg
ggaaaaagctttaggctgaaagagagatttagaatgacagaatcatagaatggcctgggttgcaaaggagcacagtgctcacccagc
tccaaccccctgctatgtgcagggtcgccaaccagcagcccaggctgcccagagccacatccagcctggccttgaatgcctgcagg
gatggggcatccacagcctccttgggcaacctgttcagtgcgtcacggatccaattccacggggttggggttgcgccttttccaaggca
gccctgggtttgcgcagggacgcggctgctctgggcgtggttccgggaaacgcagcggcgccgaccctgggtctcgcacattcttca
cgtccgttcgcagcgtcacccggatcttcgccgctacccttgtgggccccccggcgacgcttcctgctccgcccctaagtcgggaag
gttccttgcggttcgcggcgtgccggacgtgacaaacggaagccgcacgtctcactagtaccctcgcagacggacagcgccaggga
gcaatggcagcgcgccgaccgcgatgggctgtggccaatagcggctgctcagcagggcgcgccgagagcagcggccgggaag
gggcggtgcgggaggcggggtgtggggcggtagtgtgggccctgttcctgcccgcgcggtgttccgcattctgcaagcctccggag
cgcacgtcggcagtcggctccctcgttgaccgaatcaccgacctctctccccagctgtagctagcacaaccatggatagcactgagaa
cgtcatcaagcccttcatgcgcttcaaggtgcacatggagggctccgtgaacggccacgagttcgagatcgagggcgagggcgagg
gcaagccctacgagggcacccagaccgccaagctgcaggtgaccaagggcggccccctgcccttcgcctgggacatcctgtcccc
ccagttccagtacggctccaaggtgtacgtgaagcaccccgccgacatccccgactacaagaagctgtccttccccgagggcttcaa
gtgggagcgcgtgatgaacttcgaggacggcggcgtggtgaccgtgacccaggactcctccctgcaggacggcaccttcatctacc
acgtgaagttcatcggcgtgaacttcccctccgacggccccgtaatgcagaagaagactctgggctgggagccctccaccgagcgc
ctgtacccccgcgacggcgtgctgaagggcgagatccacaaggcgctgaagctgaagggcggcggccactacctggtggagttca
agtcaatctacatggccaagaagcccgtgaagctgcccggctactactacgtggactccaagctggacatcacctcccacaacgagg
actacaccgtggtggagcagtacgagcgcgccgaggcccgccaccacctgttccagtagggctagctggtccggactgtactgggt
ctctctggttagaccagatctgagcctgggagctctctggctaactagggaacccactgcttaagcctcaataaagcttgccttgagtgc
ttcaagtagtgtgtgcccgtctgttgtgtgactctggtaactagagatccctcagacccttttagtcagtgtggaaaatctctagcagggc
ccgtttaaacccgctgatcagcctcgactgtgccttctagttgccagccatctgttgtttgcccctcccccgtgccttccttgaccctgga
aggtgccactcccactgtcctttcctaataaaatgaggaaattgcatcgcattgtctgagtaggtgtcattctattctggggggtgggggg
ggcaggacagcaagggggaggattgggaagacaatagcaggcatgctggggatgcggtgggctctatggcttctgaggcggaaag
aaccagctggggctctagggggtatccccacgcgccctgtagcggcgcattaagcgcggcgggtgtggtggttacgcgcagcgtga
ccgctacacttgccagcgccctagcgcccgctcctttcgctttcttcccttcctttctcgccacgttcgccggctttccccgtcaagctct
aaatcgggggctccctttagggttccgatttagtgctttacggcacctcgaccccaaaaaacttgattagggtgatggttcacgtagtggg
ccatcgccctgatagacggtttttcgccctttgacgttggagtccacgttctttaatagtggactcttgttccaaactggaacaacactca
accctatctcggtctattcttttgatttataagggattttgccgatttcggcctattggttaaaaaatgagctgatttaacaaaaatttaa
cgcgaattaattctgtggaatgtgtgtcagttagggtgtggaaagtccccaggctccccagcaggcagaagtatgcaaagcatgcatctca
attagtcagcaaccaggtgtggaaagtccccaggctccccagcaggcagaagtatgcaaagcatgcatctcaattagtcagcaaccatagt
cccgcccctaactccgcccatcccgcccctaactccgcccagttccgcccattctccgccccatggctgactaattttttttatttatgca
gaggccgaggccgcctctgcctctgagctattccagaagtagtgaggaggcttttttggaggcctaggcttttgcaaaaagctcccggga
gcttgtatatccattttcggatctgatcagcacgtgttgacaattaatcatcggcatagtatatcggcatagtataatacgacaaggtgag
gaactaaaccatggccaagttgaccagtgccgttccggtgctcaccgcgcgcgacgtcgccggagcggtcgagttctggaccgaccg
gctcgggttctcccgggacttcgtggaggacgacttcgccggtgtggtccgggacgacgtgaccctgttcatcagcgcggtccagga
ccaggtggtgccggacaacaccctggcctgggtgtgggtgcgcggcctggacgagctgtacgccgagtggtcggaggtcgtgtcc
acgaacttccgggacgcctccgggccggccatgaccgagatcggcgagcagccgtgggtgggggagttcgccctgcgcgaccc
ggccggcaactgcgtgcacttcgtggccgaggagcaggactgacacgtgctacgagatttcgattccaccgccgccttctatgaaag
gttgggcttcggaatcgttttccgggacgccggctggatgatcctccagcgcggggatctcatgctggagttcttcgcccaccccaact
tgtttattgcagcttataatggttacaaataaagcaatagcatcacaaatttcacaaataaagcatttttttcactgcattctagttgtgg
tttgtccaaactcatcaatgtatcttatcatgtctgtataccgtcgacctctagctagagcttggcgtaatcatggtcatagctgtttcct
gtgtgaaattgttatccgctcacaattccacacaacatacgagccggaagcataaagtgtaaagcctggggtgcctaatgagtgagctaac
tcacattaattgcgttgcgctcactgcccgctttccagtcgggaaacctgtcgtgccagctgcattaatgaatcggccaacgcgcggggag
aggcggtttgcgtattgggcgctcttccgcttcctcgctcactgactcgctgcgctcggtcgttcggctgcggcgagcggtatcagctcac
tcaaaggcggtaatacggttatccacagaatcaggggataacgcaggaaagaacatgtgagcaaaaggccagcaaaaggccaggaac
cgtaaaaaggccgcgttgctggcgtttttccataggctccgcccccctgacgagcatcacaaaaatcgacgctcaagtcagaggtggc
gaaacccgacaggactataaagataccaggcgtttccccctggaagctccctcgtgcgctctcctgttccgaccctgccgcttaccgg
atacctgtccgcctttctcccttcgggaagcgtggcgctttctcatagctcacgctgtaggtatctcagttcggtgtaggtcgttcgctcc
aagctgggctgtgtgcacgaaccccccgttcagcccgaccgctgcgccttatccggtaactatcgtcttgagtccaacccggtaagaca
cgacttatcgccactggcagcagccactggtaacaggattagcagagcgaggtatgtaggcggtgctacagagttcttgaagtggtgg
cctaactacggctacactagaagaacagtatttggtatctgcgctctgctgaagccagttaccttcggaaaaagagttggtagctcttgat
ccggcaaacaaaccaccgctggtagcggtggtttttttgtttgcaagcagcagattacgcgcagaaaaaaaggatctcaagaagatcct
ttgatcttttctacggggtctgacgctcagtggaacgaaaactcacgttaagggattttggtcatgagattatcaaaaaggatcttcacct
agatccttttaaattaaaaatgaagttttaaatcaatctaaagtatatatgagtaaacttggtctgacagttaccaatgcttaatcagtga
ggcacctatctcagcgatctgtctatttcgttcatccatagttgcctgactccccgtcgtgtagataactacgatacgggagggcttacca
tctggccccagtgctgcaatgataccgcgagacccacgctcaccggctccagatttatcagcaataaaccagccagccggaagggccgagc
gcagaagtggtcctgcaactttatccgcctccatccagtctattaattgttgccgggaagctagagtaagtagttcgccagttaatagttt
gcgcaacgttgttgccattgctacaggcatcgtggtgtcacgctcgtcgtttggtatggcttcattcagctccggttcccaacgatcaagg
cgagttacatgatcccccatgttgtgcaaaaaagcggttagctccttcggtcctccgatcgttgtcagaagtaagttggccgcagtgttat
cactcatggttatggcagcactgcataattctcttactgtcatgccatccgtaagatgcttttctgtgactggtgagtactcaaccaagtc
attctgagaatagtgtatgcggcgaccgagttgctcttgcccggcgtcaatacgggataataccgcgccacatagcagaactttaaaagtg
ctcatcattggaaaacgttcttcggggcgaaaactctcaaggatcttaccgctgttgagatccagttcgatgtaacccactcgtgcaccca
actgatcttcagcatcttttactttcaccagcgtttctgggtgagcaaaaacaggaaggcaaaatgccgcaaaaaagggaataagggcga
cacggaaatgttgaatactcatactcttcctttttcaatattattgaagcatttatcagggttattgtctcatgagcggatacatatttga
atgtatttagaaaaataaacaaataggggttccgcgcacatttccccgaaaagtgccacctgac
SEQ ID NO: 47 (Construct comprising a nucleic acid sequence encoding GFP-PEST, a m⁶A
reporter sequence, and DHFR; a nucleic acid sequenc encoding APOBEC1-YTH (5′-3′)
agggagtggtaaactcgactttcacttttctctatcactgatagggagtggtaaactcgactttcacttttctctatcactgatagggagt
ggtaaactcgactttcacttttctctatcactgatagggagtggtaaactcgactttcacttttctctatcactgatagggagtggtaaac
tcgagggggatccactagcatgaagggcgaattccagcacactggtaacccgtgtcggctccagatctggcctccgcgccgggttttggcg
cctcccgcgggcgcccccctcctcacggcgagccgcgttgacattgattattgactaggcttttgcaaaaagctttgcaaagatggataa
agttttaaacagagaggaatctttgcagctaatggaccttctaggtcttgaaaggagtgggaattggctccggtgcccgtcagtgggca
gagcgcacatcgcccacagtccccgagaagttggggggaggggtcggcaattgaaccggtgcctagagaaggtggcgcggggta
aactgggaaagtgatgtcgtgtactggctccgcctttttcccgaggggtggggagaaccgtatataagtgcagtagtcgccgtgaacgt
tctttttcgcaacgggtttgccgccagaacacaggtaagtgccgtgtgtggttcccgcgggcctggcctctttacgggttatggcccttg
cgtgccttgaattacttccacctggctgcagtacgtgattcttgatcccgagcttcgggttggaagtggggggagagttcgaggccttg
cgcttaaggagccccttcgcctcgtgcttgagttgaggcctggcctgggcgctggggccgccgcgtgcgaatctggtggcaccttcg
cgcctgtctcgctgctttcgataagtctctagccatttaaaatttttgatgacctgctgcgacgctttttttctggcaagatagtcttgta
aatgcgggccaagatctgcacactggtatttcggtttttggggccgcgggcggcgacggggcccgtgcgtcccagcgcacatgttcggcg
aggcggggcctgcgagcgcggccaccgagaatcggacgggggtagtctcaagctggccggcctgctctggtgcctggcctcgcg
ccgccgtgtatcgccccgccctgggcggcaaggctggcccggtcggcaccagttgcgtgagcggaaagatggccgcttcccggcc
ctgctgcagggagctcaaaatggaggacgcggcgctcgggagagcggggggtgagtcacccacacaaaggaaaagggcctttc
cgtcctcagccgtcgcttcatgtgactccacggagtaccgggcgccgtccaggcacctcgattagttctcgagcttttggagtacgtcgt
ctttaggttggggggaggggttttatgcgatggagtttccccacactgagtgggtggagactgaagttaggccagcttggcacttgatgt
aattctccttggaatttgccctttttgagtttggatcttggttcattctcaagcctcagacagtggttcaaagtttttttcttccatttca
ggtgtcgtgaggaattagcttggtactaatacgactcactatagggagacccaagctggctaggtaagcttggtaccgagctcggatccac
tagtccagtgtggtggaattctgcagatatccagcacagtggggtttagtgaaccgtcagatccgctagagatccgcggccgctaatacga
ctcactatagggagagccgccaccatgagctcagagactggcccagtggctgtggaccccacattgagacggcggatcgagccccat
gagtttgaggtattcttcgatccgagagagctccgcaaggagacctgcctgctttacgaaattaattgggggggccggcactccatttg
gcgacatacatcacagaacactaacaagcacgtcgaagtcaacttcatcgagaagttcacgacagaaagatatttctgtccgaacaca
aggtgcagcattacctggtttctcagctggagcccatgcggcgaatgtagtagggccatcactgaattcctgtcaaggtatccccacgt
cactctgtttatttacatcgcaaggctgtaccaccacgctgacccccgcaatcgacaaggcctgcgggatttgatctcttcaggtgtgact
atccaaattatgactgagcaggagtcaggatactgctggagaaactttgtgaattatagcccgagtaatgaagcccactggcctaggtat
ccccatctgtgggtacgactgtacgttcttgaactgtactgcatcatactgggcctgcctccttgtctcaacattctgagaaggaagcagc
cacagctgacattctttaccatcgctcttcagtcttgtcattaccagcgactgcccccacacattctctgggccaccgggttgaaaagcgg
cagcgagactcccgggacctcagagtccgccacaccagaaccccacccagtgttggagaagcttcggtccattaataactataaccc
caaagattttgactggaatctgaaacatggccgggttttcatcattaagagctactctgaggacgatattcaccgttccattaagtataata
tttggtgcagcacagagcatggtaacaagagactggatgctgcttatcgttccatgaacgggaaaggccccgtttacttacttttcagtgtc
aacggcagtggacacttctgtggcgtggcagaaatgaaatctgctgtggactacaacacatgtgcaggtgtgtggtcccaggacaaat
ggaagggtcgttttgatgtcaggtggatttttgtgaaggacgttcccaatagccaactgcgacacattcgcctagagaacaacgagaat
aaaccagtgaccaactctagggacactcaggaagtgcctctggaaaaggctaagcaggtgttgaaaattatagccagctacaagcac
accacttccatttttgatgacttctcacactatgagaaacgccaagaggaagaagaaagtgttaaaaaggaacgtcaaggtcgtgggaa
actcgagtacccctacgacgtgcccgactacgcctgagtttaaaatcgatggtacactcgaggttaacgaattctaccttacccagagtg
caggtgtgtggagatccctcctgccttgacattgagcagccttagaggggggggaggctcaggggtcaggtctctgttcctgcttattg
gggagttcctggcctggcccttctatgtctccccaggtaccccagtttttctgggttcacccagagtgcagatgcttgaggaggtgggaa
gggactatttgggggtgtctggctcaggtgccatgcctcactggggctggttggcacctgcatttcctgggagtggggctgtctcaggg
tagctgggcacggtgttcccttgagtgggggtgtagtgagtgttcctagctgccacgcctttgccttcacctatgggatcgtggctgtca
gttaattaaccttccgcgggagctcacggggagagccccccgccaaagcccccagggatgtaattgcatccctcttccgctaggggg
cagcagcgagccgcccggggctccgctccggtccggcgctccccccgcatccccgagccggagccggcagcgtgcggggacag
cccggcacggggaaggtggcacgcgatcgctttcctctgaacgcttctcgctgctctttgagcctgcagacacctggggggatacgg
ggaaaaagctttaggctgaaagagagatttagaatgacagaatcatagaatggcctgggttgcaaaggagcacagtgctcacccagc
tccaaccccctgctatgtgcagggtcgccaaccagcagcccaggctgcccagagccacatccagcctggccttgaatgcctgcagg
gatggggcatccacagcctccttgggcaacctgttcagtgcgtcacggatccaattccacggggttggggttgcgccttttccaaggca
gccctgggtttgcgcagggacgcggctgctctgggcgtggttccgggaaacgcagcggcgccgaccctgggtctcgcacattcttca
cgtccgttcgcagcgtcacccggatcttcgccgctacccttgtgggccccccggcgacgcttcctgctccgcccctaagtcgggaag
gttccttgcggttcgcggcgtgccggacgtgacaaacggaagccgcacgtctcactagtaccctcgcagacggacagcgccaggga
gcaatggcagcgcgccgaccgcgatgggctgtggccaatagcggctgctcagcagggcgcgccgagagcagcggccgggaag
gggcggtgcgggaggcggggtgtggggcggtagtgtgggccctgttcctgcccgcgcggtgttccgcattctgcaagcctccggag
cgcacgtcggcagtcggctccctcgttgaccgaatcaccgacctctctccccagctgtagctagcacaaccatggatagcactgagaa
cgtcatcaagcccttcatgcgcttcaaggtgcacatggagggctccgtgaacggccacgagttcgagatcgagggcgagggcgagg
gcaagccctacgagggcacccagaccgccaagctgcaggtgaccaagggcggccccctgcccttcgcctgggacatcctgtcccc
ccagttccagtacggctccaaggtgtacgtgaagcaccccgccgacatccccgactacaagaagctgtccttccccgagggcttcaa
gtgggagcgcgtgatgaacttcgaggacggcggcgtggtgaccgtgacccaggactcctccctgcaggacggcaccttcatctacc
acgtgaagttcatcggcgtgaacttcccctccgacggccccgtaatgcagaagaagactctgggctgggagccctccaccgagcgc
ctgtacccccgcgacggcgtgctgaagggcgagatccacaaggcgctgaagctgaagggcggcggccactacctggtggagttca
agtcaatctacatggccaagaagcccgtgaagctgcccggctactactacgtggactccaagctggacatcacctcccacaacgagg
actacaccgtggtggagcagtacgagcgcgccgaggcccgccaccacctgttccagtagggctagctggtccggactgtactgggt
ctctctggttagaccagatctgagcctgggagctctctggctaactagggaacccactgcttaagcctcaataaagcttgccttgagtgc
ttcaagtagtgtgtgcccgtctgttgtgtgactctggtaactagagatccctcagacccttttagtcagtgtggaaaatctctagcagggcc
cgtttaaacccgctgatcagcctcgactgtgccttctagttgccagccatctgttgtttgcccctcccccgtgccttccttgaccctggaag
gtgccactcccactgtcctttcctaataaaatgaggaaattgcatcgcattgtctgagtaggtgtcattctattctggggggtggggggg
gcaggacagcaagggggaggattgggaagacaatagcaggcatgctggggatgcggtgggctctatggcttctgaggcggaaag
aaccagctggggctctagggggtatccccacgcgccctgtagcggcgcattaagcgcggcgggtgtggtggttacgcgcagcgtga
ccgctacacttgccagcgccctagcgcccgctcctttcgctttcttcccttcctttctcgccacgttcgccggctttccccgtcaagctcta
aatcgggggctccctttagggttccgatttagtgctttacggcacctcgaccccaaaaaacttgattagggtgatggttcacgtagtggg
ccatcgccctgatagacggtttttcgccctttgacgttggagtccacgttctttaatagtggactcttgttccaaactggaacaacactcaa
ccctatctcggtctattcttttgatttataagggattttgccgatttcggcctattggttaaaaaatgagctgatttaacaaaaatttaacg
cgaattaattctgtggaatgtgtgtcagttagggtgtggaaagtccccaggctccccagcaggcagaagtatgcaaagcatgcatctcaatt
agtcagcaaccaggtgtggaaagtccccaggctccccagcaggcagaagtatgcaaagcatgcatctcaattagtcagcaaccatagt
cccgcccctaactccgcccatcccgcccctaactccgcccagttccgcccattctccgccccatggctgactaattttttttatttatgcag
aggccgaggccgcctctgcctctgagctattccagaagtagtgaggaggcttttttggaggcctaggcttttgcaaaaagctcccggga
gcttgtatatccattttcggatctgatcagcacgtgttgacaattaatcatcggcatagtatatcggcatagtataatacgacaaggtgagg
aactaaaccatggccaagttgaccagtgccgttccggtgctcaccgcgcgcgacgtcgccggagcggtcgagttctggaccgaccg
gctcgggttctcccgggacttcgtggaggacgacttcgccggtgtggtccgggacgacgtgaccctgttcatcagcgcggtccagga
ccaggtggtgccggacaacaccctggcctgggtgtgggtgcgcggcctggacgagctgtacgccgagtggtcggaggtcgtgtcc
acgaacttccgggacgcctccgggccggccatgaccgagatcggcgagcagccgtggggggggagttcgccctgcgcgaccc
ggccggcaactgcgtgcacttcgtggccgaggagcaggactgacacgtgctacgagatttcgattccaccgccgccttctatgaaag
gttgggcttcggaatcgttttccgggacgccggctggatgatcctccagcgcggggatctcatgctggagttcttcgcccaccccaact
tgtttattgcagcttataatggttacaaataaagcaatagcatcacaaatttcacaaataaagcatttttttcactgcattctagttgtggt
ttgtccaaactcatcaatgtatcttatcatgtctgtataccgtcgacctctagctagagcttggcgtaatcatggtcatagctgtttcctgt
gtgaaattgttatccgctcacaattccacacaacatacgagccggaagcataaagtgtaaagcctggggtgcctaatgagtgagctaactca
cattaattgcgttgcgctcactgcccgctttccagtcgggaaacctgtcgtgccagctgcattaatgaatcggccaacgcgcggggagaggc
ggtttgcgtattgggcgctcttccgcttcctcgctcactgactcgctgcgctcggtcgttcggctgcggcgagcggtatcagctcactca
aaggcggtaatacggttatccacagaatcaggggataacgcaggaaagaacatgtgagcaaaaggccagcaaaaggccaggaac
cgtaaaaaggccgcgttgctggcgtttttccataggctccgcccccctgacgagcatcacaaaaatcgacgctcaagtcagaggtggc
gaaacccgacaggactataaagataccaggcgtttccccctggaagctccctcgtgcgctctcctgttccgaccctgccgcttaccgg
atacctgtccgcctttctcccttcgggaagcgtggcgctttctcatagctcacgctgtaggtatctcagttcggtgtaggtcgttcgctcca
agctgggctgtgtgcacgaaccccccgttcagcccgaccgctgcgccttatccggtaactatcgtcttgagtccaacccggtaagaca
cgacttatcgccactggcagcagccactggtaacaggattagcagagcgaggtatgtaggcggtgctacagagttcttgaagtggtgg
cctaactacggctacactagaagaacagtatttggtatctgcgctctgctgaagccagttaccttcggaaaaagagttggtagctcttgat
ccggcaaacaaaccaccgctggtagcggtggtttttttgtttgcaagcagcagattacgcgcagaaaaaaaggatctcaagaagatcct
ttgatcttttctacggggtctgacgctcagtggaacgaaaactcacgttaagggattttggtcatgagattatcaaaaaggatcttcaccta
gatccttttaaattaaaaatgaagttttaaatcaatctaaagtatatatgagtaaacttggtctgacagttaccaatgcttaatcagtgagg
cacctatctcagcgatctgtctatttcgttcatccatagttgcctgactccccgtcgtgtagataactacgatacgggagggcttaccatct
ggccccagtgctgcaatgataccgcgagacccacgctcaccggctccagatttatcagcaataaaccagccagccggaagggccgagc
gcagaagtggtcctgcaactttatccgcctccatccagtctattaattgttgccgggaagctagagtaagtagttcgccagttaatagtttg
cgcaacgttgttgccattgctacaggcatcgtggtgtcacgctcgtcgtttggtatggcttcattcagctccggttcccaacgatcaaggc
gagttacatgatcccccatgttgtgcaaaaaagcggttagctccttcggtcctccgatcgttgtcagaagtaagttggccgcagtgttatc
actcatggttatggcagcactgcataattctcttactgtcatgccatccgtaagatgcttttctgtgactggtgagtactcaaccaagtcat
tctgagaatagtgtatgcggcgaccgagttgctcttgcccggcgtcaatacgggataataccgcgccacatagcagaactttaaaagtgct
catcattggaaaacgttcttcggggcgaaaactctcaaggatcttaccgctgttgagatccagttcgatgtaacccactcgtgcacccaa
ctgatcttcagcatcttttactttcaccagcgtttctgggtgagcaaaaacaggaaggcaaaatgccgcaaaaaagggaataagggcga
cacggaaatgttgaatactcatactcttcctttttcaatattattgaagcatttatcagggttattgtctcatgagcggatacatatttgaa
tgtatttagaaaaataaacaaataggggttccgcgcacatttccccgaaaagtgccacctgac
SEQ ID NO: 48 ([0001]
Atggtgagcaagggcgaggagctgttcaccggggtggtgcccatcctggtcgagctggatggcgatgtaaatggccacaagttcag
cgtgtccggcgagggcgagggcgatgccacctacggcaagctcaccctgaagttcatctgcaccaccggcaagctgcccgtgccct
ggcccaccctcgtcaccaccctcacctacggcgtgcagtgcttcagccgctaccccgatcacatgaagcagcacgatttcttcaagtc
cgccatgcccgaaggctacgtccaggagcgcaccatcttcttcaaggatgatggcaattaccgtacccgcgccgaggtgaagttcga
gggcgataccctggtgaatcgcatcgagctgaagggcatcgatttcaaggaggatggcaatatcctggggcacaagctggagtacaa
ttacaatagccacaatgtctatatcatggccgataagcagaagaatggcatcaaggtgaatttcaagatccgccacaatatcgaggatg
gcagcgtgcagctcgccgatcactaccagcagaatacccccatcggcgatggccccgtgctgctgcccgataatcactacctgagca
cccagtccgccctgagcaaagatcccaatgagaagcgcgatcacatggtcctgctggagttcgtcaccgccgccgggatcactctcg
gcatggatgagctgtacaagttgcttagccatggcttcccgccggaggtggaggagcaggatgatggcacgctgcccatgtcttgtgc
ccaggagagcgggatggaccgtcaccctgcagcctgtgcttctgctaggatcaatgtgttagatgcg
SEQ ID NO: 49 (Amino acid sequence of a GFP-PEST polypeptide)
MVSKGEELFTGVVPILVELDGDVNGHKFSVSGEGEGDATYGKLTLKFICTTGKLPVP
WPTLVTTLTYGVQCFSRYPDHMKQHDFFKSAMPEGYVQERTIFFKDDGNYRTRAEV
KFEGDTLVNRIELKGIDFKEDGNILGHKLEYNYNSHNVYIMADKQKNGIKVNFKIRH
NIEDGSVQLADHYQQNTPIGDGPVLLPDNHYLSTQSALSKDPNEKRDHMVLLEFVTA
AGITLGMDELYKLLSHGFPPEVEEQDDGTLPMSCAQESGMDRHPAACASARINVLD
A
SEQ ID NO: 50 (Exemplary m6A sensor sequence-5′-3′)
GCGGACTTACGACAGTTGCGTTACACCCTTTCTCGACAAAACCTAACTTGCGCAG
AAAACATGCCAATCTCATCTTGGCTT
SEQ ID NO: 51 (Exemplary m6A sensor sequence-5′-3′)
GCGGCGTTACGACAGTTGCGTTACACCCTTTCTCGACAAAACCTAACTTGCGCAG
AAAACATGCCAATCTCATCTTGGCTT
SEQ ID NO: 52 (Exemplary m6A sensor sequence-5′-3′)
GCGGACTTACGTCAGTTGCGTTACACCCTTTCTCGACAAAACCTAACTTGCGCAG
AAAACATGCCAATCTCATCTTGGCTT
SEQ ID NO: 53 (Exemplary m6A sensor sequence-5′-3′)
GCGGAGTTACGACAGTTGCGTTACACCCTTTCTCGTCAAAACCTAACTTGCGCAG
AAAACATGCCAATCTCATCTTGGCTT
SEQ ID NO: 54 (Exemplary m6A sensor sequence-5′-3′)
GCGGAGTTACGACAGTTGCGTTACACCCTTTCTCGACAAAGCCTAACTTGCGCAG
AAAACATGCCAATCTCATCTTGGCTT
SEQ ID NO: 55 (Exemplary m6A sensor sequence-5′-3′)
GCGGAGTTACGACAGTTGCGTTACACCCTTTCTCGACAAAACCTAGCTTGCGCAG
AAAACATGCCAATCTCATCTTGGCTT
SEQ ID NO: 56 (Exemplary m6A sensor sequence-5′-3′)
GCGGAGTTACGACAGTTGCGTTACACCCTTTCTCGACAAAACCTAACTTGCGCAG
AAAGCATGCCAATCTCATCTTGGCTT
SEQ ID NO: 57 (Exemplary m6A sensor sequence-5′-3′)
GCGGACTTACGACAGTTGCGTCCAATCTCATCTTGGCTT
SEQ ID NO: 58 (Exemplary m6A sensor sequence-5′-3′)
GCGGCCTTACGTCAGTTGCGTTACACCCTTTCTCGGCAAAGCCTAGCTTGCGCAG
AAAGCATGCCAATCTCATCTTGGCTT
SEQ ID NO: 59 ((GGGGS)₂₀)
SEQ ID NO: 60-deaminase domain of rAPOBEC1
RRRIEPHEFEVFFDPRELRKETCLLYEINWGGRHSIWRHTSQNTNKHVEVNFIEKFTT
ERYFCPNTRCSITWFLSWSPCGECSRAITEFLSRYPHVTLFIYIARLYHHADPRNRQGL
RDLISSGVTIQIMTEQESGYCWRNFVNYSPSNEAHWPRYPHLWVRLYVLELYCIILGL
PPCLNILRRKQPQLTFFTIALQSCHYQRLPPHILWATGLK
SEQ ID NO: 61-deaminase domain of hAICDA
LMNRRKFLYQFKNVRWAKGRRETYLCYVVKRRDSATSFSLDFGYLRNKNGCHVEL
LFLRYISDWDLDPGRCYRVTWFTSWSPCYDCARHVADFLRGNPNLSLRIFTARLYFC
EDRKAEPEGLRRLHRAGVQIAIMTFKDYFYCWNTFVENHERTFKAWEGLHENSVRL
SRQLRRILLPL
SEQ ID NO: 62-deaminase domain of hAPOBEC3A
TSNFNNGIGRHKTYLCYEVERLDNGTSVKMDQHRGFLHNQAKNLLCGFYGRHAEL
RFLDLVPSLQLDPAQIYRVTWFISWSPCFSWGCAGEVRAFLQENTHVRLRIFAARIYD
YDPLYKEALQMLRDAGAQVSIMTYDEFKHCWDTFVDHQGCPFQPWDGLDEHSQAL
SGRLR
SEQ ID NO: 63-catalytic domain of ADAR2
MDSLLMNRREFLYQFKNVRWAKGRRETYLCYVVKRRDSATSFSLDFGYLRNKNGC
HVELLFLRYISDWDLDPGRCYRVTWFISWSPCYDCARHVADFLRGNPNLSLRIFTAR
LYFCEAGRREPEGLRRLHRAGVQIAIMTFKDYFYCWNTFVENHGRTFKAWEGLHEN
SVRLSRQLRRILL

What is claimed is:

1. An expression system comprising:
(a) a first DNA construct comprising a nucleic acid sequence encoding a fusion protein, wherein the fusion protein comprises an N⁶-methyladenosine (m⁶A) binding domain of a YT521-B homology (YTH) domain-containing protein fused to a catalytic domain of a cytidine deaminase or a catalytic domain of an adenosine deaminase; and
(b) a second DNA construct comprising:
i. a nucleic acid sequence encoding a heterologous polypeptide;
ii. a m⁶A sensor sequence; and
iii. a polypeptide encoding dihydrofolate reductase (DHFR).

2. The expression system of claim 1, wherein the m⁶A sensor sequence comprises SEQ ID NO: 16 (GACTTACGACAG).

3. The expression system of claim 1, wherein the m⁶A binding domain is fused to the catalytic domain via a peptide linker.

4. The expression system of claim 1, wherein the m⁶A binding domain comprises a polypeptide having at least 95% identity to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

5. The expression system of claim 1, wherein the catalytic domain comprises a polypeptide having at least 95% identity to SEQ ID NO 12 or a catalytic fragment thereof, SEQ ID NO: 13 or a catalytic fragment thereof; SEQ ID NO: 14 or a catalytic fragment thereof; or SEQ ID NO: 15.

6. The expression system of claim 1, wherein a vector comprises the first DNA construct.

7. The expression system of claim 1, wherein a vector comprises the second DNA construct.

8. The expression system of claim 1, wherein a vector comprises the first DNA construct and the second DNA construct.

9. The expression system of claim 1, wherein the nucleic acid sequence encoding a fusion protein and/or the nucleic acid sequence encoding a heterologous polypeptide and a polypeptide encoding dihydrofolate reductase (DHFR) are operably linked to a first promoter.

10. The expression system of claim 1, wherein the vector further comprises a nucleic acid sequence encoding a selectable marker operably linked to a second promoter.

11. The expression system of claim 10, wherein the selectable marker is a fluorescent protein.

12. The expression system of claim 11, wherein the fluorescent protein is dsRed.

13. The expression system of claim 9, wherein the promoter is a constitutive or an inducible promoter.

14. The expression system of claim 1, wherein the cytidine deaminase is APOBEC-1.

15. The expression system of claim 1, wherein the heterologous polypeptide is a reporter protein.

16. The expression system of claim 15, wherein the reporter protein is a fluorescent protein.

17. The expression system of claim 16, wherein the fluorescent protein is a green fluorescent protein.

18. A nucleic acid sequence comprising a nucleic acid sequence encoding a heterologous polypeptide, a m⁶A sensor sequence, and, a polypeptide encoding dihydrofolate reductase (DHFR).

19. A vector comprising the expression system of claim 1.

20. A host cell comprising the expression system of claim 1 or the vector of claim 19.

21. The host cell of claim 20, wherein the cell expresses a reporter protein that is not encoded by the first DNA construct or the second DNA construct.

22. A non-human transgenic animal comprising the host cell of claim 20.

23. A method for detecting m⁶A methylation-dependent expression of a heterologous polypeptide in one or more cells comprising:
(a) introducing the expression system of claim 1 into one or more cells;
(b) detecting expression of the heterologous protein, wherein expression of the heterologous protein is indicative of m⁶A methylation-dependent expression of the heterologous polypeptide in the one or more cells.

24. A method for detecting m⁶A methylation in one or more cells comprising
(a) introducing the expression system of claim 15 into one or more cells;
(b) detecting expression of the reporter protein.

25. A method for detecting in vitro m⁶A methylation in one or more cells comprising
(a) contacting one or more cells with
i. a fusion protein comprising an N⁶-methyladenosine (m⁶A) binding domain of a YT521-B homology (YTH) domain-containing protein fused to a catalytic domain of a cytidine deaminase or a catalytic domain of an adenosine deaminase; and
ii. a DNA construct comprising a nucleic acid sequence encoding a heterologous polypeptide, a m⁶A sensor sequence, and a polypeptide encoding dihydrofolate reductase (DHFR); and
(b) detecting expression of the reporter protein.

26. A method for identifying an agent that modulates m⁶A methylation in a cell comprising:
(a) contacting one or more cells comprising the reporter protein expression systems of claim 15 with an agent; and
(b) detecting expression of the reporter protein, wherein a decrease in expression of the reporter protein as compared to expression of the reporter protein in the absence of the agent indicates the agent decreases m⁶A methylation in the one or more cells, and wherein an increase in expression of the reporter protein as compared to expression of the reporter protein in the absence of the agent indicates the agent increases m⁶A methylation in the cell.

27. A method for identifying an agent that inhibits METTL3-dependent methylation in a cell comprising:
(a) contacting one or more cells comprising the expression system of claim 15 with an agent;
(b) detecting expression of the reporter protein, wherein a decrease in expression of the reporter protein as compared to expression of the reporter protein in the absence of the agent indicates the agent inhibits METTL3-dependent methylation in the one or more cells.

28. The method of claim 26, wherein the agent is a small molecule, a polypeptide or a nucleic acid.

29. The method of claim 23, wherein the one or more cells are in vitro cells.

30. The method of claim 23, wherein the cell is a cell from a subject.

31. The method of claim 23 wherein the cell is in a subject.

32. A method for identifying an agent that modulates m⁶A methylation in a non-human transgenic animal comprising:
(a) contacting a non-human transgenic animal that comprises the expression system of claim 15 with an agent;
(b) detecting expression of the reporter protein, wherein a decrease in expression of the reporter protein as compared to expression of the reporter protein in the absence of the agent indicates the agent decreases m⁶A methylation, and wherein an increase in expression of the reporter protein as compared to expression of the reporter protein in the absence of the agent indicates the agent increases m⁶A methylation.

33. A method for identifying an agent that inhibits METTL3-dependent methylation in a non-human transgenic animal comprising:
(a) contacting a non-human transgenic animal that comprises the expression system of claim 15 with an agent;
(b) detecting expression of the reporter protein, wherein a decrease in expression of the reporter protein as compared to expression of the reporter protein in the absence of the agent indicates the agent inhibits METTL3-dependent methylation.

34. The method of claim 32, wherein the expression system is expressed in a cell-specific or tissue-specific manner in the non-human transgenic animal.

35. The method of claim 23, further comprising isolating RNA from the one or more cells, amplifying one or more target sequences in the RNA, and identifying cytidine to uridine deamination at sites adjacent to one or more m⁶A residues in the one or more target sequences.

36. The method of claim 34, wherein the one or more target sequences are amplified by reverse transcriptase polymerase chain reaction (RT-PCR).

37. The method of claim 34, wherein cytidine to uridine deamination is identified by sequencing the one or more target sequences.

38. The method of claim 32, further comprising isolating RNA from one or more cells of the non-human transgenic animal, amplifying one or more target sequences in the RNA, and identifying cytidine to uridine deamination at sites adjacent to one or more mA residues in the one or more target sequences.

39. The method of claim 38, wherein the one or more target sequences are amplified by reverse transcriptase polymerase chain reaction (RT-PCR).

40. The method of claim 38, wherein cytidine to uridine deamination is identified by sequencing the one or more target sequences.

41. A kit comprising the expression system of claim 1.


SEQUENCE LISTING (63 sequences)

SEQ ID NO: 1 - protein, 195 aa, synthetic construct
PHPVLEKLRSINNYNPKDFDWNLKHGRVFIIKSYSEDDIHRSIKYNIWCSTEHGNKRLDAAYRSMNGKGPVYLLFSVNGSGHFCGVAEMKSAVDYNTCAGVWSQDKWKGRFDVRWIFVKDVPNSQLRHIRLENNENKPVTNSRDTQEVPLEKAKQVLKIIASYKHTTSIFDDFSHYEKRQEEEESVKKERQGRGK

SEQ ID NO: 2 - protein, 195 aa, synthetic construct
PHPVLEKLRSINNYNPKDFDWNLKHGRVFIIKSYSEDDIHRSIKYNIACSTEHGNKRLDAAYRSMNGKGPVYLLFSVNGSGHFCGVAEMKSAVDYNTCAGVASQDKWKGRFDVRWIFVKDVPNSQLRHIRLENNENKPVTNSRDTQEVPLEKAKQVLKIIASYKHTTSIFDDFSHYEKRQEEEESVKKERQGRGK

SEQ ID NO: 3 - protein, 170 aa, synthetic construct
GRVFIIKSYSEDDIHRSIKYNIWCSTEHGNKRLDAAYRSMNGKGPVYLLFSVNGSGHFCGVAEMKSAVDYNTCAGVWSQDKWKGRFDVRWIFVKDVPNSQLRHIRLENNENKPVTNSRDTQEVPLEKAKQVLKIIASYKHTTSIFDDFSHYEKRQEEEESVKKERQGRGK

SEQ ID NO: 4 - protein, 170 aa, synthetic construct
GRVFIIKSYSEDDIHRSIKYNIACSTEHGNKRLDAAYRSMNGKGPVYLLFSVNGSGHFCGVAEMKSAVDYNTCAGVASQDKWKGRFDVRWIFVKDVPNSQLRHIRLENNENKPVTNSRDTQEVPLEKAKQVLKIIASYKHTTSIFDDFSHYEKRQEEEESVKKERQGRGK

SEQ ID NO: 5 - protein, 195 aa, synthetic construct
PHPVLEKLRSINNYNPKDFDWNLKHGRVFIIKSYSENDIHRSIKYNIWCSTEHGNKRLDAAYRSMNGKGPVYLLFSVNGSGHFCGVAEMKSAVDYNTCAGVWSQDKWKGRFDVRWIFVKDVPNSQLRHIRLENNENKPVTNSRDTQEVPLEKAKQVLKIIASYKHTTSIFDDFSHYEKRQEEEESVKKERQGRGK

SEQ ID NO: 6 - protein, 195 aa, synthetic construct
HPVLEKLKAAHSYNPKEFEWNLKSGRVFIIKSYSEDDIHRSIKYSIWCSTEHGNKRLDSAFRCMSSKGPVYLLFSVNGSGHFCGVAEMKSPVDYGTSAGVWSQDKWKGKFDVQWIFVKDVPNNQLRHIRLENNDNKPVTNSRDTQEVPLEKAKQVLKIISSYKHTTSIFDDFAHYEKRQEEEEVVRKERQSRNKQ

SEQ ID NO: 7 - protein, 171 aa, synthetic construct
GRVFIIKSYSEDDIHRSIKYSIWCSTEHGNKRLDSAFRCMSSKGPVYLLFSVNGSGHFCGVAEMKSPVDYGTSAGVWSQDKWKGKFDVQWIFVKDVPNNQLRHIRLENNDNKPVTNSRDTQEVPLEKAKQVLKIISSYKHTTSIFDDFAHYEKRQEEEEVVRKERQSRNKQ

SEQ ID NO: 8 - protein, 195 aa, synthetic construct
HPVLEKLKAAHSYNPKEFEWNLKSGRVFIIKSYSEDNIHRSIKYSIWCSTEHGNKRLDSAFRCMSSKGPVYLLFSVNGSGHFCGVAEMKSPVDYGTSAGVWSQDKWKGKFDVQWIFVKDVPNNQLRHIRLENNDNKPVTNSRDTQEVPLEKAKQVLKIISSYKHTTSIFDDFAHYEKRQEEEEVVRKERQSRNKQ

SEQ ID NO: 9 - protein, 195 aa, synthetic construct
VHPVLEKLKAINNYNPKDFDWNLKNGRVFIIKSYSEDDIHRSIKYSIWCSTEHGNKRLDAAYRSLNGKGPLYLLFSVNGSGHFCGVAEMKSVVDYNAYAGVWSQDKWKGKFEVKWIFVKDVPNNQLRHIRLENNDNKPVTNSRDTQEVPLEKAKQVLKIIATFKHTTSIFDDFAHYEKRQEEEEAMRRERNRNKQ

SEQ ID NO: 10 - protein, 165 aa, synthetic construct
SKLKYVLQDARFFLIKSNNHENVSLAKAKGVWSTLPVNEKKLNLAFRSARSVILIFSVRESGKFQGFARLSSESHHGGSPIHWVLPAGMSAKMLGGVFKIDWICRRELPFTKSAHLTNPWNEHKPVKIGRDGQEIELECGTQLCLLFPPDESIDLYQVIHKMRHK

SEQ ID NO: 11 - protein, 144 aa, synthetic construct
PVRYFIMKSSNLRNLEISQQKGIWSTTPSNERKLNRAFWESSIVYLVFSVQGSGHFQGFSRMSSEIGREKSQDWGSAGLGGVFKVEWIRKESLPFQFAHHLLNPWNDNKKVQISRDGQELEPLVGEQLLQLWERLPLGEKNTTD

SEQ ID NO: 12 - protein, 229 aa, synthetic construct [sequence omitted]

SEQ ID NO: 13 - protein, 198 aa, synthetic construct [sequence omitted]

SEQ ID NO: 14 - protein, 199 aa, synthetic construct
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 15 - protein, 385 aa, synthetic construct
QLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGQGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 16 - DNA, 12 bp, synthetic construct
  source 1..12: mol_type = other DNA
gacttacgacag

SEQ ID NO: 17 - protein, 181 aa, synthetic construct
MDSLLMNRREFLYQFKNVRWAKGRRETYLCYVVKRRDSATSFSLDFGYLRNKNGCHVELLFLRYISDWDLDPGRCYRVTWFISWSPCYDCARHVADFLRGNPNLSLRIFTARLYFCEAGRREPEGLRRLHRAGVQIAIMTFKDYFYCWNTFVENHGRTFKAWEGLHENSVRLSRQLRRILL

SEQ ID NO: 18 - protein, 15 aa, synthetic construct
SGSETPGTSESATPE

SEQ ID NO: 19 - protein, 16 aa, synthetic construct
SGSETPGTSESATPES

SEQ ID NO: 20 - protein, 15 aa, synthetic construct
GGGGSGGGGSGGGGS

SEQ ID NO: 21 - protein, 100 aa, synthetic construct
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 22 - protein, 17 aa, synthetic construct
AEAAAKEAAAKEAAAKA

SEQ ID NO: 23 - protein, 52 aa, synthetic construct
AEAAAKEAAAKEAAAKEAAAKEAAAKEAAAKEAAAKEAAAKEAAAKEAAAKA

SEQ ID NO: 24 - protein, 102 aa, synthetic construct
AEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 25 - protein, 1368 aa, synthetic construct
ATGAGCAGCGAAACCGGTCCGGTGGCGGTTGACCCGACCCTGCGTCGTCGTATTGAGCCGCACGAGTTCGAAGTGTTCTTTGATCCGCGTGAGCTGCGTAAGGAAACCTGCCTGCTGTACGAAATTAACTGGGGTGGCCGTCACAGCATCTGGCGTCACACCAGCCAGAACACCAACAAGCACGTTGAGGTGAACTTCATCGAAAAATTTACCACCGAGCGTTACTTCTGCCCGAACACCCGTTGCAGCATTACCTGGTTTCTGAGCTGGAGCCCGTGCGGTGAATGCAGCCGTGCGATCACCGAGTTCCTGAGCCGTTATCCGCACGTTACCCTGTTTATCTACATTGCGCGTCTGTATCACCACGCGGACCCGCGTAACCGTCAAGGTCTGCGTGATCTGATCAGCAGCGGCGTGACCATCCAGATTATGACCGAGCAAGAAAGCGGTTACTGCTGGCGTAACTTCGTTAACTATAGCCCGAGCAACGAAGCGCATTGGCCGCGTTACCCGCACCTGTGGGTGCGTCTGTACGTTCTGGAGCTGTATTGCATCATTCTGGGCCTGCCGCCGTGCCTGAACATTCTGCGTCGTAAGCAGCCGCAACTGACCTTCTTTACCATCGCGCTGCAGAGCTGCCACTACCAACGTCTGCCGCCGCACATTCTGTGGGCGACCGGTCTGAAGAGCGGCAGCGAAACCCCGGGTACCAGCGAAAGCGCGACCCCGGAGCCGCACCCGGTGCTGGAGAAACTGCGTAGCATCAACAACTATAACCCGAAGGACTTCGATTGGAACCTGAAACACGGTCGTGTTTTTATCATTAAGAGCTACAGCGAAGACGATATCCACCGTAGCATTAAATATAACATCTGGTGCAGCACCGAGCACGGCAACAAGCGTCTGGACGCGGCGTACCGTAGCATGAACGGTAAAGGCCCGGTGTATCTGCTGTTCAGCGTTAACGGTAGCGGCCACTTTTGCGGTGTGGCGGAAATGAAAAGCGCGGTTGATTACAACACCTGCGCGGGTGTGTGGAGCCAGGACAAGTGGAAAGGCCGTTTCGATGTTCGTTGGATTTTTGTGAAGGACGTTCCGAACAGCCAACTGCGTCACATCCGTCTGGAGAACAACGAAAACAAACCGGTGACCAACAGCCGTGATACCCAGGAAGTGCCGCTGGAAAAGGCGAAACAAGTTCTGAAGATCATTGCGAGCTACAAACACACCACCAGCATCTTCGACGATTTTAGCCACTATGAGAAGCGTCAGGAAGAGGAAGAGAGCGTGAAGAAGGAGCGTCAAGGTCGTGGCAAACTGGAGTACCCGTATGACGTTCCGGATTATGCGTAAATTGGAAGTGGATAA

SEQ ID NO: 26 - DNA, 1293 bp, synthetic construct
  source 1..1293: mol_type = other DNA
atgagcagcgaaaccggtccggtggcggttgacccgaccctgcgtcgtcgtattgagccgcacgagttcgaagtgttctttgatccgcgtgagctgcgtaaggaaacctgcctgctgtacgaaattaactggggtggccgtcacagcatctggcgtcacaccagccagaacaccaacaagcacgttgaggtgaacttcatcgaaaaatttaccaccgagcgttacttctgcccgaacacccgttgcagcattacctggtttctgagctggagcccgtgcggtgaatgcagccgtgcgatcaccgagttcctgagccgttatccgcacgttaccctgtttatctacattgcgcgtctgtatcaccacgcggacccgcgtaaccgtcaaggtctgcgtgatctgatcagcagcggcgtgaccatccagattatgaccgagcaagaaagcggttactgctggcgtaacttcgttaactatagcccgagcaacgaagcgcattggccgcgttacccgcacctgtgggtgcgtctgtacgttctggagctgtattgcatcattctgggcctgccgccgtgcctgaacattctgcgtcgtaagcagccgcaactgaccttctttaccatcgcgctgcagagctgccactaccaacgtctgccgccgcacattctgtgggcgaccggtctgaagagcggcagcgaaaccccgggtaccagcgaaagcgcgaccccggagggtcgtgtttttatcattaagagctacagcgaagacgatatccaccgtagcattaaatataacatctggtgcagcaccgagcacggcaacaagcgtctggacgcggcgtaccgtagcatgaacggtaaaggcccggtgtatctgctgttcagcgttaacggtagcggccacttttgcggtgtggcggaaatgaaaagcgcggttgattacaacacctgcgcgggtgtgtggagccaggacaagtggaaaggccgtttcgatgttcgttggatttttgtgaaggacgttccgaacagccaactgcgtcacatccgtctggagaacaacgaaaacaaaccggtgaccaacagccgtgatacccaggaagtgccgctggaaaaggcgaaacaagttctgaagatcattgcgagctacaaacacaccaccagcatcttcgacgattttagccactatgagaagcgtcaggaagaggaagagagcgtgaagaaggagcgtcaaggtcgtggcaaactggagtacccgtatgacgttccggattatgcgtaaattggaagtggataa

SEQ ID NO: 27 - protein, 7 aa, synthetic construct
PKKKRKV

SEQ ID NO: 28 - protein, 9 aa, synthetic construct
LPPLERLTL

SEQ ID NO: 29 - protein, 27 aa, synthetic construct
MDPVVVLGLCLSCLLLLSLWKQSYGGG

SEQ ID NO: 30 - protein, 21 aa, synthetic construct
METDTLLLWVLLLWVPGSTGD

SEQ ID NO: 31 - protein, 10 aa, synthetic construct
EQKLISEEDL

SEQ ID NO: 32 - protein, 14 aa, synthetic construct
GKPIPNPLLGLDST

SEQ ID NO: 33 - protein, 9 aa, synthetic construct
IPNPLLGLD

SEQ ID NO: 34 - protein, 8 aa, synthetic construct
DYKDDDDK

SEQ ID NO: 35 - protein, 22 aa, synthetic construct
DYKDHDGDYKDHDIDYKDDDDK

SEQ ID NO: 36 - protein, 158 aa, synthetic construct
ISLIAALAVDHVIGMETVMPWNLPADLAWFKRNTLNKPVIMGRHTWESIGRPLPGRKNIILSSQPSTDDRVTWVKSVDEAIAACGDVPEIMVIGGGRVYEQFLPKAQKLYLTHIDAEVEGDTHFPDYEPDDWESVFSEFHDADAQNSHSYCFEILERR

SEQ ID NO: 37 - RNA, 12 nt, synthetic construct
  source 1..12: mol_type = other RNA
gacttatgacag

SEQ ID NO: 38 - RNA, 12 nt, synthetic construct
  source 1..12: mol_type = other RNA
gacttacgacag

SEQ ID NO: 39 - DNA, 15 bp, synthetic construct
  source 1..15: mol_type = other DNA
ggacttacgacagtt

SEQ ID NO: 40 - RNA, 12 nt, synthetic construct
  source 1..12: mol_type = other RNA
gacttacgacag

SEQ ID NO: 41 - RNA, 12 nt, synthetic construct
  source 1..12: mol_type = other RNA
gacttatgacag

SEQ ID NO: 42 - RNA, 12 nt, synthetic construct
  source 1..12: mol_type = other RNA
ggcttacgacag

SEQ ID NO: 43 - RNA, 12 nt, synthetic construct
  source 1..12: mol_type = other RNA
gacttacgagag

SEQ ID NO: 44 - protein, 6 aa, synthetic construct
HHHHHH

SEQ ID NO: 45 - DNA, 12604 bp, synthetic construct
  source 1..12604: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcgctagcgtttaaacgggccctctagagccgccatggtgagcaagggcgaggagctgttcaccggggtggtgcccatcctggtcgagctggatggcgatgtaaatggccacaagttcagcgtgtccggcgagggcgagggcgatgccacctacggcaagctcaccctgaagttcatctgcaccaccggcaagctgcccgtgccctggcccaccctcgtcaccaccctcacctacggcgtgcagtgcttcagccgctaccccgatcacatgaagcagcacgatttcttcaagtccgccatgcccgaaggctacgtccaggagcgcaccatcttcttcaaggatgatggcaattaccgtacccgcgccgaggtgaagttcgagggcgataccctggtgaatcgcatcgagctgaagggcatcgatttcaaggaggatggcaatatcctggggcacaagctggagtacaattacaatagccacaatgtctatatcatggccgataagcagaagaatggcatcaaggtgaatttcaagatccgccacaatatcgaggatggcagcgtgcagctcgccgatcactaccagcagaatacccccatcggcgatggccccgtgctgctgcccgataatcactacctgagcacccagtccgccctgagcaaagatcccaatgagaagcgcgatcacatggtcctgctggagttcgtcaccgccgccgggatcactctcggcatggatgagctgtacaaggcggacttacgacagttgcgttacaccctttctcgacaaaacctaacttgcgcagaaaacatgccaatctcatcttggcttatcagtctgattgcggcgttagcggtagatcacgttatcggcatggaaaccgtcatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgagtacggacgatcgcgtaacgtgggtgaagtcggtggatgaagccatcgcggcgtgtggtgacgtaccagaaatcatggttattggcggcggtcgcgtttatgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcgataagcctcattgtgcattctctcgagtacccctacgacgtgcccgactacgcctgagggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaccagtgtgatggatatctgcagaattcgcccttggatccgaattcctgcagccccgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgagggggatccactagcatgaagggcgaattccagcacactggtaacccgtgtcggctccagatctggcctccgcgccgggttttggcgcctcccgcgggcgcccccctcctcacggcgagccgcgttgacattgattattgactaggcttttgcaaaaagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctaggtaagcttggtaccgagctcggatccactagtccagtgtggtggaattctgcagatatccagcacagtggggtttagtgaaccgtcagatccgctagagatccgcggccgctaatacgactcactatagggagagccgccaccatgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaaagcggcagcgagactcccgggacctcagagtccgccacaccagaaccccacccagtgttggagaagcttcggtccattaataactataaccccaaagattttgactggaatctgaaacatggccgggttttcatcattaagagctactctgaggacgatattcaccgttccattaagtataatatttggtgcagcacagagcatggtaacaagagactggatgctgcttatcgttccatgaacgggaaaggccccgtttacttacttttcagtgtcaacggcagtggacacttctgtggcgtggcagaaatgaaatctgctgtggactacaacacatgtgcaggtgtgtggtcccaggacaaatggaagggtcgttttgatgtcaggtggatttttgtgaaggacgttcccaatagccaactgcgacacattcgcctagagaacaacgagaataaaccagtgaccaactctagggacactcaggaagtgcctctggaaaaggctaagcaggtgttgaaaattatagccagctacaagcacaccacttccatttttgatgacttctcacactatgagaaacgccaagaggaagaagaaagtgttaaaaaggaacgtcaaggtcgtgggaaactcgagtacccctacgacgtgcccgactacgcctgagtttaaaatcgatggtacactcgaggttaacgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcatcccgccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgccggttccggcgcaacaaacttctctctgctgaaacaagccggagatgtcgaagagaatcctggaccgatggctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcgccaaaaaagaagagaaaggtcgacggcggtggtgctttgtctcctcagcactctgctgtcactcaaggaagtatcatcaagaacaaggagggcatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccctggctggtgtaaagtagatgccgaccgaacaagagctgatttcgagaacgcctcagccagcaactcgcgcgagcctagcaaggcaaatgcgagagaacggccttacgcttggtggcacagttctcgtccacagttcgctaagctcgctcggctgggtcgcgggagggccggtcgcagtgattcaggcccttctggattgtgttggtccccagggcacgattgtcatgcccacgcactcgggtgatctgactgatcccgcagattggagatcgccgcccgtgcctgccgattgggtgcagatccgtcgagttaacaaaagaaaaggggggactggaagggctaattcactcccaacgaagacaagatatcataacttcgtatagcatacattatacgaagttatcggctagctggtccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 46 - DNA, 9483 bp, synthetic construct
  source 1..9483: mol_type = other DNA
agggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgagggggatccactagcatgaagggcgaattccagcacactggtaacccgtgtcggctccagatctggcctccgcgccgggttttggcgcctcccgcgggcgcccccctcctcacggcgagccgcgttgacattgattattgactaggcttttgcaaaaagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctaggtaagcttggtaccgagctcggatccactagtccagtgtggtggaattctgcagatatccagcacagtggggtttagtgaaccgtcagatccgctagagatccgcggccgctaatacgactcactatagggagagccgccaccatgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaaagcggcagcgagactcccgggacctcagagtccgccacaccagaaccccacccagtgttggagaagcttcggtccattaataactataaccccaaagattttgactggaatctgaaacatggccgggttttcatcattaagagctactctgaggacgatattcaccgttccattaagtataatatttggtgcagcacagagcatggtaacaagagactggatgctgcttatcgttccatgaacgggaaaggccccgtttacttacttttcagtgtcaacggcagtggacacttctgtggcgtggcagaaatgaaatctgctgtggactacaacacatgtgcaggtgtgtggtcccaggacaaatggaagggtcgttttgatgtcaggtggatttttgtgaaggacgttcccaatagccaactgcgacacattcgcctagagaacaacgagaataaaccagtgaccaactctagggacactcaggaagtgcctctggaaaaggctaagcaggtgttgaaaattatagccagctacaagcacaccacttccatttttgatgacttctcacactatgagaaacgccaagaggaagaagaaagtgttaaaaaggaacgtcaaggtcgtgggaaactcgagtacccctacgacgtgcccgactacgcctgagtttaaaatcgatggtacactcgaggttaacgaattctaccttacccagagtgcaggtgtgtggagatccctcctgccttgacattgagcagccttagagggtgggggaggctcaggggtcaggtctctgttcctgcttattggggagttcctggcctggcccttctatgtctccccaggtaccccagtttttctgggttcacccagagtgcagatgcttgaggaggtgggaagggactatttgggggtgtctggctcaggtgccatgcctcactggggctggttggcacctgcatttcctgggagtggggctgtctcagggtagctgggcacggtgttcccttgagtgggggtgtagtgagtgttcctagctgccacgcctttgccttcacctatgggatcgtggctgtcagttaattaaccttccgcgggagctcacggggagagccccccgccaaagcccccagggatgtaattgcatccctcttccgctagggggcagcagcgagccgcccggggctccgctccggtccggcgctccccccgcatccccgagccggagccggcagcgtgcggggacagcccggcacggggaaggtggcacgcgatcgctttcctctgaacgcttctcgctgctctttgagcctgcagacacctggggggatacggggaaaaagctttaggctgaaagagagatttagaatgacagaatcatagaatggcctgggttgcaaaggagcacagtgctcacccagctccaaccccctgctatgtgcagggtcgccaaccagcagcccaggctgcccagagccacatccagcctggccttgaatgcctgcagggatggggcatccacagcctccttgggcaacctgttcagtgcgtcacggatccaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagctgtagctagcacaaccatggatagcactgagaacgtcatcaagcccttcatgcgcttcaaggtgcacatggagggctccgtgaacggccacgagttcgagatcgagggcgagggcgagggcaagccctacgagggcacccagaccgccaagctgcaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggcaccttcatctaccacgtgaagttcatcggcgtgaacttcccctccgacggccccgtaatgcagaagaagactctgggctgggagccctccaccgagcgcctgtacccccgcgacggcgtgctgaagggcgagatccacaaggcgctgaagctgaagggcggcggccactacctggtggagttcaagtcaatctacatggccaagaagcccgtgaagctgcccggctactactacgtggactccaagctggacatcacctcccacaacgaggactacaccgtggtggagcagtacgagcgcgccgaggcccgccaccacctgttccagtagggctagctggtccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 47 - DNA, 9483 bp, synthetic construct; identical to SEQ ID NO: 46
  source 1..9483: mol_type = other DNA

SEQ ID NO: 48 - DNA, 852 bp, synthetic construct
  source 1..852: mol_type = other DNA
atggtgagcaagggcgaggagctgttcaccggggtggtgcccatcctggtcgagctggatggcgatgtaaatggccacaagttcagcgtgtccggcgagggcgagggcgatgccacctacggcaagctcaccctgaagttcatctgcaccaccggcaagctgcccgtgccctggcccaccctcgtcaccaccctcacctacggcgtgcagtgcttcagccgctaccccgatcacatgaagcagcacgatttcttcaagtccgccatgcccgaaggctacgtccaggagcgcaccatcttcttcaaggatgatggcaattaccgtacccgcgccgaggtgaagttcgagggcgataccctggtgaatcgcatcgagctgaagggcatcgatttcaaggaggatggcaatatcctggggcacaagctggagtacaattacaatagccacaatgtctatatcatggccgataagcagaagaatggcatcaaggtgaatttcaagatccgccacaatatcgaggatggcagcgtgcagctcgccgatcactaccagcagaatacccccatcggcgatggccccgtgctgctgcccgataatcactacctgagcacccagtccgccctgagcaaagatcccaatgagaagcgcgatcacatggtcctgctggagttcgtcaccgccgccgggatcactctcggcatggatgagctgtacaagttgcttagccatggcttcccgccggaggtggaggagcaggatgatggcacgctgcccatgtcttgtgcccaggagagcgggatggaccgtcaccctgcagcctgtgcttctgctaggatcaatgtgttagatgcg

SEQ ID NO: 49 - protein, 284 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYRTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKLLSHGFPPEVEEQDDGTLPMSCAQESGMDRHPAACASARINVLDA

SEQ ID NO: 50 - DNA, 81 bp, synthetic construct
  source 1..81: mol_type = other DNA
gcggacttacgacagttgcgttacaccctttctcgacaaaacctaacttgcgcagaaaacatgccaatctcatcttggctt

SEQ ID NO: 51 - DNA, 81 bp, synthetic construct
  source 1..81: mol_type = other DNA
gcggcgttacgacagttgcgttacaccctttctcgacaaaacctaacttgcgcagaaaacatgccaatctcatcttggctt

SEQ ID NO: 52 - DNA, 81 bp, synthetic construct
  source 1..81: mol_type = other DNA
gcggacttacgtcagttgcgttacaccctttctcgacaaaacctaacttgcgcagaaaacatgccaatctcatcttggctt

SEQ ID NO: 53 - DNA, 81 bp, synthetic construct
  source 1..81: mol_type = other DNA
gcggagttacgacagttgcgttacaccctttctcgtcaaaacctaacttgcgcagaaaacatgccaatctcatcttggctt

SEQ ID NO: 54 - DNA, 81 bp, synthetic construct
  source 1..81: mol_type = other DNA
gcggagttacgacagttgcgttacaccctttctcgacaaagcctaacttgcgcagaaaacatgccaatctcatcttggctt

SEQ ID NO: 55 - DNA, 81 bp, synthetic construct
  source 1..81: mol_type = other DNA
gcggagttacgacagttgcgttacaccctttctcgacaaaacctagcttgcgcagaaaacatgccaatctcatcttggctt

SEQ ID NO: 56 - DNA, 81 bp, synthetic construct
  source 1..81: mol_type = other DNA
gcggagttacgacagttgcgttacaccctttctcgacaaaacctaacttgcgcagaaagcatgccaatctcatcttggctt

SEQ ID NO: 57 - DNA, 39 bp, synthetic construct
  source 1..39: mol_type = other DNA
gcggacttacgacagttgcgtccaatctcatcttggctt

SEQ ID NO: 58 - DNA, 81 bp, synthetic construct
  source 1..81: mol_type = other DNA
gcggccttacgtcagttgcgttacaccctttctcggcaaagcctagcttgcgcagaaagcatgccaatctcatcttggctt

SEQ ID NO: 59 - protein, 100 aa, synthetic construct; identical to SEQ ID NO: 21

SEQ ID NO: 60 - protein, 215 aa, synthetic construct
RRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 61 - protein, 179 aa, synthetic construct
LMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPL

SEQ ID NO: 62 - protein, 173 aa, synthetic construct
TSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLR

SEQ ID NO: 63 - protein, 181 aa, synthetic construct; identical to SEQ ID NO: 17